(19) United States
(12) Patent Application Publication
(10) Pub. No.: US 2022/0153754 A1
(43) Pub. Date: May 19, 2022

(54) FUSED HETEROCYCLE DERIVATIVES AS CAPSID ASSEMBLY MODULATORS

(71) Applicant: JANSSEN SCIENCES IRELAND UNLIMITED COMPANY, Co Cork (IE)

(72) Inventor: Scott D. KUDUK, Harleysville, PA (US)

(21) Appl. No.: 17/595,819

(22) PCT Filed: May 27, 2020
(86) PCT No.: PCT/EP2020/064751

§ 371 Date: Nov. 24, 2021

Related U.S. Application Data
(60) Provisional application No. 62/853,528, filed on May 28, 2019.

(51) Int. Cl. C07D 498/22; C07D 498/14; C07D 471/22

(52) U.S. Cl. CPC C07D 498/22; A61K 45/06; C07D 471/22; C07D 498/14

(57) ABSTRACT

Disclosed are compounds, compositions and methods for treating of diseases, syndromes, conditions, and disorders that are affected by the modulation of CAM1. Such compounds are represented by Formula (I) as follows:

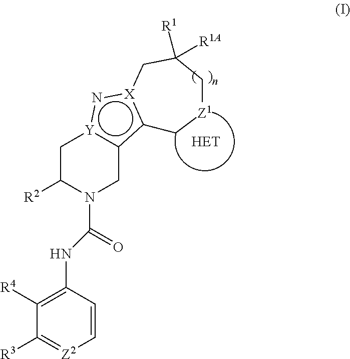

wherein, R¹, R^{1A}, R², R³, R⁴, HET, n, X, Y, Z¹ and Z² are defined herein.

### RELATED APPLICATIONS

This application claims priority to U.S. Provisional Application No. 62/853,528 filed on May 28, 2019, the content of which is hereby incorporated in its entirety.

### FIELD OF THE PRESENT DISCLOSURE

The present disclosure is related to azepine compounds, pharmaceutical compositions comprising these compounds, chemical processes for preparing these compounds and their use in the treatment of diseases associated with HBV infection in animals, in particular humans.

### BACKGROUND

Chronic hepatitis B virus (HBV) infection is a significant global health problem, affecting over 5% of the world population (over 350 million people worldwide and 1.25 million individuals in the U.S.).

Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in part to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress. Capsid structures also respond to environmental cues to allow un-coating after viral entry. Consistently, the appropriate timing of capsid assembly and dis-assembly, the appropriate capsid stability and the function of core protein have been found to be critical for viral infectivity.

The crucial function of HBV capsid proteins imposes stringent evolutionary constraints on the viral capsid protein sequence, leading to the observed low sequence variability and high conservation. Consistently, mutations in HBV capsid that disrupt its assembly are lethal, and mutations that perturb capsid stability severely attenuate viral replication. The high functional constraints on the multi-functional HBV core/capsid protein is consistent with a high sequence conservation, as many mutations are deleterious to function. Indeed, the core/capsid protein sequences are >90% identical across HBV genotypes and show only a small number of polymorphic residues. Resistance selection to HBV core/capsid protein binding compounds may therefore be difficult to select without large impacts on virus replication fitness.

Reports describing compounds that bind viral capsids and inhibit replication of HIV, rhinovirus and HBV provide strong pharmacological proof of concept for viral capsid proteins as antiviral drug targets.

There is a need in the art for therapeutic agents that can increase the suppression of virus production and that can treat, ameliorate, and/or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

In view of the clinical importance of HBV, the identification of compounds that can increase the suppression of virus production and that can treat, ameliorate, and/or prevent HBV infection represents an attractive avenue into the development of new therapeutic agents. Such compounds are provided herein.

### SUMMARY

The present disclosure is directed to the general and preferred embodiments defined, respectively, by the independent and dependent claims appended hereto, which are incorporated by reference herein. In particular, the present disclosure is directed to compounds of Formula (I):

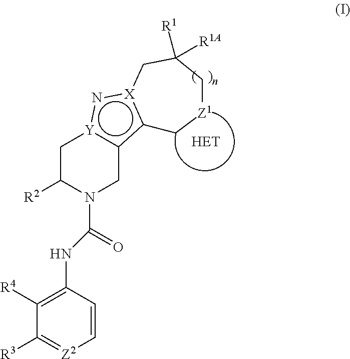

and pharmaceutically acceptable salts, stereoisomers, isotopic variants, N-oxides, or solvates of compounds of Formula (I);
wherein
R¹ is independently selected from the group consisting of: hydrogen, C₁₋₄alkyl, hydroxy, hydroxymethyl, (2,2-difluoroethoxy)methyl, OC₁₋₄alkyl, and fluoro;
R^{1A} is independently hydrogen or taken together with R¹ to form methylenyl;
n is an integer that is 0, 1, or 2;
R² is independently selected from the group consisting of: hydrogen and C₁₋₆alkyl;
R³ is selected from the group consisting of: Cl, CN, and C₁₋₄haloalkyl;
R⁴ is H, or F;
HET is a 5- or 6-membered heteroaryl, optionally independently substituted with one to two substituents selected from the group consisting of: C₁₋₄alkyl, bromo, chloro, fluoro, and hydroxy(C₁₋₄)alkyl;
X and Y are each independently selected from: N or C, such that only one of X and Y is N in any instance;
Z¹ is N or C; and
Z² is N or CF.

Further embodiments include pharmaceutically acceptable salts of compounds of Formula (I), pharmaceutically acceptable prodrugs of compounds of Formula (I), pharmaceutically active metabolites of compounds of Formula (I), and enantiomers and diastereomers of the compounds of Formula (I), as well as pharmaceutically acceptable salts thereof.

In embodiments, the compounds of Formula (I) are compounds selected from those species described or exemplified in the detailed description below.

The present disclosure is also directed to pharmaceutical compositions comprising one or more compounds of Formula (I), pharmaceutically acceptable salts of compounds of Formula (I), pharmaceutically acceptable prodrugs of compounds of Formula (I), and pharmaceutically active metabolites of Formula (I). Pharmaceutical compositions may further comprise one or more pharmaceutically acceptable excipients or one or more other agents or therapeutics.

The present disclosure is also directed to methods of using or uses of compounds of Formula (I). In embodiments, compounds of Formula (I) are used to treat or ameliorate hepatitis B viral (HBV) infection, increase the suppression of HBV production, interfere with HBV capsid assembly or other HBV viral replication steps or products thereof. The methods comprise administering to a subject in need of such method an effective amount of at least one compound of Formula (I), pharmaceutically acceptable salts of compounds of Formula (I), pharmaceutically acceptable prodrugs of compounds of Formula (I), and pharmaceutically active metabolites of compounds of Formula (I). Additional embodiments of methods of treatment are set forth in the detailed description.

An object of the present disclosure is to overcome or ameliorate at least one of the disadvantages of the conventional methodologies and/or prior art, or to provide a useful alternative thereto. Additional embodiments, features, and advantages of the present disclosure will be apparent from the following detailed description and through practice of the disclosed subject matter.

### DETAILED DESCRIPTION

Additional embodiments, features, and advantages of the subject matter of the present disclosure will be apparent from the following detailed description of such disclosure and through its practice. For the sake of brevity, the publications, including patents, cited in this specification are herein incorporated by reference.

Provided herein are compounds of Formula (I), and their pharmaceutically acceptable salts, pharmaceutically acceptable prodrugs, and pharmaceutically active metabolites of the disclosed compounds.

In one aspect, provided herein are compounds of Formula (I), and pharmaceutically acceptable salts, stereoisomers, isotopic variants, N-oxides, or solvates thereof,

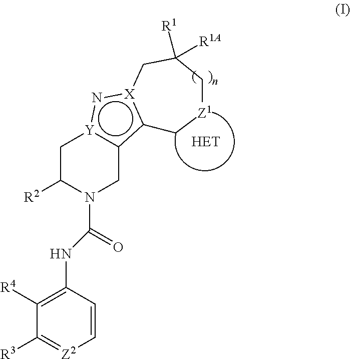

and pharmaceutically acceptable salts, stereoisomers, isotopic variants, N-oxides, or solvates of compounds of Formula (I);
wherein
R¹ is independently selected from the group consisting of: hydrogen, C₁₋₄alkyl, hydroxy, hydroxymethyl, (2,2-difluoroethoxy)methyl, OC₁₋₄alkyl, and fluoro;
R^{1A} is independently hydrogen or taken together with R¹ to form methylenyl;
n is an integer that is 0, 1, or 2;
R² is independently selected from the group consisting of: hydrogen and C₁₋₆alkyl;
R³ is selected from the group consisting of Cl, CN, and C₁₋₄haloalkyl;
R⁴ is H, or F;
HET is a 5- or 6-membered heteroaryl, optionally independently substituted with one to two substituents selected from C₁₋₄alkyl, bromo, chloro, fluoro, and hydroxy(C₁₋₄)alkyl;
X and Y are each independently selected from: N or C, such that only one of X and Y is N in any instance;
Z¹ is N or C; and
Z² is N or CF.

In embodiments, the compound of Formula (I) is a compound wherein R¹ is hydrogen, C₁₋₄alkyl, hydroxy, hydroxymethyl, (2,2-difluoroethoxy)methyl, OC₁₋₄alkyl, or fluoro.

In embodiments, the compound of Formula (I) is a compound wherein R¹ and R^{1A} are taken together with R¹ to form methylenyl.

In embodiments, the compound of Formula (I) is a compound wherein n is 1.

In embodiments, the compound of Formula (I) is a compound wherein n is 0.

In embodiments, the compound of Formula (I) is a compound wherein n is 2.

In embodiments, the compound of Formula (I) is a compound wherein R² is H or CH₃.

In embodiments, the compound of Formula (I) is a compound wherein R² is H.

In embodiments, the compound of Formula (I) is a compound wherein R² is CH₃.

In embodiments, the compound of Formula (I) is a compound wherein R³ is Cl, CN, or CF₃.

In embodiments, the compound of Formula (I) is a compound wherein R⁴ is H.

In embodiments, the compound of Formula (I) is a compound wherein R⁴ is F.

In embodiments, the compound of Formula (I) is a compound wherein Y is N and X is C.

In embodiments, the compound of Formula (I) is a compound wherein Y is C and X is N.

In embodiments, the compound of Formula (I) is a compound wherein Z¹ is N.

In embodiments, the compound of Formula (I) is a compound wherein Z¹ is C.

In embodiments, the compound of Formula (I) is a compound wherein Z² is N.

In embodiments, the compound of Formula (I) is a compound wherein Z² is CF.

In embodiments, the compound of Formula (I) is a compound wherein

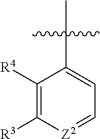

is 3-cyano-4-fluorophenyl, 4-fluoro-3-(trifluoromethyl)phenyl, or 3-chloro-4-fluorophenyl.

In embodiments, the compound of Formula (I) is a compound wherein

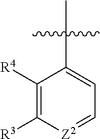

is 3-cyano-4-fluorophenyl.

In embodiments, the compound of Formula (I) is a compound wherein HET is a heteroaryl independently selected from the group consisting of isoxazolyl, pyridinyl, triazolyl, 3-methyl-triazolyl, pyridazinyl, pyrazolyl, or 1-methylpyrazolyl.

In embodiments, the compound of Formula (I) is a compound wherein HET is a heteroaryl independently selected from the group consisting of isoxazolyl and pyrazolyl.

A further embodiment of the present disclosure is a compound selected from the group consisting of:

TABLE 1

| Ex # | Structure | Name |
|---|---|---|
| 1 | 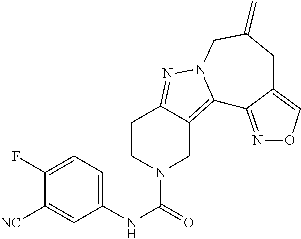 [SMILES: C=C1Cc2conc2-c2c3c(nn2C1)CCN(C(=O)Nc1ccc(F)c(C#N)c1)C3] | N-(3-Cyano-4-fluorophenyl)-5- methylene-5,6,9,10-tetrahydro- 4H-isoxazolo[3,4- c]pyrido[4′,3′:3,4]pyrazolo[1,5- a]azepine-11(12H)-carboxamide; |
| 2 | 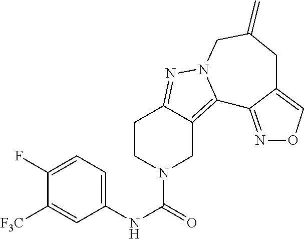 [SMILES: C=C1Cc2conc2-c2c3c(nn2C1)CCN(C(=O)Nc1ccc(F)c(C(F)(F)F)c1)C3] | N-(4-Fluoro-3- (trifluoromethyl)phenyl)-5- methylene-5,6,9,10-tetrahydro- 4H-isoxazolo[3,4- c]pyrido[4′,3′:3,4]pyrazolo[1,5- a]azepine-11(12H)-carboxamide; |
| 3 | 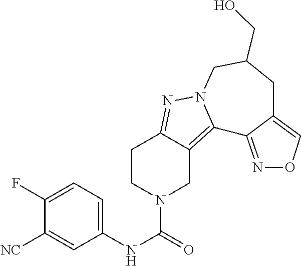 [SMILES: N#Cc1cc(NC(=O)N2CCc3nn4c(c3C2)-c2nocc2CC(CO)C4)ccc1F] | N-(3-Cyano-4-fluorophenyl)-5- (hydroxymethyl)-5,6,9,10- tetrahydro-4H-isoxazolo[3,4- c]pyrido[4′,3′:3,4]pyrazolo[1,5- a]azepine-11(12H)-carboxamide; |
| 4 | 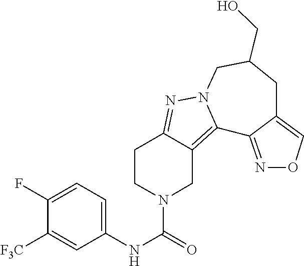 [SMILES: O=C(Nc1ccc(F)c(C(F)(F)F)c1)N1CCc2nn3c(c2C1)-c1nocc1CC(CO)C3] | N-(4-Fluoro-3- (trifluoromethyl)phenyl)-5- (hydroxymethyl)-5,6,9,10- tetrahydro-4H-isoxazolo[3,4- c]pyrido[4′,3′:3,4]pyrazolo[1,5- a]azepine-11(12H)-carboxamide; |
| 5 | 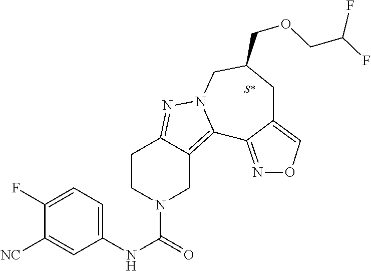 [SMILES: N#Cc1cc(NC(=O)N2CCc3nn4c(c3C2)-c2nocc2C[C@H](COCC(F)F)C4)ccc1F] | (5S*)-N-(3-Cyano-4- fluorophenyl)-5-((2,2- difluoroethoxy)methyl)-5,6,9,10- tetrahydro-4H-isoxazolo[3,4- c]pyrido[4′,3′:3,4]pyrazolo[1,5- a]azepine-11(12H)-carboxamide; |
| 6 | 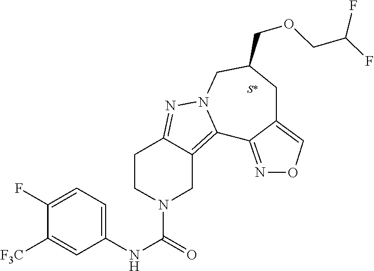 [SMILES: O=C(Nc1ccc(F)c(C(F)(F)F)c1)N1CCc2nn3c(c2C1)-c1nocc1C[C@H](COCC(F)F)C3] | (5S*)-5-((2,2- Difluoroethoxy)methyl)-N-(4- fluoro-3- (trifluoromethyl)phenyl)- 5,6,9,10-tetrahydro-4H- isoxazolo[3,4- c]pyrido[4′,3′:3,4]pyrazolo[1,5- a]azepine-11(12H)-carboxamide; |
| 7 | 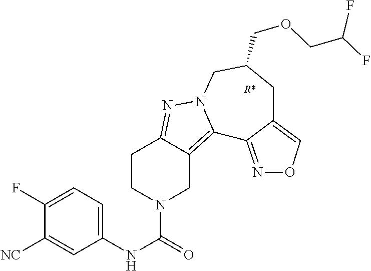 [SMILES: N#Cc1cc(NC(=O)N2CCc3nn4c(c3C2)-c2nocc2C[C@@H](COCC(F)F)C4)ccc1F] | (5R*)-N-(3-Cyano-4- fluorophenyl)-5-((2,2- difluoroethoxy)methyl)-5,6,9,10- tetrahydro-4H-isoxazolo[3,4- c]pyrido[4′,3′:3,4]pyrazolo[1,5- a]azepine-11(12H)-carboxamide; |
| 8 | 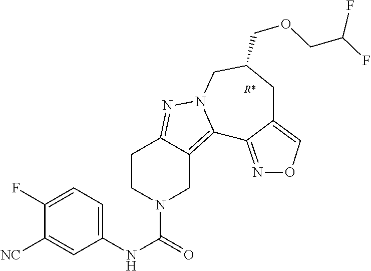 [SMILES: N#Cc1cc(NC(=O)N2CCc3nn4c(c3C2)-c2nocc2C[C@@H](COCC(F)F)C4)ccc1F] | (5R*)-5-((2,2- Difluoroethoxy)methyl)-N-(4- fluoro-3- (trifluoromethyl)phenyl)- 5,6,9,10-tetrahydro-4H- isoxazolo[3,4- c]pyrido[4′,3′:3,4]pyrazolo[1,5- a]azepine-11(12H)-carboxamide; |
| 9 | 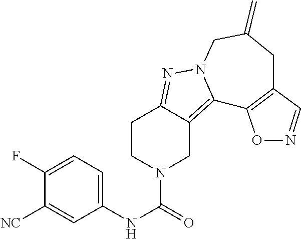 [SMILES: C=C1Cc2cnoc2-c2c3c(nn2C1)CCN(C(=O)Nc1ccc(F)c(C#N)c1)C3] | N-(3-Cyano-4-fluorophenyl)-5- methylene-5,6,9,10-tetrahydro- 4H-isoxazolo[5,4- c]pyrido[4′,3′:3,4]pyrazolo[1,5- a]azepine-11(12H)-carboxamide; |
| 10 | 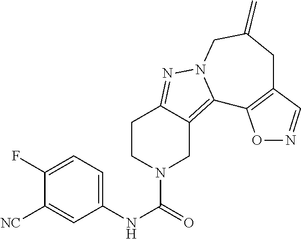 [SMILES: C=C1Cc2cnoc2-c2c3c(nn2C1)CCN(C(=O)Nc1ccc(F)c(C#N)c1)C3] | N-(4-Fluoro-3- (trifluoromethyl)phenyl)-5- methylene-5,6,9,10-tetrahydro- 4H-isoxazolo[5,4- c]pyrido[4′,3′:3,4]pyrazolo[1,5- a]azepine-11(12H)-carboxamide; |
| 11 | 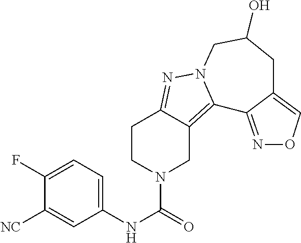 [SMILES: N#Cc1cc(NC(=O)N2CCc3nn4c(c3C2)-c2nocc2CC(O)C4)ccc1F] | N-(3-Cyano-4-fluorophenyl)-5- hydroxy-5,6,9,10-tetrahydro-4H- isoxazolo[3,4- c]pyrido[4′,3′:3,4]pyrazolo[1,5- a]azepine-11(12H)-carboxamide; |
| 12 | 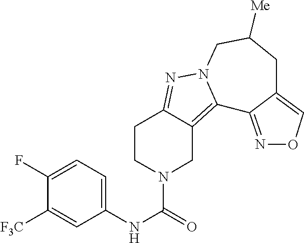 [SMILES: CC1Cc2conc2-c2c3c(nn2C1)CCN(C(=O)Nc1ccc(F)c(C(F)(F)F)c1)C3] | N-(4-Fluoro-3- (trifluoromethyl)phenyl)-5- methyl-5,6,9,10-tetrahydro-4H- isoxazolo[3,4- c]pyrido[4′,3′:3,4]pyrazolo[1,5- a]azepine-11(12H)-carboxamide; |
| 13 | 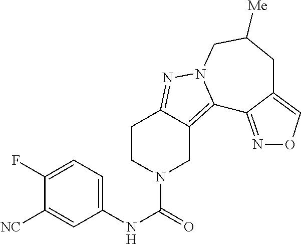 [SMILES: CC1Cc2conc2-c2c3c(nn2C1)CCN(C(=O)Nc1ccc(F)c(C#N)c1)C3] | N-(3-Cyano-4-fluorophenyl)-5- methyl-5,6,9,10-tetrahydro-4H- isoxazolo[3,4- c]pyrido[4′,3′:3,4]pyrazolo[1,5- a]azepine-11(12H)-carboxamide; |
| 14 | 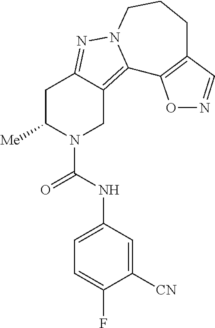 [SMILES: C[C@@H]1Cc2nn3c(c2CN1C(=O)Nc1ccc(F)c(C#N)c1)-c1oncc1CCC3] | (10R)-N-(3-Cyano-4- fluorophenyl)-10-methyl- 5,6,9,10-tetrahydro-4H- isoxazolo[5,4- c]pyrido[4′,3′:3,4]pyrazolo[1,5- a]azepine-11(12H)-carboxamide; |
| 15 | 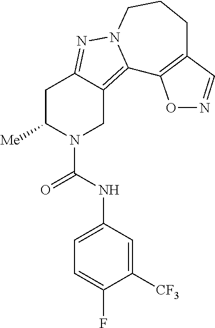 [SMILES: C[C@@H]1Cc2nn3c(c2CN1C(=O)Nc1ccc(F)c(C(F)(F)F)c1)-c1oncc1CCC3] | (10R)-N-(4-Fluoro-3- (trifluoromethyl)phenyl)-10- methyl-5,6,9,10-tetrahydro-4H- isoxazolo[5,4- c]pyrido[4′,3′:3,4]pyrazolo[1,5- a]azepine-11(12H)-carboxamide; |
| 16 | 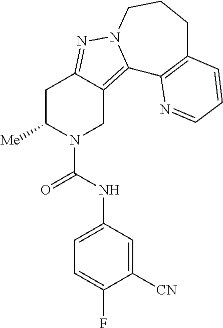 [SMILES: C[C@@H]1Cc2nn3c(c2CN1C(=O)Nc1ccc(F)c(C#N)c1)-c1ncccc1CCC3] | (11R)-N-(3-Cyano-4- fluorophenyl)-11-methyl- 6,7,10,11-tetrahydro-5H- pyrido[2,3- c]pyrido[4′,3′:3,4]pyrazolo[1,5- a]azepine-12(13H)-carboxamide; |
| 17 | 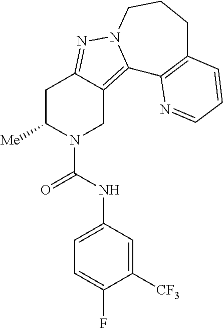 [SMILES: C[C@@H]1Cc2nn3c(c2CN1C(=O)Nc1ccc(F)c(C(F)(F)F)c1)-c1ncccc1CCC3] | (11R)-N-(4-Fluoro-3- (trifluoromethyl)phenyl)-11- methyl-6,7,10,11-tetrahydro-5H- pyrido[2,3- c]pyrido[4′,3′:3,4]pyrazolo[1,5- a]azepine-12(13H)-carboxamide; |
| 18 | 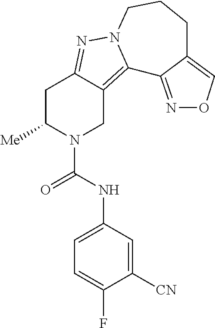 [SMILES: C[C@@H]1Cc2nn3c(c2CN1C(=O)Nc1ccc(F)c(C#N)c1)-c1nocc1CCC3] | (10R)-N-(3-Cyano-4- fluorophenyl)-10-methyl- 5,6,9,10-tetrahydro-4H- isoxazolo[3,4- c]pyrido[4′,3′:3,4]pyrazolo[1,5- a]azepine-11(12H)-carboxamide; |
| 19 | 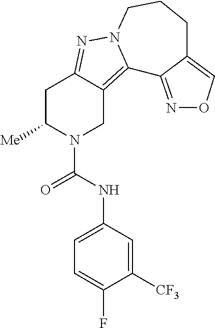 [SMILES: C[C@@H]1Cc2nn3c(c2CN1C(=O)Nc1ccc(F)c(C(F)(F)F)c1)-c1nocc1CCC3] | (10R)-N-(4- Fluoro-3- (trifluoromethyl)phenyl)-10- methyl-5,6,9,10-tetrahydro-4H- isoxazolo[3,4- c]pyrido[4′,3′:3,4]pyrazolo[1,5- a]azepine-11(12H)-carboxamide; |
| 20 | 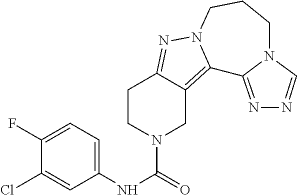 [SMILES: O=C(Nc1ccc(F)c(Cl)c1)N1CCc2nn3c(c2C1)-c1nncn1CCC3] | N-(3-Chloro-4-fluorophenyl)- 6,7,10,11-tetrahydro-5H- pyrido[4′,3′:3,4]pyrazolo[1,5- a][1,2,4]triazolo[3,4- c][1,4]diazepine-12(13H)- carboxamide; |
| 21 | 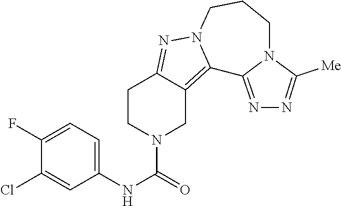 [SMILES: Cc1nnc2n1CCCn1nc3c(c1-2)CN(C(=O)Nc1ccc(F)c(Cl)c1)CC3] | N-(3-Chloro-4-fluorophenyl)-3- methyl-6,7,10,11-tetrahydro-5H- pyrido[4′,3′:3,4]pyrazolo[1,5- a][1,2,4]triazolo[3,4- c][1,4]diazepine-12(13H)- carboxamide; |
| 22 | 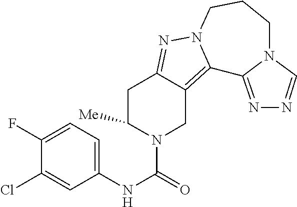 [SMILES: C[C@@H]1Cc2nn3c(c2CN1C(=O)Nc1ccc(F)c(Cl)c1)-c1nncn1CCC3] | (11R)-N-(3-Chloro-4- fluorophenyl)-11-methyl- 6,7,10,11-tetrahydro-5H- pyrido[4′,3′:3,4]pyrazolo[1,5- a][1,2,4]triazolo[3,4-c] [1,4]diazepine-12(13H)- carboxamide; |
| 23 | 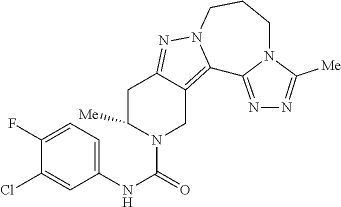 [SMILES: Cc1nnc2n1CCCn1nc3c(c1-2)CN(C(=O)Nc1ccc(F)c(Cl)c1)[C@H](C)C3] | (11R)-N-(3-Chloro-4- fluorophenyl)-11-methyl- 6,7,10,11-tetrahydro-5H- pyrido[4′,3′:3,4]pyrazolo[1,5- a][1,2,4]triazolo[3,4- c][1,4]diazepine-12(13H)- carboxamide; |
| 24 | 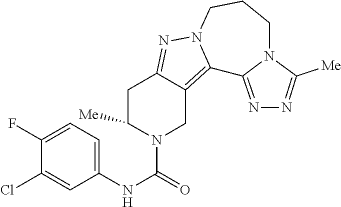 [SMILES: Cc1nnc2n1CCCn1nc3c(c1-2)CN(C(=O)Nc1ccc(F)c(Cl)c1)[C@H](C)C3] | N-(3-Cyano-4-fluorophenyl)- 6,7,10,11-tetrahydro-5H- pyridazino[3,4- c]pyrido[4′,3′:3,4]pyrazolo[1,5- a]azepine-12(13H)-carboxamide; |
| 25 | 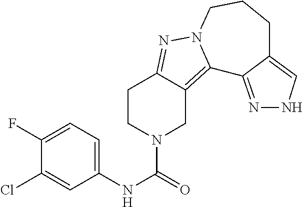 [SMILES: O=C(Nc1ccc(F)c(Cl)c1)N1CCc2nn3c(c2C1)-c1n[nH]cc1CCC3] | N-(3-Chloro-4-fluorophenyl)- 4,5,6,9,10,12- hexahydropyrazolo[3,4- c]pyrido[4′,3′:3,4]pyrazolo[1,5- a]azepine-11(2H)-carboxamide; |
| 26 | 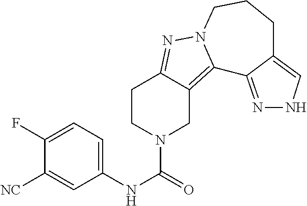 [SMILES: N#Cc1cc(NC(=O)N2CCc3nn4c(c3C2)-c2n[nH]cc2CCC4)ccc1F] | N-(3-Cyano-4-fluorophenyl)- 4,5,6,9,10,12- hexahydropyrazolo[3,4- c]pyrido[4′,3′:3,4]pyrazolo[1,5- a]azepine-11(2H)-carboxamide; |
| 27 | 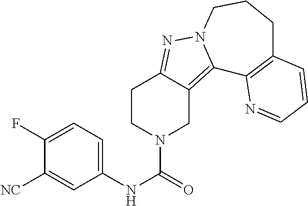 [SMILES: N#Cc1cc(NC(=O)N2CCc3nn4c(c3C2)-c2ncccc2CCC4)ccc1F] | N-(3-Cyano-4-fluorophenyl)- 6,7,10,11-tetrahydro-5H- pyrido[2,3- c]pyrido[4′,3′:3,4]pyrazolo[1,5- a]azepine-12(13H)-carboxamide; |
| 28 | 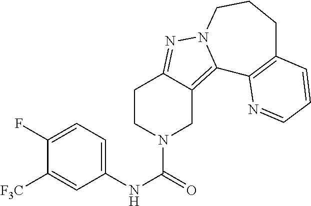 [SMILES: O=C(Nc1ccc(F)c(C(F)(F)F)c1)N1CCc2nn3c(c2C1)-c1ncccc1CCC3] | N-(4Fluoro-3- (trifluoromethyl)phenyl)- 6,7,10,11-tetrahydro-5H- pyrido[2,3-c]pyrido[4′,3′:3,4] pyrazolo[1,5-a]azepine-12(13H)- carboxamide; |
| 29 | 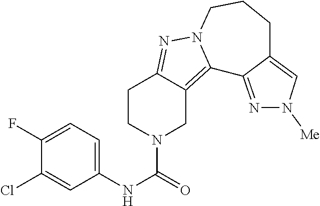 [SMILES: Cn1cc2c(n1)-c1c3c(nn1CCC2)CCN(C(=O)Nc1ccc(F)c(Cl)c1)C3] | N-(3-Chloro-4-fluorophenyl)-2- methyl-4,5,6,9,10,12- hexahydropyrazolo[3,4- c]pyrido[4′,3′:3,4]pyrazolo[1,5- a]azepine-11(2H)-carboxamide; |
| 30 | 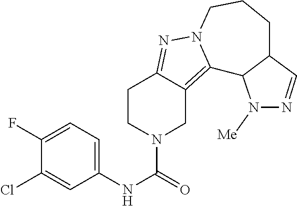 [SMILES: CN1N=CC2CCCn3nc4c(c3C21)CN(C(=O)Nc1ccc(F)c(Cl)c1)CC4] | N-(3-Chloro-4-fluorophenyl)-1- methyl-4,5,6,9,10,12- hexahydropyrazolo[3,4- c]pyrido[4′,3′:3,4]pyrazolo[1,5- a]azepine-11(1H)-carboxamide; |
| 31 | 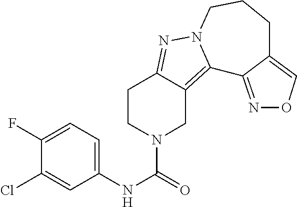 [SMILES: O=C(Nc1ccc(F)c(Cl)c1)N1CCc2nn3c(c2C1)-c1nocc1CCC3] | N-(3-Chloro-4-fluorophenyl)- 5,6,9,10-tetrahydro-4H- isoxazolo[3,4-c]pyrido [4′,3′:3,4]pyrazolo[1,5- a]azepine-11(12H)-carboxamide; |
| 32 | 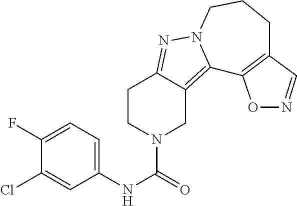 [SMILES: O=C(Nc1ccc(F)c(Cl)c1)N1CCc2nn3c(c2C1)-c1oncc1CCC3] | N-(3 Chloro-4-fluorophenyl)- 5,6,9,10-tetrahydro-4H- isoxazolo[5,4-c]pyrido [4′,3′:3,4]pyrazolo[1,5- a]azepine-11(12H)-carboxamide; |
| 33 | 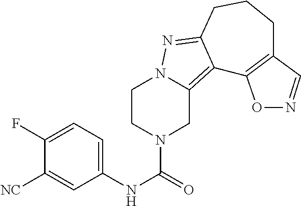 [SMILES: N#Cc1cc(NC(=O)N2CCn3nc4c(c3C2)-c2oncc2CCC4)ccc1F] | N-(3-Cyano-4-fluorophenyl)- 5,6,9,10-tetrahydro-4H- isoxazolo[5″,4″:3′,4′]cyclohepta [1′,2′:3,4]pyrazolo[1,5- a]pyrazine-11(12H)- carboxamide; |
| 34 | 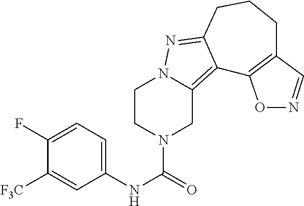 [SMILES: O=C(Nc1ccc(F)c(C(F)(F)F)c1)N1CCn2nc3c(c2C1)-c1oncc1CCC3] | N-(3-Cyano-4-fluorophenyl)- 5,6,9,10-tetrahydro-4H- isoxazolo[5″,4″:3′,4′]cyclohepta [1′,2′:3,4]pyrazolo[1,5- a]pyrazine-11(12H)- carboxamide; |
| 35 | 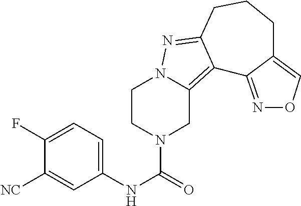 [SMILES: N#Cc1cc(NC(=O)N2CCn3nc4c(c3C2)-c2nocc2CCC4)ccc1F] | N-(3-Cyano-4-fluorophenyl)- 5,6,9,10-tetrahydro-4H- isoxazolo[3″,4″:3′,4′]cyclohepta [1′,2′:3,4]pyrazolo[1,5- a]pyrazine-11(12H)- carboxamide; and |
| 36 | 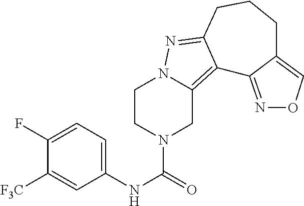 [SMILES: O=C(Nc1ccc(F)c(C(F)(F)F)c1)N1CCn2nc3c(c2C1)-c1nocc1CCC3] | N-(4-Fluoro-3- (trifluoromethyl)phenyl)- 5,6,9,10-tetrahydro-4H- isoxazolo[3″,4″:3′,4′]cyclohepta [1′,2′:3,4]pyrazolo[1,5- a]pyrazine-11(12H)- carboxamide; |

and pharmaceutically acceptable salts, N-oxides, or solvates thereof.

### Pharmaceutical Compositions

Also disclosed herein are pharmaceutical compositions comprising

(A) at least one compound of Formula (I):

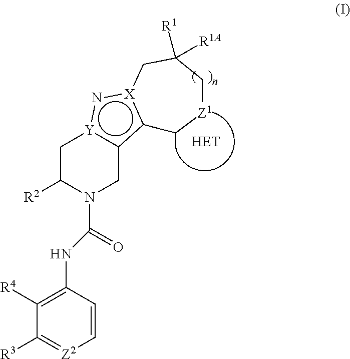

wherein
R¹ is independently selected from the group consisting of: hydrogen, C₁₋₄alkyl, hydroxy, hydroxymethyl, (2,2-difluoroethoxy)methyl, OC₁₋₄alkyl, and fluoro;
R^{1A} is independently hydrogen or taken together with R¹ to form methylenyl;
n is an integer that is 0, 1, or 2;
R² is independently selected from the group consisting of: hydrogen and C₁₋₆alkyl;
R³ is selected from the group consisting of: Cl, CN, and C₁₋₄haloalkyl;
R⁴ is H, or F;
HET is a 5- or 6-membered heteroaryl, optionally independently substituted with one to two substituents selected from C₁₋₄alkyl, bromo, chloro, fluoro, and hydroxy(C₁₋₄)alkyl;
X and Y are each independently selected from: N or C, such that only one of X and Y is N in any instance;
Z¹ is N or C; and
Z² is N or CF;
   and pharmaceutically acceptable salts, stereoisomers, isotopic variants, N-oxides or solvates of compounds of Formula (I); and

(B) at least one pharmaceutically acceptable excipient.

An embodiment of the present disclosure is a pharmaceutical composition comprising at least one pharmaceutically acceptable excipient and at least one compound selected from the group consisting of:

N-(3-Cyano-4-fluorophenyl)-5-methylene-5,6,9,10-tetrahydro-4H-isoxazolo[3,4-
c]pyrido[4′,3′:3,4]pyrazolo[1,5-a]azepine-11(12H)-carboxamide;
N-(4-Fluoro-3-(trifluoromethyl)phenyl)-5-methylene-5,6,9,10-tetrahydro-4H-
isoxazolo[3,4-c]pyrido[4′,3′:3,4]pyrazolo[1,5-a]azepine-11(12H)-carboxamide;
N-(3-Cyano-4-fluorophenyl)-5-(hydroxymethyl)-5,6,9,10-tetrahydro-4H-
isoxazolo[3,4-c]pyrido[4′,3′:3,4]pyrazolo[1,5-a]azepine-11(12H)-carboxamide;
N-(4-Fluoro-3-(trifluoromethyl)phenyl)-5-(hydroxymethyl)-5,6,9,10-tetrahydro-4H-
isoxazolo[3,4-c]pyrido[4′,3′:3,4]pyrazolo[1,5-a]azepine-11(12H)-carboxamide;
(5S*)-N-(3-Cyano-4-fluorophenyl)-5-((2,2-difluoroethoxy)methyl)-5,6,9,10-
tetrahydro-4H-isoxazolo[3,4-c]pyrido[4′,3′:3,4]pyrazolo[1,5-a]azepine-11(12H)-
carboxamide;
(5S*)-5-((2,2-Difluoroethoxy)methyl)-N-(4-fluoro-3-(trifluoromethyl)phenyl)-
5,6,9,10-tetrahydro-4H-isoxazolo[3,4-c]pyrido[4′,3′:3,4]pyrazolo[1,5-a]azepine-
11(12H)-carboxamide;
(5R*)-N-(3-Cyano-4-fluorophenyl)-5-((2,2-difluoroethoxy)methyl)-5,6,9,10-
tetrahydro-4H-isoxazolo[3,4-c]pyrido[4′,3′:3,4]pyrazolo[1,5-a]azepine-11(12H)-
carboxamide;
(5R*)-5-((2,2-Difluoroethoxy)methyl)-N-(4-fluoro-3-(trifluoromethyl)phenyl)-
5,6,9,10-tetrahydro-4H-isoxazolo[3,4-c]pyrido[4′,3′:3,4]pyrazolo[1,5-a]azepine-
11(12H)-carboxamide;
N-(3-Cyano-4-fluorophenyl)-5-methylene-5,6,9,10-tetrahydro-4H-isoxazolo[5,4-
c]pyrido[4′,3′:3,4]pyrazolo[1,5-a]azepine-11(12H)-carboxamide;
N-(4-Fluoro-3-(trifluoromethyl)phenyl)-5-methylene-5,6,9,10-tetrahydro-4H-
isoxazolo[5,4-c]pyrido[4′,3′:3,4]pyrazolo[1,5-a]azepine-11(12H)-carboxamide;
N-(3-Cyano-4-fluorophenyl)-5-hydroxy-5,6,9,10-tetrahydro-4H-isoxazolo[3,4-
c]pyrido[4′,3′:3,4]pyrazolo[1,5-a]azepine-11(12H)-carboxamide;
N-(4-Fluoro-3-(trifluoromethyl)phenyl)-5-methyl-5,6,9,10-tetrahydro-4H-
isoxazolo[3,4-c]pyrido[4′,3′:3,4]pyrazolo[1,5-a]azepine-11(12H)-carboxamide;
N-(3-Cyano-4-fluorophenyl)-5-methyl-5,6,9,10-tetrahydro-4H-isoxazolo[3,4-
c]pyrido[4′,3′:3,4]pyrazolo[1,5-a]azepine-11(12H)-carboxamide;
(10R)-N-(3-Cyano-4-fluorophenyl)-10-methyl-5,6,9,10-tetrahydro-4H-isoxazolo[5,4-
c]pyrido[4′,3′:3,4]pyrazolo[1,5-a]azepine-11(12H)-carboxamide;
(10R)-N-(4-Fluoro-3-(trifluoromethyl)phenyl)-10-methyl-5,6,9,10-tetrahydro-4H-
isoxazolo[5,4-c]pyrido[4′,3′:3,4]pyrazolo[1,5-a]azepine-11(12H)-carboxamide;
(11R)-N-(3-Cyano-4-fluorophenyl)-11-methyl-6,7,10,11-tetrahydro-5H-pyrido[2,3-
c]pyrido[4′,3′:3,4]pyrazolo[1,5-a]azepine-12(13H)-carboxamide;
(11R)-N-(4-Fluoro-3-(trifluoromethyl)phenyl)-11-methyl-6,7,10,11-tetrahydro-5H-
pyrido[2,3-c]pyrido[4′,3′:3,4]pyrazolo[1,5-a]azepine-12(13H)-carboxamide;
(10R)-N-(3-Cyano-4-fluorophenyl)-10-methyl-5,6,9,10-tetrahydro-4H-isoxazolo[3,4-
c]pyrido[4′,3′:3,4]pyrazolo[1,5-a]azepine-11(12H)-carboxamide;
(10R)-N-(4-Fluoro-3-(trifluoromethyl)phenyl)-10-methyl-5,6,9,10-tetrahydro-4H-
isoxazolo[3,4-c]pyrido[4′,3′:3,4]pyrazolo[1,5-a]azepine-11(12H)-carboxamide;
N-(3-Chloro-4-fluorophenyl)-6,7,10,11-tetrahydro-5H-pyrido[4′,3′:3,4]pyrazolo[1,5-
a][1,2,4]triazolo[3,4-c][1,4]diazepine-12(13H)-carboxamide;
N-(3-Chloro-4-fluorophenyl)-3-methyl-6,7,10,11-tetrahydro-5H-pyrido[4′,3′:3,4]-
pyrazolo[1,5-a][1,2,4]triazolo[3,4-c][1,4]diazepine-12(13H)-carboxamide;
(11R)-N-(3-Chloro-4-fluorophenyl)-11-methyl-6,7,10,11-tetrahydro-5H-
pyrido[4′,3′:3,4]pyrazolo[1,5-a][1,2,4]triazolo[3,4-c][1,4]diazepine-12(13H)-
carboxamide;
(11R)-N-(3-Chloro-4-fluorophenyl)-11-methyl-6,7,10,11-tetrahydro-5H-
pyrido[4′,3′:3,4]pyrazolo[1,5-a][1,2,4]triazolo[3,4-c][1,4]diazepine-12(13H)-
carboxamide;
N-(3-Cyano-4-fluorophenyl)-6,7,10,11-tetrahydro-5H-pyridazino[3,4-
c]pyrido[4′,3′:3,4]pyrazolo[1,5-a]azepine-12(13H)-carboxamide;
N-(3-Chloro-4-fluorophenyl)-4,5,6,9,10,12-hexahydropyrazolo[3,4-
c]pyrido[4′,3′:3,4]pyrazolo[1,5-a]azepine-11(2H)-carboxamide;
N-(3-Cyano-4-fluorophenyl)-4,5,6,9,10,12-hexahydropyrazolo[3,4-
c]pyrido[4′,3′:3,4]pyrazolo[1,5-a]azepine-11(2H)-carboxamide;
N-(3-Cyano-4-fluorophenyl)-6,7,10,11-tetrahydro-5H-pyrido[2,3-
c]pyrido[4′,3′:3,4]pyrazolo[1,5-a]azepine-12(13H)-carboxamide;
N-(4Fluoro-3-(trifluoromethyl)phenyl)-6,7,10,11-tetrahydro-5H-pyrido[2,3-
c]pyrido[4′,3′:3,4]pyrazolo[1,5-a]azepine-12(13H)-carboxamide;
N-(3-Chloro-4-fluorophenyl)-2-methyl-4,5,6,9,10,12-hexahydropyrazolo[3,4-
c]pyrido[4′,3′:3,4]pyrazolo[1,5-a]azepine-11(2H)-carboxamide;
N-(3-Chloro-4-fluorophenyl)-1-methyl-4,5,6,9,10,12-hexahydropyrazolo[3,4-
c]pyrido[4′,3′:3,4]pyrazolo[1,5-a]azepine-11(1H)-carboxamide;
N-(3-Chloro-4-fluorophenyl)-5,6,9,10-tetrahydro-4H-isoxazolo[3,4-c]pyrido
[4′,3′:3,4]pyrazolo[1,5-a]azepine-11(12H)-carboxamide;
N-(3Chloro-4-fluorophenyl)-5,6,9,10-tetrahydro-4H-isoxazolo[5,4-c]pyrido
[4′,3′:3,4]pyrazolo[1,5-a]azepine-11(12H)-carboxamide;
N-(3-Cyano-4-fluorophenyl)-5,6,9,10-tetrahydro-4H-isoxazolo[5″,4″:3′,4′]-
cyclohepta[1′,2′:3,4]pyrazolo[1,5-a]pyrazine-11(12H)-carboxamide;
N-(3-Cyano-4-fluorophenyl)-5,6,9,10-tetrahydro-4H-isoxazolo[5″,4″:3′,4′]-
cyclohepta[1′,2′:3,4]pyrazolo[1,5-a]pyrazine-11(12H)-carboxamide;
N-(3-Cyano-4-fluorophenyl)-5,6,9,10-tetrahydro-4H-isoxazolo[3″,4″:3′,4′]-
cyclohepta[1′,2′:3,4]pyrazolo[1,5-a]pyrazine-11(12H)-carboxamide; and
N-(4-Fluoro-3-(trifluoromethyl)phenyl)-5,6,9,10-tetrahydro-4H-isoxazolo-
[3″,4″:3′,4′]cyclohepta[1′,2′:3,4]pyrazolo[1,5-a]pyrazine-11(12H)-carboxamide;

as well as any pharmaceutically acceptable salt, N-oxide or solvate of such compound, or any pharmaceutically acceptable prodrugs of such compound, or any pharmaceutically active metabolite of such compound.

In embodiments, the pharmaceutical composition comprises at least one additional active or therapeutic agent. Additional active therapeutic agents may include, for example, an anti-HBV agent such as an HBV polymerase inhibitor, interferon, viral entry inhibitor, viral maturation inhibitor, capsid assembly modulator, reverse transcriptase inhibitor, immunomodulatory agent such as a TLR-agonist, or any other agents that affects the HBV life cycle and/or the consequences of HBV infection. The active agents of the present disclosure are used, alone or in combination with one or more additional active agents, to formulate pharmaceutical compositions of the present disclosure.

As used herein, the term “composition” or “pharmaceutical composition” refers to a mixture of at least one compound useful within the present disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the present disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the present disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the patient. Supplementary active compounds may also be incorporated into the compositions. The “pharmaceutically acceptable carrier” may further include a pharmaceutically acceptable salt of the compound useful within the present disclosure. Other additional ingredients that may be included in the pharmaceutical compositions used in the practice of the present disclosure are known in the art and described, for example in Remington's Pharmaceutical Sciences (Genaro, Ed., Mack Publishing Co., 1985, Easton, Pa.), which is incorporated herein by reference.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the present disclosure can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets may include a compound according to the present disclosure mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, compounds of the present disclosure may be mixed with a solid, semi-solid, or liquid diluent. Soft gelatin capsules may be prepared by mixing the compound of the present disclosure with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this present disclosure may also be administered by non-oral routes. For example, the compositions may be formulated for rectal administration as a suppository. For parenteral use, including intravenous, intramuscular, intraperitoneal, or subcutaneous routes, the compounds of the present disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 g/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the present disclosure may utilize a patch formulation to affect transdermal delivery.

Compounds of the present disclosure may alternatively be administered in methods of this present disclosure by inhalation, via the nasal or oral routes, e.g., in a spray formulation also containing a suitable carrier.

### Methods of Use

The disclosed compounds are useful in the treatment and prevention of HBV infection in a subject such as a human subject.

In a non-limiting aspect, these compounds may (i) modulate or disrupt HBV assembly and other HBV core protein functions necessary for HBV replication or the generation of infectious particles, (ii) inhibit the production of infectious virus particles or infection, or (iii) interact with HBV capsid to effect defective viral particles with reduced infectivity or replication capacity acting as capsid assembly modulators. In particular, and without being bound to any particular mechanism of action, it is believed that the disclosed compounds are useful in HBV treatment by disrupting, accelerating, reducing, delaying and/or inhibiting normal viral capsid assembly and/or disassembly of immature or mature particles, thereby inducing aberrant capsid morphology leading to antiviral effects such as disruption of virion assembly and/or disassembly, virion maturation, virus egress and/or infection of target cells. The disclosed compounds may act as a disruptor of capsid assembly interacting with mature or immature viral capsid to perturb the stability of the capsid, thus affecting its assembly and/or disassembly. The disclosed compounds may perturb protein folding and/or salt bridges required for stability, function and/or normal morphology of the viral capsid, thereby disrupting and/or accelerating capsid assembly and/or disassembly. The disclosed compounds may bind capsid and alter metabolism of cellular polyproteins and precursors, leading to abnormal accumulation of protein monomers and/or oligomers and/or abnormal particles, which causes cellular toxicity and death of infected cells. The disclosed compounds may cause failure of the formation of capsids of optimal stability, affecting efficient uncoating and/or disassembly of viruses (e.g., during infectivity). The disclosed compounds may disrupt and/or accelerate capsid assembly and/or disassembly when the capsid protein is immature. The disclosed compounds may disrupt and/or accelerate capsid assembly and/or disassembly when the capsid protein is mature. The disclosed compounds may disrupt and/or accelerate capsid assembly and/or disassembly during viral infectivity which may further attenuate HBV viral infectivity and/or reduce viral load. The disruption, acceleration, inhibition, delay and/or reduction of capsid assembly and/or disassembly by the disclosed compounds may eradicate the virus from the host organism. Eradication of HBV from a subject by the disclosed compounds advantageously obviates the need for chronic long-term therapy and/or reduces the duration of long-term therapy.

An additional embodiment of the present disclosure is a method of treating a subject suffering from an HBV infection, comprising administering to a subject in need of such treatment an effective amount of at least one compound of Formula (I).

In another aspect, provided herein is a method of reducing the viral load associated with an HBV infection in an individual in need thereof, comprising administering to the individual a therapeutically effective amount of a compound of Formula (I), or a pharmaceutically acceptable salt thereof.

In another aspect, provided herein is a method of reducing reoccurrence of an HBV infection in an individual in need thereof, comprising administering to the individual a therapeutically effective amount of a compound of Formula (I), or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (I), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (I), or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (I), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (I), or a pharmaceutically acceptable salt thereof.

In embodiments, the disclosed compounds are suitable for monotherapy. In embodiments, the disclosed compounds are effective against natural or native HBV strains.

In embodiments, the disclosed compounds are effective against HBV strains resistant to currently known drugs.

In another embodiment, the compounds provided herein can be used in methods of modulating (e.g., inhibiting or disrupting) the activity, stability, function, and viral replication properties of HBV cccDNA.

In yet another embodiment, the compounds of the present disclosure can be used in methods of diminishing or preventing the formation of HBV cccDNA.

In another embodiment, the compounds provided herein can be used in methods of modulating (e.g., inhibiting or disrupting) the activity of HBV cccDNA.

In yet another embodiment, the compounds of the present disclosure can be used in methods of diminishing the formation of HBV cccDNA.

In another embodiment, the disclosed compounds can be used in methods of modulating, inhibiting, or disrupting the generation or release of HBV RNA particles from within the infected cell.

In a further embodiment, the total burden (or concentration) of HBV RNA particles is modulated. In a preferred embodiment, the total burden of HBV RNA is diminished.

In another embodiment, the methods provided herein reduce the viral load in the individual to a greater extent or at a faster rate compared to the administering of a compound selected from the group consisting of an HBV polymerase inhibitor, interferon, viral entry inhibitor, viral maturation inhibitor, distinct capsid assembly modulator, antiviral compounds of distinct or unknown mechanism, and any combination thereof.

In another embodiment, the methods provided herein cause a lower incidence of viral mutation and/or viral resistance than the administering of a compound selected from the group consisting of an HBV polymerase inhibitor, interferon, viral entry inhibitor, viral maturation inhibitor, distinct capsid assembly modulator, antiviral compounds of distinct or unknown mechanism, and combination thereof.

In another embodiment, the methods provided herein further comprise administering to the individual at least one HBV vaccine, a nucleoside HBV inhibitor, an interferon or any combination thereof.

In an aspect, provided herein is a method of treating an HBV infection in an individual in need thereof, comprising reducing the HBV viral load by administering to the individual a therapeutically effective amount of a compound of Formula (I), or a pharmaceutically acceptable salt thereof, alone or in combination with a reverse transcriptase inhibitor; and further administering to the individual a therapeutically effective amount of HBV vaccine.

An additional embodiment of the present disclosure is a method of treating a subject suffering from an HBV infection, comprising administering to a subject in need of such treatment an effective amount of at least one compound of Formula (I).

In another aspect, provided herein is a method of reducing the viral load associated with an HBV infection in an individual in need thereof, comprising administering to the individual a therapeutically effective amount of a compound of Formula (I), or a pharmaceutically acceptable salt thereof.

In another aspect, provided herein is a method of reducing reoccurrence of an HBV infection in an individual in need thereof, comprising administering to the individual a therapeutically effective amount of a compound of Formula (I), or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (I), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (I), or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (I), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (I), or a pharmaceutically acceptable salt thereof.

In an embodiment, the methods provided herein further comprise monitoring the HBV viral load of the subject, wherein the method is carried out for a period of time such that the HBV virus is undetectable.

### Combinations

Provided herein are combinations of one or more of the disclosed compounds with at least one additional therapeutic agent. In embodiments, the methods provided herein can further comprise administering to the individual at least one additional therapeutic agent. In embodiments, the disclosed compounds are suitable for use in combination therapy. The compounds of the present disclosure may be useful in combination with one or more additional compounds useful for treating HBV infection. These additional compounds may comprise compounds of the present disclosure or compounds known to treat, prevent, or reduce the symptoms or effects of HBV infection.

In an exemplary embodiment, additional active ingredients are those that are known or discovered to be effective in the treatment of conditions or disorders involved in HBV infection, such as another HBV capsid assembly modulator or a compound active against another target associated with the particular condition or disorder involved in HBV infection, or the HBV infection itself. The combination may serve to increase efficacy (e.g., by including in the combination a compound potentiating the potency or effectiveness of an active agent according to the present disclosure), decrease one or more side effects, or decrease the required dose of the active agent according to the present disclosure. In a further embodiment, the methods provided herein allow for administering of the at least one additional therapeutic agent at a lower dose or frequency as compared to the administering of the at least one additional therapeutic agent alone that is required to achieve similar results in prophylactically treating an HBV infection in an individual in need thereof.

Such compounds include but are not limited to HBV combination drugs, HBV vaccines, HBV DNA polymerase inhibitors, immunomodulatory agents, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and any other agent that affects the HBV life cycle and/or affect the consequences of HBV infection or combinations thereof.

In embodiments, the compounds of the present disclosure may be used in combination with an HBV polymerase inhibitor, immunomodulatory agents, interferon such as pegylated interferon, viral entry inhibitor, viral maturation inhibitor, capsid assembly modulator, reverse transcriptase inhibitor, a cyclophilin/TNF inhibitor, immunomodulatory agent such as a TLR-agonist, an HBV vaccine, and any other agent that affects the HBV life cycle and/or affect the consequences of HBV infection or combinations thereof.

In particular, the compounds of the present disclosure may be used in combination with one or more agents (or a salt thereof) selected from the group consisting of

HBV reverse transcriptase inhibitors, and DNA and RNA polymerase inhibitors, including but not limited to: lamivudine (3TC, Zeffix, Heptovir, Epivir, and Epivir-HBV), entecavir (Baraclude, Entavir), adefovir dipivoxil (Hepsara, Preveon, bis-POM PMEA), tenofovir disoproxil fumarate (Viread, TDF or PMPA);

interferons, including but not limited to interferon alpha (IFN-α), interferon beta (IFN-β), interferon lambda (IFN-λ), and interferon gamma (IFN-γ);

viral entry inhibitors;

viral maturation inhibitors;

literature-described capsid assembly modulators, such as, but not limited to BAY 41-4109;

reverse transcriptase inhibitor;

an immunomodulatory agent such as a TLR-agonist; and

agents of distinct or unknown mechanism, such as but not limited to AT-61 ((E)-N-(1-chloro-3-oxo-1-phenyl-3-(piperidin-1-yl)prop-1-en-2-yl)benzamide), AT-130 ((E)-N-(1-bromo-1-(2-methoxyphenyl)-3-oxo-3-(piperidin-1-yl)prop-1-en-2-yl)-4-nitrobenzamide), and similar analogs.

In embodiments, the additional therapeutic agent is an interferon. The term “interferon” or “IFN” refers to any member the family of highly homologous species-specific proteins that inhibit viral replication and cellular proliferation, and modulate immune response. Human interferons are grouped into three classes; Type I, which include interferon-alpha (IFN-α), interferon-beta (IFN-β), and interferon-omega (IFN-ω), Type II, which includes interferon-gamma (IFN-γ), and Type III, which includes interferon-lambda (IFN-λ). Recombinant forms of interferons that have been developed and are commercially available are encompassed by the term “interferon” as used herein. Subtypes of interferons, such as chemically modified or mutated interferons, are also encompassed by the term “interferon” as used herein. Chemically modified interferons include pegylated interferons and glycosylated interferons. Examples of interferons also include, but are not limited to, interferon-alpha-2a, interferon-alpha-2b, interferon-alpha-n1, interferon-beta-1a, interferon-beta-1b, interferon-lamda-1, interferon-lamda-2, and interferon-lamda-3. Examples of pegylated interferons include pegylated interferon-alpha-2a and pegylated interferon alpha-2b.

Accordingly, in one embodiment, the compounds of Formula I, can be administered in combination with an interferon selected from the group consisting of interferon alpha (IFN-α), interferon beta (IFN-β), interferon lambda (IFN-λ), and interferon gamma (IFN-γ). In one specific embodiment, the interferon is interferon-alpha-2a, interferon-alpha-2b, or interferon-alpha-n1. In another specific embodiment, the interferon-alpha-2a or interferon-alpha-2b is pegylated. In a preferred embodiment, the interferon-alpha-2a is pegylated interferon-alpha-2a (PEGASYS).

In another embodiment, the additional therapeutic agent is selected from immune modulator or immune stimulator therapies, which includes biological agents belonging to the interferon class.

Further, the additional therapeutic agent may be an agent that disrupts the function of other essential viral protein(s) or host proteins required for HBV replication or persistence.

In another embodiment, the additional therapeutic agent is an antiviral agent that blocks viral entry or maturation or targets the HBV polymerase such as nucleoside or nucleotide or non-nucleos(t)ide polymerase inhibitors. In a further embodiment of the combination therapy, the reverse transcriptase inhibitor and/or DNA and/or RNA polymerase inhibitor is Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

In an embodiment, the additional therapeutic agent is an immunomodulatory agent that induces a natural, limited immune response leading to induction of immune responses against unrelated viruses. In other words, the immunomodulatory agent can affect maturation of antigen presenting cells, proliferation of T-cells and cytokine release (e.g., IL-12, IL-18, IFN-alpha, -beta, and -gamma and TNF-alpha among others).

In a further embodiment, the additional therapeutic agent is a TLR modulator or a TLR agonist, such as a TLR-7 agonist or TLR-9 agonist. In further embodiment of the combination therapy, the TLR-7 agonist is selected from the group consisting of SM360320 (9-benzyl-8-hydroxy-2-(2-methoxy-ethoxy)adenine) and AZD 8848 (methyl [3-({[3-(6-amino-2-butoxy-8-oxo-7,8-dihydro-9H-purin-9-yl)propyl][3-(4-morpholinyl)propyl]amino}-methyl)phenyl]acetate).

In any of the methods provided herein, the method may further comprise administering to the individual at least one HBV vaccine, a nucleoside HBV inhibitor, an interferon or any combination thereof. In an embodiment, the HBV vaccine is at least one of RECOMBIVAX HB, ENGERIX-B, ELOVAC B, GENEVAC-B, or SHANVAC B.

In another aspect, provided herein is method of treating an HBV infection in an individual in need thereof, comprising reducing the HBV viral load by administering to the individual a therapeutically effective amount of a compound of the present disclosure alone or in combination with a reverse transcriptase inhibitor; and further administering to the individual a therapeutically effective amount of HBV vaccine. The reverse transcriptase inhibitor may be one of Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

For any combination therapy described herein, synergistic effect may be calculated, for example, using suitable methods such as the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Definitions

Listed below are definitions of various terms used to describe this present disclosure. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the applicable art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include,” “includes,” and “included,” is not limiting.

As used in the specification and in the claims, the term “comprising” can include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

All ranges disclosed herein are inclusive of the recited endpoint and independently combinable (for example, the range of “from 50 mg to 300 mg” is inclusive of the endpoints, 50 mg and 300 mg, and all the intermediate values). The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “substantially,” cannot be limited to the precise value specified, in some cases. In at least some instances, the approximating language can correspond to the precision of an instrument for measuring the value.

The term “alkyl” refers to a straight- or branched-chain alkyl group having from 1 to 12 carbon atoms in the chain. Examples of alkyl groups include methyl (Me, which also may be structurally depicted by the symbol, “/”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. The term C₁₋₄alkyl as used here refers to a straight- or branched-chain alkyl group having from 1 to 4 carbon atoms in the chain. The term C₁₋₆alkyl as used here refers to a straight- or branched-chain alkyl group having from 1 to 6 carbon atoms in the chain.

The term “heteroaryl” refers to an aromatic monocyclic or bicyclic aromatic ring system having 5 to 10 ring members and which contains carbon atoms and from 1 to 4 heteroatoms independently selected from the group consisting of N, O, and S. Included within the term heteroaryl are aromatic rings of 5 or 6 members wherein the ring consists of carbon atoms and has at least one heteroatom member. Suitable heteroatoms include nitrogen, oxygen, and sulfur. In the case of 5 membered rings, the heteroaryl ring preferably contains one member of nitrogen, oxygen or sulfur and, in addition, up to 3 additional nitrogens. In the case of 6 membered rings, the heteroaryl ring preferably contains from 1 to 3 nitrogen atoms. For the case wherein the 6 membered ring has 3 nitrogens, at most 2 nitrogen atoms are adjacent. Examples of heteroaryl groups include furyl, thienyl, pyrrolyl, oxazolyl, thiazolyl, imidazolyl, pyrazolyl, oxazolyl, thiazolyl, oxadiazolyl, triazolyl, thiadiazolyl, pyridinyl, pyridazinyl, pyrimidinyl, pyrazinyl, indolyl, isoindolyl, benzofuryl, benzothienyl, indazolyl, benzimidazolyl, benzothiazolyl, benzoxazolyl, benzisoxazolyl, benzothiadiazolyl, benzotriazolyl, quinolinyl, isoquinolinyl and quinazolinyl. Unless otherwise noted, the heteroaryl is attached to its pendant group at any heteroatom or carbon atom that results in a stable structure.

Those skilled in the art will recognize that the species of heteroaryl groups listed or illustrated above are not exhaustive, and that additional species within the scope of these defined terms may also be selected.

The term “cyano” refers to the group —CN.

The term “halo” represents chloro, fluoro, bromo or iodo.

The term “perhaloalkyl” or “haloalkyl” refers to a straight- or branched-chain alkyl group having from 1 to 6 carbon atoms in the chain optionally substituting hydrogens with halogens. The term “C₁₋₄haloalkyl” as used here refers to a straight- or branched-chain alkyl group having from 1 to 4 carbon atoms in the chain, optionally substituting hydrogens with halogens. The term “C₁₋₆haloalkyl” as used here refers to a straight- or branched-chain alkyl group having from 1 to 6 carbon atoms in the chain, optionally substituting hydrogens with halogens. Examples of “perhaloalkyl”, “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (—CF(CF₃)₂), and groups that in light of the ordinary skill in the art and the teachings provided herein would be considered equivalent to any one of the foregoing examples.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

The terms “para”, “meta”, and “ortho” have the meanings as understood in the art.

Thus, for example, a fully substituted phenyl group has substituents at both “ortho” (o) positions adjacent to the point of attachment of the phenyl ring, both “meta” (m) positions, and the one “para” (p) position across from the point of attachment. To further clarify the position of substituents on the phenyl ring, the 2 different ortho positions will be designated as ortho and ortho′ and the 2 different meta positions as meta and meta′ as illustrated below.

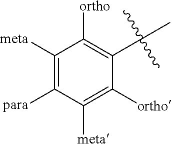

When referring to substituents on a pyridyl group, the terms “para”, “meta”, and “ortho” refer to the placement of a substituent relative to the point of attachment of the pyridyl ring. For example, the structure below is described as 3-pyridyl with the X¹ substituent in the ortho position, the X² substituent in the meta position, and X³ substituent in the para position:

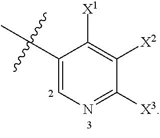

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

The terms “buffered” solution or “buffer” solution are used herein interchangeably according to their standard meaning. Buffered solutions are used to control the pH of a medium, and their choice, use, and function is known to those of ordinary skill in the art. See, for example, G. D. Considine, ed., Van Nostrand's Encyclopedia of Chemistry, p. 261, 5^{th} ed. (2005), describing, inter alia, buffer solutions and how the concentrations of the buffer constituents relate to the pH of the buffer. For example, a buffered solution is obtained by adding MgSO₄ and NaHCO₃ to a solution in a 10:1 w/w ratio to maintain the pH of the solution at about 7.5.

Any formula given herein is intended to represent compounds having structures depicted by the structural formula as well as certain variations or forms. In particular, compounds of any formula given herein may have asymmetric centers and therefore exist in different enantiomeric forms. All optical isomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers.

It is also to be understood that compounds that have the same molecular formula but differ in the nature or sequence of bonding of their atoms or the arrangement of their atoms in space are termed “isomers.”

Stereoisomers that are not mirror images of one another are termed “diastereomers” and those that are non-superimposable mirror images of each other are termed “enantiomers.” When a compound has an asymmetric center, for example, it is bonded to four different groups, and a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either an individual enantiomer or as a mixture thereof. A mixture containing equal proportions of the enantiomers is called a “racemic mixture.”

“Tautomers” refer to compounds that are interchangeable forms of a particular compound structure, and that vary in the displacement of hydrogen atoms and electrons. Thus, two structures may be in equilibrium through the movement of 7 electrons and an atom (usually H). For example, enols and ketones are tautomers because they are rapidly interconverted by treatment with either acid or base. Another example of tautomerism is the aci- and nitro-forms of phenyl nitromethane, that are likewise formed by treatment with acid or base.

Tautomeric forms may be relevant to the attainment of the optimal chemical reactivity and biological activity of a compound of interest.

The compounds of this present disclosure may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Certain examples contain chemical structures that are depicted as an absolute enantiomer but are intended to indicate enantiopure material that is of unknown configuration. In these cases (R*) or (S*) is used in the name to indicate that the absolute stereochemistry of the corresponding stereocenter is unknown. Thus, a compound designated as (R*) refers to an enantiopure compound with an absolute configuration of either (R) or (S). In cases where the absolute stereochemistry has been confirmed, the structures are named using (R) and (S).

The symbols [symbol] and [symbol] are used as meaning the same spatial arrangement in chemical structures shown herein. Analogously, the symbols [symbol] and [symbol] m are used as meaning the same spatial arrangement in chemical structures shown herein.

Additionally, any formula given herein is intended to refer also to hydrates, solvates, and polymorphs of such compounds, and mixtures thereof, even if such forms are not listed explicitly. Certain compounds of Formula (I), or pharmaceutically acceptable salts of compounds of Formula (I), may be obtained as solvates. Solvates include those formed from the interaction or complexation of compounds of the present disclosure with one or more solvents, either in solution or as a solid or crystalline form. In some embodiments, the solvent is water and the solvates are hydrates. In addition, certain crystalline forms of compounds of Formula (I), or pharmaceutically acceptable salts of compounds of Formula (I) may be obtained as co-crystals. In certain embodiments of the present disclosure, compounds of Formula (I) were obtained in a crystalline form. In other embodiments, crystalline forms of compounds of Formula (I) were cubic in nature. In other embodiments, pharmaceutically acceptable salts of compounds of Formula (I) were obtained in a crystalline form. In still other embodiments, compounds of Formula (I) were obtained in one of several polymorphic forms, as a mixture of crystalline forms, as a polymorphic form, or as an amorphous form. In other embodiments, compounds of Formula (I) convert in solution between one or more crystalline forms and/or polymorphic forms.

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

In another example, a zwitterionic compound is encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI.27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion ⁺H₃NCH₂COO⁻. Zwitterions, zwitterionic compounds, inner salts and dipolar ions in the known and well established meanings of these terms are within the scope of this present disclosure, as would in any case be so appreciated by those of ordinary skill in the art. Because there is no need to name each and every embodiment that would be recognized by those of ordinary skill in the art, no structures of the zwitterionic compounds that are associated with the compounds of this present disclosure are given explicitly herein. They are, however, part of the embodiments of this present disclosure. No further examples in this regard are provided herein because the interactions and transformations in a given medium that lead to the various forms of a given compound are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorus, sulfur, fluorine, chlorine, and iodine such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Such isotopically labeled compounds are useful in metabolic studies (preferably with ¹⁴C), reaction kinetic studies (with, for example deuterium (i.e., D or ²H); or tritium (i.e., T or ³H)), detection or imaging techniques such as positron emission tomography (PET) or single-photon emission computed tomography (SPECT) including drug or substrate tissue distribution assays, or in radioactive treatment of patients. In particular, an 18F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this present disclosure and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this present disclosure given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein. The foregoing convention given herein for substituents extends, when applicable, to members such as HET, R¹, R^{1A}, R², R³, R⁴, R^{a} R^{b}, PG, PG¹, n, X, Y, Z¹ and Z² and any other generic substituent symbol used herein.

Furthermore, when more than one assignment is given for any member or substituent, embodiments of this present disclosure comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this present disclosure for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to members such as HET, R¹, R^{1A}, R², R³, R⁴, R^{a} R^{b}, PG, PG¹, n, X, Y, Z¹ and Z² and any other generic substituent symbol used herein.

The nomenclature “Cᵢ₋ⱼ” with j>i, when applied herein to a class of substituents, is meant to refer to embodiments of this present disclosure for which each and every one of the number of carbon members, from i to j including i and j, is independently realized. By way of example, the term C₁₋₄ refers independently to embodiments that have one carbon member (C₁), embodiments that have two carbon members (C₂), embodiments that have three carbon members (C₃), and embodiments that have four carbon members (C₄).

The term Cₙ₋ₘalkyl refers to an aliphatic chain, whether straight or branched, with a total number N of carbon members in the chain that satisfies n≤N≤m, with m>n. Any disubstituent referred to herein is meant to encompass the various attachment possibilities when more than one of such possibilities are allowed. For example, reference to disubstituent -A−B—, where A≠B, refers herein to such disubstituent with A attached to a first substituted member and B attached to a second substituted member, and it also refers to such disubstituent with A attached to the second substituted member and B attached to the first substituted member.

The present disclosure includes also pharmaceutically acceptable salts of the compounds of Formula (I), preferably of those described above and of the specific compounds exemplified herein, and methods of treatment using such salts.

The term “pharmaceutically acceptable” means approved or approvable by a regulatory agency of Federal or a state government or the corresponding agency in countries other than the United States, or that is listed in the U. S. Pharmcopoeia or other generally recognized pharmacopoeia for use in animals, and more particularly, in humans.

A “pharmaceutically acceptable salt” is intended to mean a salt of a free acid or base of compounds represented by Formula (I) that are non-toxic, biologically tolerable, or otherwise biologically suitable for administration to the subject. It should possess the desired pharmacological activity of the parent compound. See, generally, G. S. Paulekuhn, et al., “Trends in Active Pharmaceutical Ingredient Salt Selection based on Analysis of the Orange Book Database”, J. Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002.

Examples of pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt.

The present disclosure also relates to pharmaceutically acceptable prodrugs of the compounds of Formula (I), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of the compounds of Formula (I), which may also be used in the methods of the present disclosure. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al., J Med Chem. 1997, 40, 2011-2016; Shan, et al., J Pharm Sci. 1997, 86 (7), 765-767; Bagshawe, Drug Dev Res. 1995, 34, 220-230; Bodor, Adv Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

As used herein, the term “composition” or “pharmaceutical composition” refers to a mixture of at least one compound provided herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound provided herein within or to the patient such that it can perform its intended function. Typically, such constructs are carried or transported from one organ, or portion of the body, to another organ, or portion of the body. Each carrier must be “acceptable” in the sense of being compatible with the other ingredients of the formulation, including the compound provided herein, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound provided herein, and are physiologically acceptable to the patient. Supplementary active compounds can also be incorporated into the compositions. The “pharmaceutically acceptable carrier” can further include a pharmaceutically acceptable salt of the compound provided herein. Other additional ingredients that can be included in the pharmaceutical compositions provided herein are known in the art and described, for example in Remington's Pharmaceutical Sciences (Genaro, Ed., Mack Publishing Co., 1985, Easton, Pa.), which is incorporated herein by reference.

The term “stabilizer,” as used herein, refers to polymers capable of chemically inhibiting or preventing degradation of a compound of Formula I. Stabilizers are added to formulations of compounds to improve chemical and physical stability of the compound.

The term “tablet,” as used herein, denotes an orally administrable, single-dose, solid dosage form that can be produced by compressing a drug substance or a pharmaceutically acceptable salt thereof, with suitable excipients (e.g., fillers, disintegrants, lubricants, glidants, and/or surfactants) by conventional tableting processes. The tablet can be produced using conventional granulation methods, for example, wet or dry granulation, with optional comminution of the granules with subsequent compression and optional coating. The tablet can also be produced by spray-drying.

As used herein, the term “capsule” refers to a solid dosage form in which the drug is enclosed within either a hard or soft soluble container or “shell.” The container or shell can be formed from gelatin, starch and/or other suitable substances.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

The term “combination,” “therapeutic combination,” “pharmaceutical combination,” or “combination product” as used herein refer to a non-fixed combination or a kit of parts for the combined administration where two or more therapeutic agents can be administered independently, at the same time or separately within time intervals, especially where these time intervals allow that the combination partners show a cooperative, e.g., synergistic, effect.

The term “modulators” include both inhibitors and activators, where “inhibitors” refer to compounds that decrease, prevent, inactivate, desensitize, or down-regulate HBV assembly and other HBV core protein functions necessary for HBV replication or the generation of infectious particles.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In one embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies and/or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity and/or is lethal to the virus.

As used herein, the term “treatment” or “treating,” is defined as the application or administration of a therapeutic agent, i.e., a compound of the present disclosure (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject or individual is human.

In treatment methods according to the present disclosure, an effective amount of a pharmaceutical agent according to the present disclosure is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

An example of a dose of a compound is from about 1 mg to about 2,500 mg. In some embodiments, a dose of a compound of the present disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

HBV infections that may be treated according to the disclosed methods include HBV genotype A, B, C, and/or D infections. However, in an embodiment, the methods disclosed may treat any HBV genotype (“pan-genotypic treatment”). HBV genotyping may be performed using methods known in the art, for example, INNO-LIPA® HBV Genotyping, Innogenetics N.V., Ghent, Belgium).

### EXAMPLES

Exemplary compounds useful in methods of the present disclosure will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Abbreviations and acronyms used herein include the following set forth in Table 2:

TABLE 2

| Term | Acronym |
|---|---|
| Acetonitrile | ACN or MeCN |
| Aqueous | aq |
| Atmosphere | atm |
| 9-Borabicyclo(3.3.1)nonane | 1,9-BBN |
| n-butyllithium | BuLi |
| tert-butyloxycarbonyl | Boc or BOC |
| Boron-dipyrromethene | BODIPY |
| Broad | br |
| Capsid assembly | CA |
| 1,8-Diazabicyclo[5.4.0]undec-7-ene | DBU |
| Doublet of doublets | dd |
| Dichloroethane | DCE |
| Dichloromethane | DCM |
| 2,3-Dichloro-5,6-dicyano-1,4-benzoquinone | DDQ |
| Dimethylacetamide | DMA |
| 4-Dimethylaminopyridine | DMAP |
| Dimethylformamide | DMF |
| Dimethylsulfoxide | DMSO |
| Deoxyribonucleic Acid | DNA |
| Diethyl ether | Ether, Et₂O |
| Diisopropylethylamine | DIEA |
| Ethyl Acetate | EtOAc, or EA |
| Ethanol | EtOH |
| Electrospray ionization | ESI |
| Normal-phasesilica gel chromatography | FCC |
| Grams | g |
| Hours | h or hr |
| Hepatitis B Virus | HBV |
| Acetic acid | HOAc |
| High-pressure liquid chromatography | HPLC |
| Hertz | Hz |
| hexamethyldisilazane | HMDS |
| hexamethylphosphoramide | HMPA |
| Isopropylmagnesiumchloride | iPrMgCl |
| Isopropyl alcohol | iPrOH, IPA |
| Potassium tert-butoxide | KOtBu |
| Lithium aluminumhydride | LAH |
| Liquid chromatography and mass spectrometry | LCMS |
| Lithium diisopropylamide | LDA |
| Lithium bis(trimethylsilyl)amide | LHMDS, LiHMDS |
| Molar | M |
| multiplet | m |
| Mass to charge ratio | m/z |
| Methanol | MeOH |
| Milligrams | mg |
| Megahertz | MHz |
| Minute | min |
| Milliliter | mL |
| Microliter | μL |
| Millimole | mmol |
| Micromole | μmol |
| Mass spectrometry | MS |
| Mesityl chloride | MsCl |
| Normal | N |
| Reverse phase | RP |
| Sodium bis(trimethylsilyl)amide | NaHMDS |
| Sodium acetate | NaOAc, AcONa |
| N-iodosuccinimide | NIS |
| N-methylmorpholine N-oxide | NMO |
| Nuclear magnetic resonance | NMR |
| Polymerase chain reaction | PCR |
| Petroleum ether | PE |
| 9-(2-Phosphonyl-methoxypropyly)adenine | PMPA |
| Parts per million | ppm |
| Precipitate | ppt |
| Pyridine | Py |
| Retention time | Rt |
| Ribonucleic Acid | RNA |
| Room temperature | rt |
| singlet | s |
| Saturated | sat |
| Supercritical Fluid Chromatography | SFC |
| Temperature | T |
| triplet | t |
| Tetra-n-butylammonium fluoride | TBAF |
| Tris(dimethylamino)methane | TDAM |
| Triethylamine | TEA |
| Trifluoroacetic acid | TFA |
| Tetrahydrofuran | THF |
| Thin layer chromatography | TLC |
| Toll-like receptor | TLR |
| Tumor necrosis factor | TNF |
| Tetrapropylammonium perruthenate | TPAP |
| Volumein milliliters of solvent per gram of substrate | V, or volumes |

### Synthesis

Exemplary compounds useful in methods of the present disclosure will now be described by reference to the illustrative synthetic schemes for their general preparation below and the specific examples to follow.

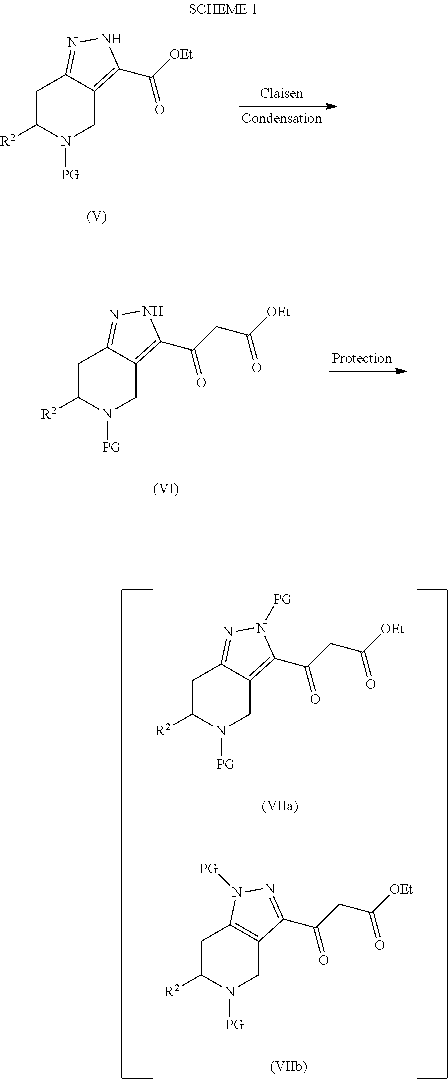

According to SCHEME 1, a compound of formula (V), where R² is H or C₁₋₆alkyl, and PG is BOC, undergoes a Claisen-type reaction or acylation with ethyl acetate; in the presence of a suitable base such as sodium hydride, potassium hydride, lithium diisopropylamide (LDA), lithium hexamethyldisilylamide (LHMDS), sodium bis(trimethylsilyl)amide (NaHMDS), potassium butoxide, and the like; preferably sodium bis(trimethylsilyl)amide (NaHMDS); in a suitable solvent such as tetrahydrofuran (THF), dioxane, dimeth-oxyethane, toluene, xylenes, acetonitrile (ACN), dimethysulfoxide, dimethylformamide (DMF), dimethylacetamide (DMA), N-methylpyrrolidone, and the like; preferably THF; at a temperature ranging from −70 to 100° C., preferably −65 to 40° C.; for a period of 2 h to 24 h. A compound of formula (VI) is protected employing established methodologies, such as those described in T. W. Greene and P. G. M. Wuts, “Protective Groups in Organic Synthesis,” 3 ed., John Wiley & Sons, 1999, to provide a mixture of compounds of formula (VIIa) and formula (VIIb), where R² is H or C₁₋₆alkyl, and PG is BOC.

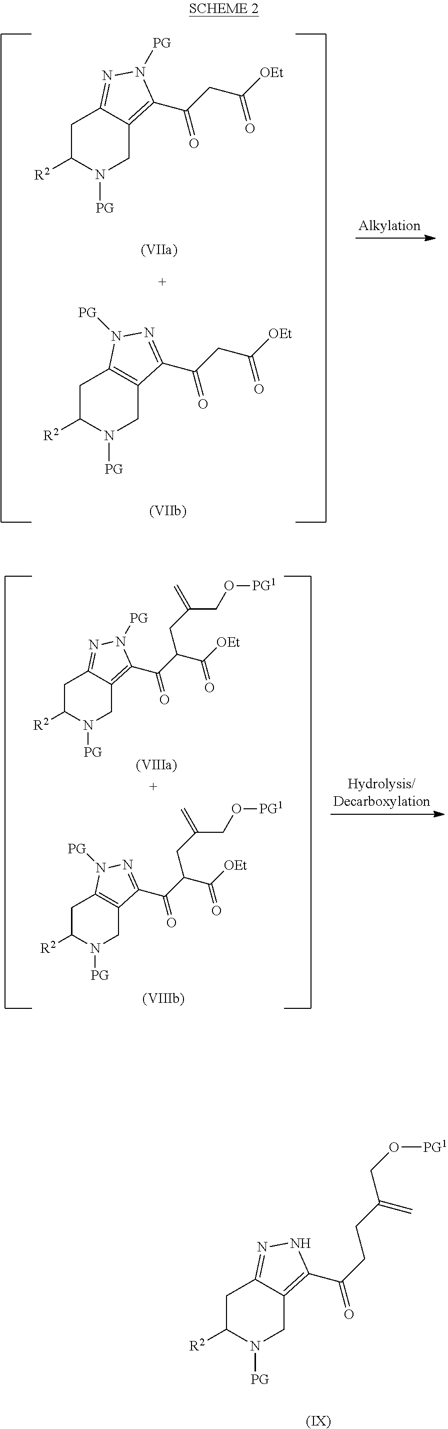

According to SCHEME 2, alkylation of 0-ketoester compounds of formula (VIIa) and (VIIb), where R² is H or C₁₋₆alkyl, and PG is BOC, is achieved employing an alkyl halide such as ((2-(bromomethyl)allyl)oxy)(tert-butyl)diphenylsilane, a base such as K₂CO₃; NaI; in a suitable solvent such as acetone, and the like; to provide a mixture of compounds of formulas (VIIIa) and (VIIIb). Hydrolysis/decarboxylation of a mixture of compounds of formula (VIIIa) and (VIIIb) is achieved using a base such as with potassium hydroxide, and the like; in a suitable solvent such a as MeOH, H₂O, or a mixture thereof to provide a compound of formula (IX).

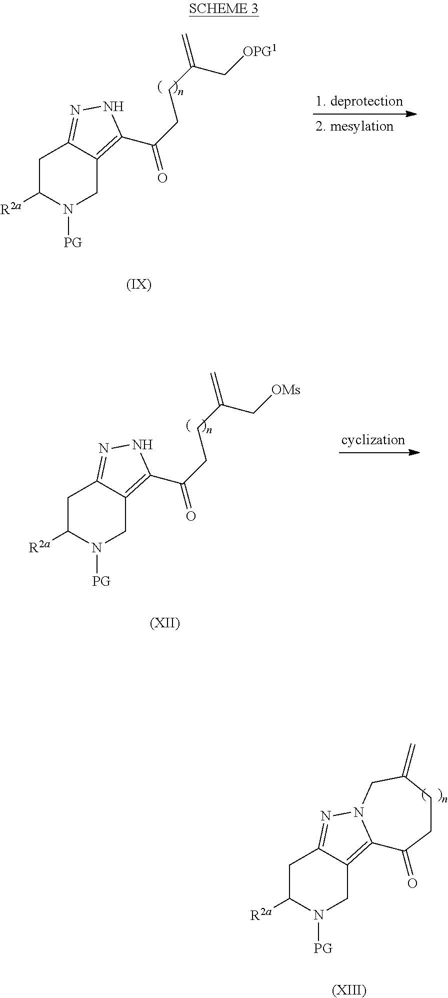

According to SCHEME 3, a compound of formula (IX), where R² is H or C₁₋₆alkyl, PG is BOC, PG¹ is TBDSP; is de-silylated with tetra-n-butylammonium fluoride (TBAF), in a suitable solvent such as THE and the like. Subsequent mesylation of the hydroxy employing methanesulfonyl chloride (mesyl chloride), a suitable base such as triethylamine (TEA), in a suitable solvent such as DCM, and the like, provides a compound of formula (XII). Intramolecular cyclization employing a base such as DBU, in a suitable solvent such as THF, and the like, provides compounds of formula (XIII), where n is 1. Compounds of formula (XIII), where n is 0 or 2 may be prepared in a manner analogous to compounds of formula (XIII) where n is 1.

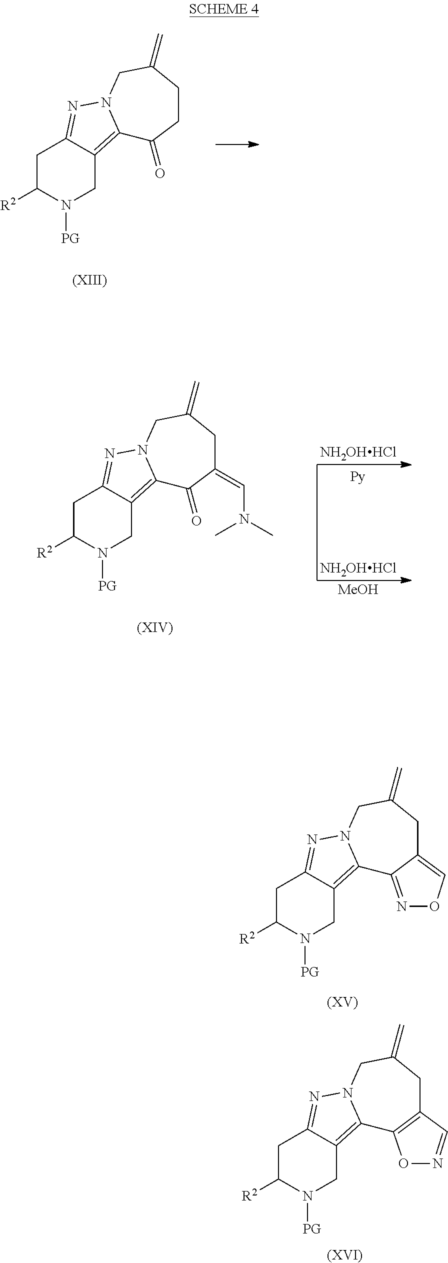

According to SCHEME 4, a compound of formula (XIII) is treated with DMA to afford the dimethyl enamine compound of formula (XIV), which upon treatment with hydroxylamine hydrochloride; in the presence of a tertiary base such as pyridine, and the like, at a temperature of about 70-115° C.; affords a compound of formula (XV). In a similar fashion, a compound of formula (XIV) is treated with hydroxylamine hydrochloride, in the presence of methanol, to afford a compound of formula (XVI).

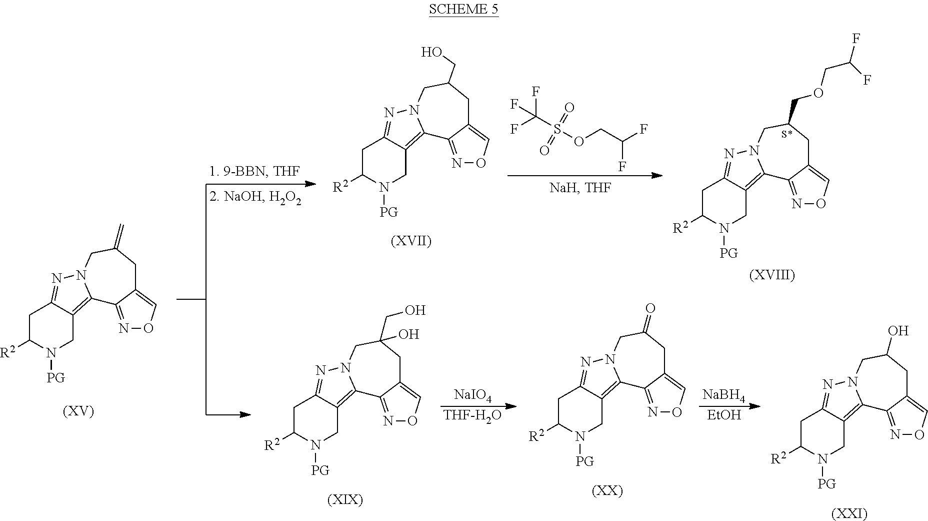

According to SCHEME 5, the alkenyl moiety of a compound of formula (XV) is regioselectively converted to its corresponding terminal alcohol compound of formula (XVII) by the action of 9-borabicyclo[3.3.1]nonane (9-BBN), followed by treatment with hydrogen peroxide, and hydroxide, to afford a compound of formula (XVII). Said terminal alcohol is further derivatized using methods well known to one of skill in the art. For example, the alcohol is oxidized to the corresponding aldehyde by the action of a suitable oxidizing agent such as manganese oxide. Alternatively, the alcohol functional group may also be alkylated with a suitable electrophile such as 2, 2-difluoroethyl trifluoromethanesulfonate; a suitable base such as NaH, and the like; in a suitable solvent such as THF, and the like; to provide a compound of formula (XVIII).

Alternatively, a compound of formula (XV), where R⁴ is H or C₁₋₄alkyl, undergoes an osmium-catalyzed dihydroxylation, employing conditions known to one skilled in the art, to provide a compound of formula (XIX). For example, a compound of formula (XV), where R⁴ is H or C₁₋₄alkyl; is reacted with an oxidant such as an osmium-containing compound like OsO₄ (or OsO₄ can also be prepared in situ by the oxidation of K₂OsO₂(OH)₄ with NMO); an amine oxide co-oxidant such as NMO, and the like; in a suitable solvent such as THF, acetone, H₂O, or a mixture thereof, to provide a compound of formula (XIX). A compound of formula (XIX) upon treatment with an oxidizing agent such as sodium periodate and the like; affords a compound of formula (XX). Reduction of the ketone of formula (XX) to an alcohol of formula (XXI) is achieved by reaction of a hydride source such as sodium borohydride; and the like, a suitable solvent such as an alcoholic solvent.

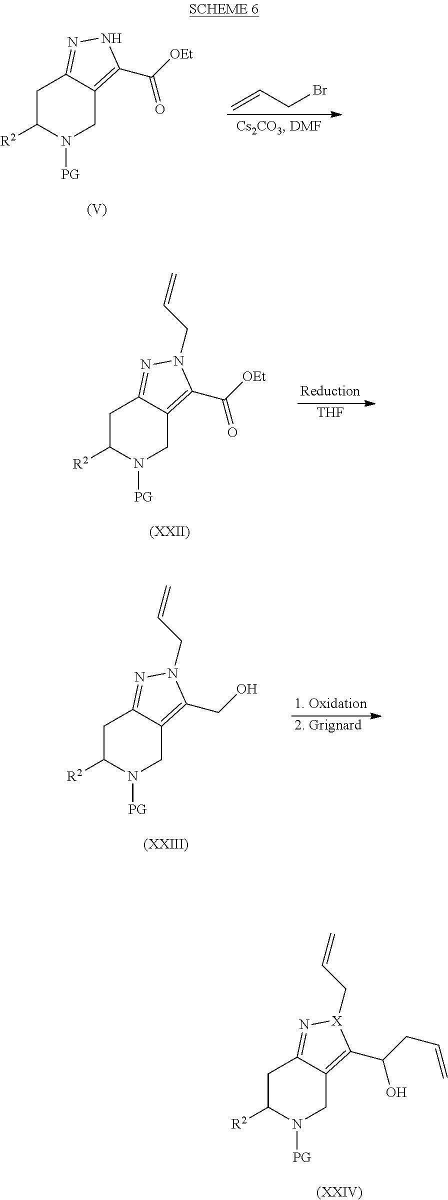

According to SCHEME 6, a commercially available or synthetically accessible alkyl halide, such as 3-bromoprop-1-ene, is reacted with a compound of formula (V), where R² is H or C₁₋₆alkyl; an inorganic base such as Cs₂CO₃, potassium carbonate, and the like; in a suitable solvent such as DMF, THF, pyridine, and the like; to provide a compound of formula (XXII). The ester functionality of a compound of formula (XXII) is reduced by a hydride source such as lithium aluminum hydride, sodium borohydride, or the like; in a suitable solvent such as THF, and the like; at temperatures ranging from −40° C. to 40° C.; to afford an alcohol of formula (XXIII). A compound of formula (XXIV) is prepared in two steps. In a first step, oxidation to the corresponding aldehyde is achieved employing conditions known to one skilled in the art, for example, Swern oxidation conditions ((COCl)₂/DMSO), or TPAP-NMO conditions. In a second step, reaction of the aldehyde intermediate with a Grignard reagent, such as allyl magnesium bromide; in an aprotic solvent, such as THF, and the like; at a temperature ranging from −40° C. to 40° C.; provides a compound of formula (XXIV), where PG is Boc, X is N, and R² is H or C₁₋₆alkyl.

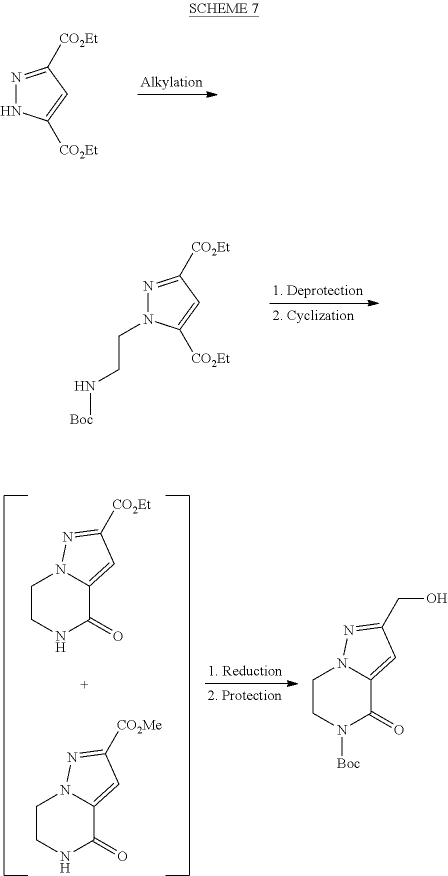

According to SCHEME 7, commercially available or synthetically accessible diethyl 1H-pyrazole-3,5-dicarboxylate is alkylated with tert-butyl N-(2-bromoethyl)carbamate; a base such as Cs₂CO₃, and the like; in a suitable solvent such as DMF, and the like; to provide diethyl 1-(2-((tert-butoxycarbonyl)amino)ethyl)-1H-pyrazole-3,5-dicarboxylate. Diethyl 1-(2-((tert-butoxycarbonyl)amino)ethyl)-1H-pyrazole-3,5-dicarboxylate is deprotected employing established methodologies, such as those described in T. W. Greene and P. G. M. Wuts, “Protective Groups in Organic Synthesis,” 3 ed., John Wiley & Sons, 1999; then subsequently treated under basic conditions to form a mixture of ethyl 4-oxo-4,5,6,7-tetrahydropyrazolo[1,5-a]pyrazine-2-carboxylate and methyl 4-oxo-4,5,6,7-tetrahydropyrazolo[1,5-a]pyrazine-2-carboxylate. A mixture of ethyl 4-oxo-4,5,6,7-tetrahydropyrazolo[1,5-a]pyrazine-2-carboxylate and methyl 4-oxo-4,5,6,7-tetrahydropyrazolo[1,5-a]pyrazine-2-carboxylate is with a hydride source such as LAH, and the like; followed by protection of the amino functionality using conventional methods, such as by treatment with Boc-anhydride, to afford tert-butyl 2-(hydroxymethyl)-6,7-dihydropyrazolo[1,5-a]pyrazine-5(4H)-carboxylate.

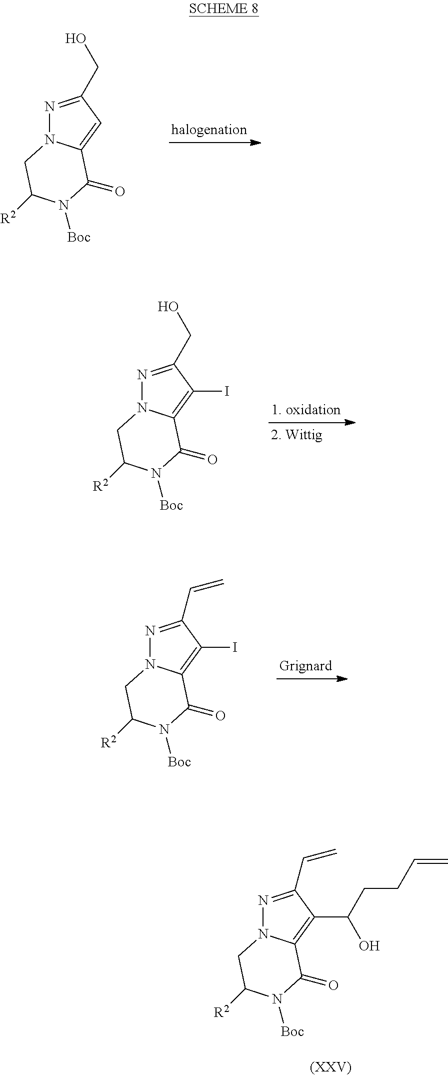

According to SCHEME 8, iodination of tert-butyl 2-(hydroxymethyl)-6,7-dihydropyrazolo[1,5-a]pyrazine-5(4H)-carboxylate is achieved employing a halogenating agent such as N-iodosuccinimide, and the like; in a suitable solvent such as ACN, and the like; at temperatures of about 15° C.; provides tert-butyl 2-(hydroxymethyl)-3-iodo-6,7-dihydropyrazolo[1,5-a]pyrazine-5(4H)-carboxylate. Subsequent oxidation of tert-butyl 2-(hydroxymethyl)-3-iodo-6,7-dihydropyrazolo[1,5-a]pyrazine-5(4H)-carboxylate is achieved with a suitable oxidizing agent, such as Dess-Martin periodinane (DMP); in a suitable solvent such as dichloromethane, and the like; at temperatures ranging from about 0° C. to about 25° C.; for a period of approximately 0.5 to 4 hours; to provide tert-butyl 2-formyl-3-iodo-6,7-dihydropyrazolo[1,5-a]pyrazine-5(4H)-carboxylate.

tert-Butyl 2-formyl-3-iodo-6,7-dihydropyrazolo[1,5-a]pyrazine-5(4H)-carboxylate is reacted with a Wittig type reagent such as methyltriphenylphosphonium bromide; a base such as NaHMDS, and the like; in an organic solvent such as THF, toluene, and the like; to provide tert-butyl 3-iodo-2-vinyl-6,7-dihydropyrazolo[1,5-a]pyrazine-5(4H)-carboxylate. tert-Butyl 3-iodo-2-vinyl-6,7-dihydropyrazolo[1,5-a]pyrazine-5(4H)-carboxylate is reacted under conventional Grignard reaction conditions with pent-4-enal; in the presence of an organomagnesium halide such as i-PrMgCl, and the like; in a suitable solvent such as THF, and the like; to provide a compound of formula (XXV), where PG is BOC, Y is N, and R² is H.

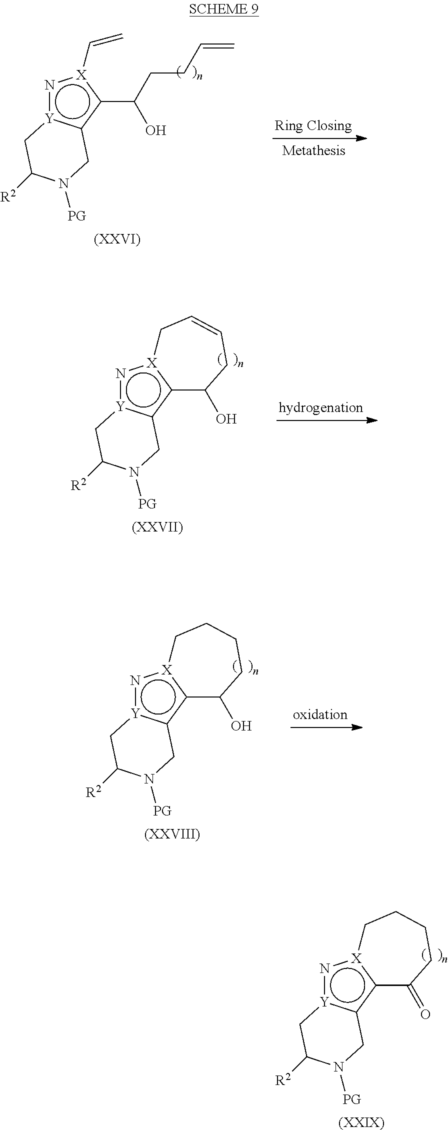

According to SCHEME 9, a compound of formula (XXVI), which includes compounds of formula (XXIV) and (XXV), undergoes a ring closing metathesis reaction employing dichloro[1,3-bis(2,4,6-trimethylphenyl)-2-imidazolidinylidene](2-isopropoxyphenylmethylene)ruthenium(II) (Hoveyda-Grubbs II catalyst); in a solvent such as DCM, and the like; for a period of 16-24 h; to provide a compound of formula (XXVII).

A compound of formula (XXVII), where PG is Boc, Y is C and X is N, and R² is H or C₁₋₆alkyl; is reduced employing hydrogenation conditions, in the presence of a palladium catalyst, including but not limited to, Pd on carbon, Pd(dppf)Cl₂ or Pd(PPh₃)₄; in a suitable solvent or solvent system such as DMF, methanol, dioxane/water, and the like; to provide a compound of formula (XXVIII), where PG is Boc, Y is C and X is N, n is 1, and R² is H or C₁₋₆alkyl.

Oxidation of a compound of formula (XXVIII) to a compound of formula (XXIX) is achieved employing conditions known to one skilled in the art. For example, reaction of an alcohol compound of formula (XXVIII), with the oxidation catalyst tetrapropylammonium perruthenate (TPAP); and N-methylmorpholine N-oxide (NMO) as the co-oxidant; in a suitable solvent such as ACN, DCM, DMF, and the like; provides a compound of formula (XXIX), where X is N and Y is C.

In a similar fashion, a compound of formula (XXVII), where X is C and Y is N; is first oxidized under TPAP conditions previously described, followed by reduction of the double bond employing hydrogenation conditions previously described to provide a compound of formula (XXIX), where PG is Boc, Y is N and X is C, n is 1, and R² is H or C₁₋₆alkyl.

Compounds of formula (XXIX), where n is 0 or 2 may be prepared in a manner analogous to compounds of formula (XXIX) where n is 1.

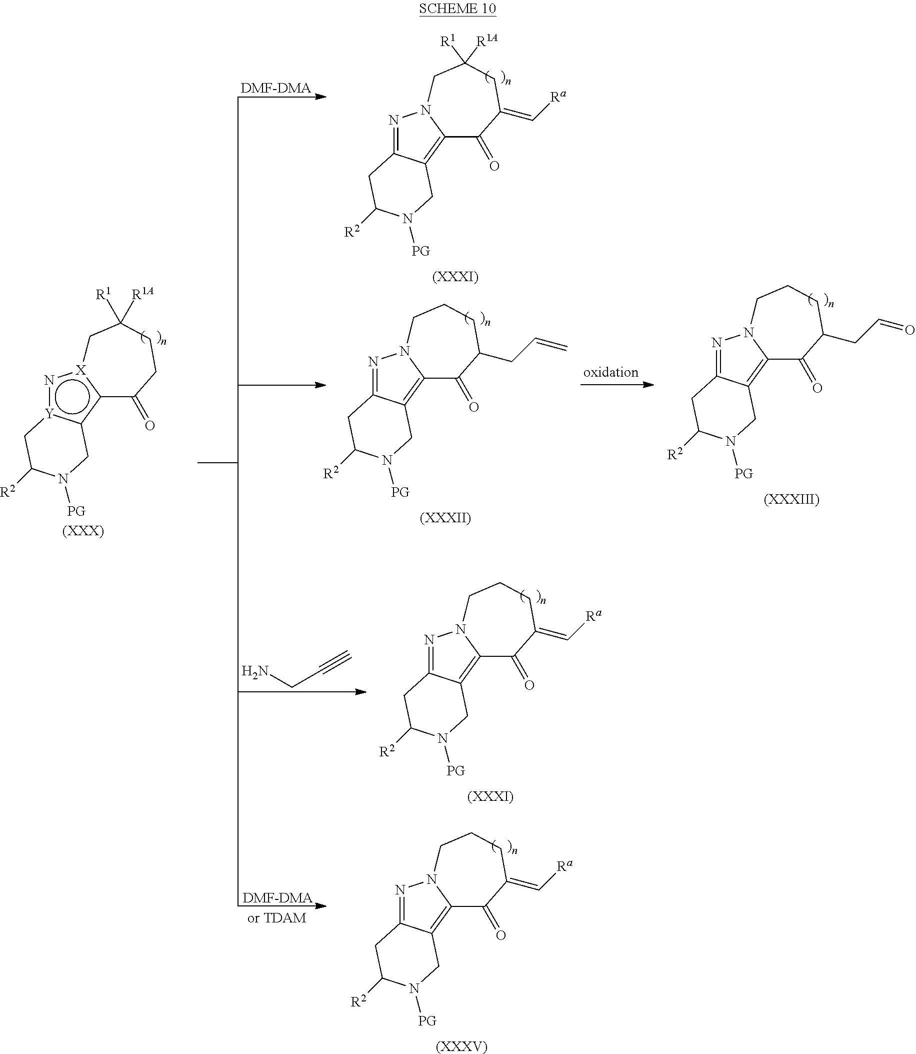

According to SCHEME 10, a ketone compound of formula (XXX), where X is N, Y is C, R¹ and R^{1A} are H, or R¹ and R^{1A} come together to form a methylene, R² is H or C₁₋₆alkyl, and PG is Boc; is condensed with dimethylformamide-dimethyl acetal (DMF-DMA) to afford a compound of formula (XXXI) where R^{a} is OH or N(CH₃)₂, and n is 1.

A compound of formula (XXX), where X is N, Y is C, R¹ and R^{1A} are H, R² is H or C₁₋₆alkyl, n is 1, and PG is BOC; is alkylated with allyl bromide; in the presence of a strong organometallic base such as LDA; in the presence of HMPA; in an aprotic organic solvent such as THF, and the like; to afford a compound of formula (XXXII). Oxidation of a compound of formula (XXXII) to an aldehyde compound of formula (XXXIII) is achieved under conditions known to one skilled in the art, for example, osmium tetroxide, sodium periodate, Swern oxidation conditions, and the like.

A compound of formula (XXX), where X is N, Y is C, R¹ and R^{1A} are H, R² is H or C₁₋₆alkyl, n is 1, and PG is BOC; is reacted under amination/cyclization conditions with propargyl amine; a gold catalyst such as NaAuCll₄.2H₂O, and the like; in a suitable solvent such as EtOH, and the like; to provide a compound of formula (XXXIV).

A ketone compound of formula (XXX), where X is C, Y is N, R¹ and R^{1A} are H, R² is H or C₁₋₆alkyl, and PG is Boc; is condensed with dimethylformamide-dimethyl acetal (DMF-DMA) to afford an enaminone compound of formula (XXXV). In an alternate method, tris(dimethylamino)methane (TDAM) is reacted with a compound of formula (XXX), in a solvent such as toluene, and the like; at temperatures of about 115° C.; for a period of 12-20 h; to provide a compound of formula (XXXV), where R^{a} is N(CH₃)₂, and n is 1.

Compounds of formulas (XXXI), (XXXIII), (XXXIV), and (XXXV), where n is 0 or 2 may be prepared in a manner analogous to compounds of formulas (XXXI), (XXXIII), (XXXIV), and (XXXV), where n is 1.

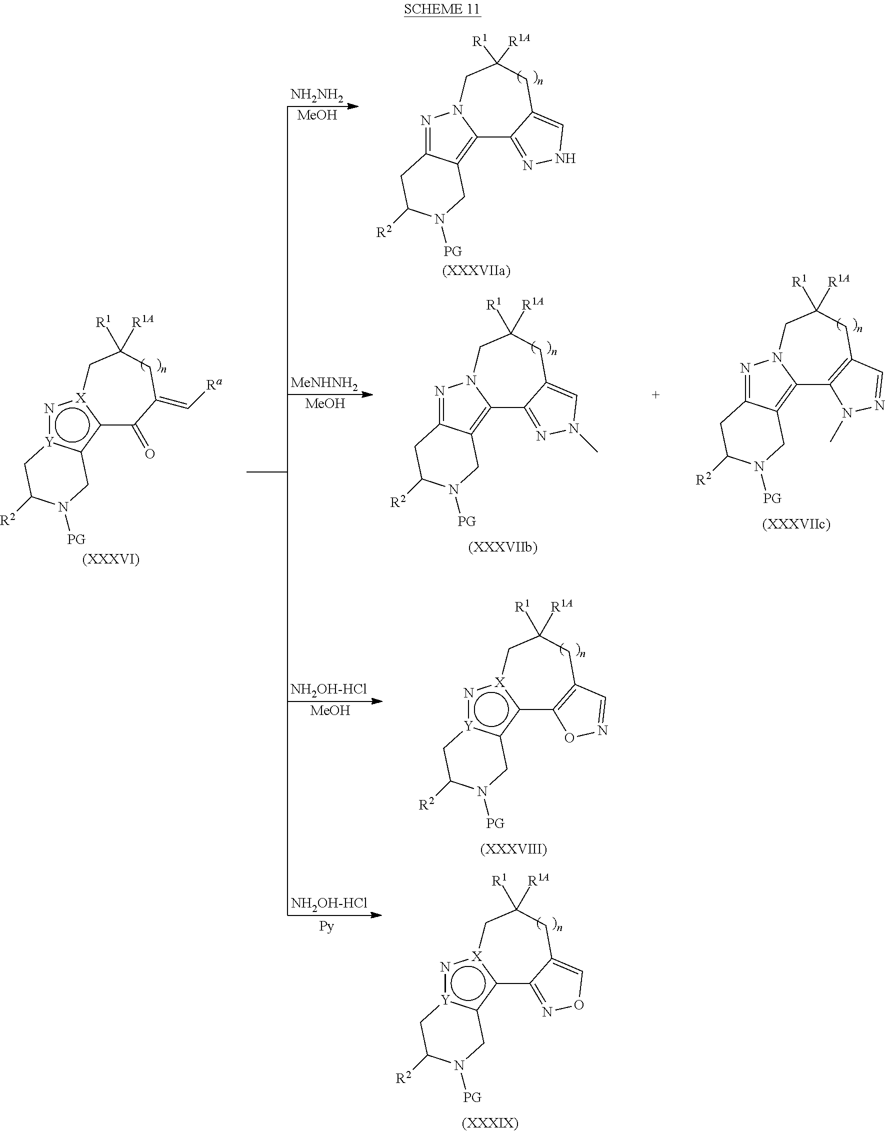

According to SCHEME 11, compounds of formulas (XXXVIIa, XXXVIIb, XXXVIIc), are prepared by reacting a compound of formula (XXXVI), where X is N, Y is C, n is 1, and R^{a} is OH; with a hydrazine such as methylhydrazine or hydrazine hydrate; in a suitable solvent such as MeOH, and the like.

A compound of formula (XXXVI), where R^{a} is OH or N(CH₃)₂; is treated with hydroxylamine hydrochloride; in the presence of a tertiary base such as pyridine, and the like, at temperatures ranging from 70° C. to 115° C.; to afford an isoxazole compound of formula (XXXVIII). In a similar fashion, a compound of formula (XXXVI) is treated with hydroxylamine hydrochloride, in a suitable solvent such as MeOH and the like, at a temperature of about 70° C., to provide an isoxazole compound of formula (XXXIX), where n is 1.

Compounds of formulas (XXXVIIa), (XXXVIIb), (XXXVIIc), (XXXVIII), and (XXXIX) where n is 0 or 2 may be prepared in a manner analogous to compounds of formulas (XXXVIIa), (XXXVIIb), (XXXVIIc), (XXXVIII), and (XXXIX), where n is 1.

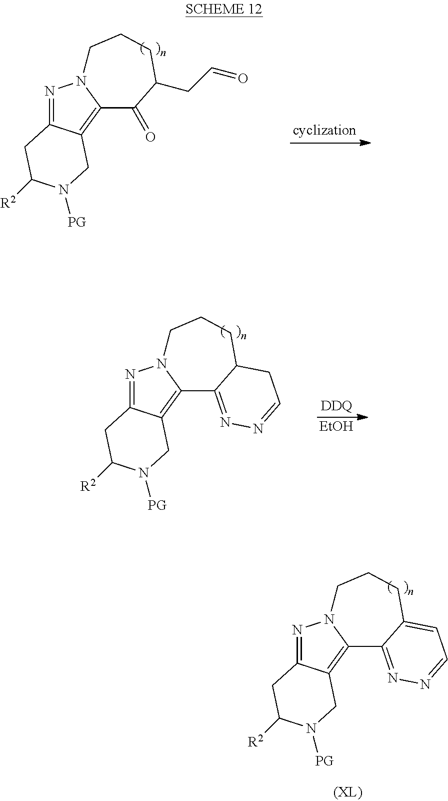

According to SCHEME 12, tert-butyl 11-oxo-10-(2-oxoethyl)-3,4,8,9,10,11-hexahydro-1H-pyrido[4′,3′:3,4]pyrazolo[1,5-a]azepine-2(7H)-carboxylate is treated with hydrazine hydrate to afford tert-butyl 4a,5,6,7,10,11-hexahydro-4H-pyridazino[3,4-c]pyrido[4′,3′:3,4]pyrazolo[1,5-a]azepine-12(13H)-carboxylate. tert-butyl 4a,5,6,7,10,11-hexahydro-4H-pyridazino[3,4-c]pyrido[4′,3′:3,4]pyrazolo[1,5-a]azepine-12(13H)-carboxylate is oxidized with a reagent such as DDQ, and the like; in a suitable solvent such as THF; at a temperature of about 0° C.; affords the aromatized compound of formula (XL), where n is 1, R² is H, and PG is Boc.

A compound of formula (XL), where n is 0 or 2, and R² is H or C₁₋₆alkyl, may be prepared in a manner analogous to a compound of formula (XL), where n is 1.

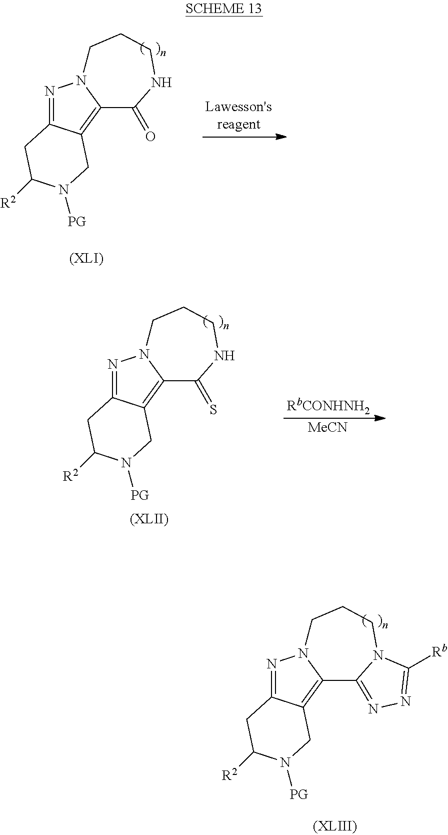

According to SCHEME 13, a compound of formula (XLI) is converted to the thioamide compound of formula (XLII), employing Lawesson's reagent. For example, tert-butyl 11-oxo-3,4,8,9,10,11-hexahydro-1H-pyrido[4′,3′:3,4]pyrazolo[1,5-a][1,4]diazepine-2(7H)-carboxylate (as described in PCT Int. Appl. WO2018005883, Jan. 4, 2018) is treated with Lawesson's reagent; in a suitable solvent such as toluene, and the like; at a temperature of about 110° C.; to provide tert-butyl 11-thioxo-3,4,8,9,10,11-hexahydro-1H-pyrido[4′,3′:3,4]pyrazolo[1,5-a][1,4]diazepine-2(7H)-carboxylate. A compound of formula (XLII), is cyclized to form a compound of formula (XLIII). For example, tert-butyl 11-thioxo-3,4,8,9,10,11-hexahydro-1H-pyrido[4′,3′:3,4]pyrazolo[1,5-a][1,4]diazepine-2(7H)-carboxylate is cyclized with an R^{b}-substituted hydrazide (wherein R^{b} is hydrogen or CH₃: Hg(OAc)₂; in a suitable solvent such as ACN, and the like; to afford a compound of formula (XLIII), where R² is H or C₁₋₆alkyl, PG is Boc, n is 1, and R^{b} is H or CH₃.

A compound of formula (XLIII), where n is 0 or 2, and R² is H or C₁₋₆alkyl, may be prepared in a manner analogous to a compound of formula (XLIII), where n is 1.

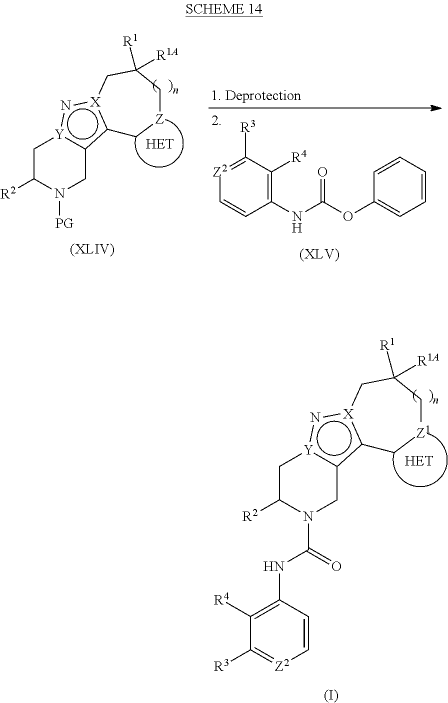

According to SCHEME 14, a compound of formula (XLIV) (which encompasses compounds of formulas (XV), (XVI), (XVIII), (XXI), (XXXIV), (XXXVIIa), (XXXVIIb), XXXVIIc), (XXXVIII), (XXXIX), (XL), and (XLIII)), is deprotected employing conditions known to one skilled in the art. Subsequent reaction with a commercially available or synthetically accessible compound of formula (XLV), where Z², R³, and R⁴ are as defined in claim 1; a suitable base such as TEA, and the like; in a suitable solvent such as DCM, and the like; provides a compound of Formula (I).

Compounds of Formula (I) may be converted to their corresponding salts using methods known to one of ordinary skill in the art. For example, an amine of Formula (I) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) may be obtained in crystalline form by recrystallization from polar solvents (including mixtures of polar solvents and aqueous mixtures of polar solvents) or from non-polar solvents (including mixtures of non-polar solvents).

Where the compounds according to this present disclosure have at least one chiral center, they may accordingly exist as enantiomers. Where the compounds possess two or more chiral centers, they may additionally exist as diastereomers. It is to be understood that all such isomers and mixtures thereof are encompassed within the scope of the present disclosure.

Compounds of formulas represented in the SCHEMES above represented as “stereometric mixture” (means a mixture of two or more stereoisomers and includes enantiomers, diastereomers and combinations thereof) are separated by SFC resolution.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

### General Procedures

The following specific examples are provided to further illustrate the present disclosure and various preferred embodiments.

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

Preparative reverse-phase high performance liquid chromatography (RP HPLC) was performed on either:

METHOD A. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (with 0.225% FA) over 10 min and then hold at 100% ACN for 2 min, at a flow rate of 25 mL/min. or
METHOD B. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.1% TFA) over 10 min and then hold at 100% ACN for 2 min, at a flow rate of 25 mL/min. or
METHOD C. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.05% HCl) over 10 min and then hold at 100% ACN for 2 min, at a flow rate of 25 mL/min. or
METHOD D. A Gilson GX-281 semi-prep-HPLC with Phenomenex Gemini C18 (10 μm, 150×25 mm), AD (10 μm, 250 mm×30 mm), or Waters XBridge C18 column (5 μm, 150×30 mm), mobile phase of 0-99% ACN in water (with 0.05% ammonia hydroxide v/v) over 10 min and then hold at 100% ACN for 2 min, at a flow rate of 25 mL/min. or
METHOD E. A Gilson GX-281 semi-prep-HPLC with Phenomenex Gemini C18 (10 μm, 150×25 mm), or Waters XBridge C18 column (5 μm, 150×30 mm), mobile phase of 5-99% ACN in water (10 mM NH4HCO3) over 10 min and then hold at 100% ACN for 2 min, at a flow rate of 25 mL/min.
Preparative supercritical fluid high performance liquid chromatography (SFC) was performed either on a Thar 80 Prep-SFC system, or Waters 80Q Prep-SFC system from Waters. The ABPR was set to 100 bar to keep the CO₂ in SF conditions, and the flow rate may verify according to the compound characteristics, with a flow rate ranging from 50 g/min to 70 g/min. The column temperature was ambient temperature

Mass spectra (MS) were obtained on a SHIMADZU LCMS-2020 MSD or Agilent 1200\G6110A MSD using electrospray ionization (ESI) in positive mode unless otherwise indicated. Calculated (calcd.) mass corresponds to the exact mass.

Nuclear magnetic resonance (NMR) spectra were obtained on Bruker model AVIII 400 spectrometers. Definitions for multiplicity are as follows: s=singlet, d=doublet, t=triplet, q=quartet, m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 12.0, ChemDraw Ultra 14.0 (CambridgeSoft Corp., Cambridge, Mass.) or ACD/Name Version 10.01 (Advanced Chemistry).

Compounds designated as R* or S* are enantiopure compounds where the absolute configuration was not determined.

### Intermediate 1: tert-Butyl 5-methylene-5,6,9,10-tetrahydro-4H-isoxazolo[3,4-c]pyrido[4′,3′:3,4]pyrazolo[1,5-a]azepine-11(12H)-carboxylate

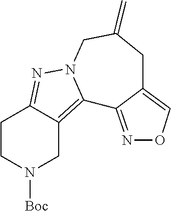 [SMILES: C=C1Cc2conc2-c2c3c(nn2C1)CCN(C(=O)OC(C)(C)C)C3]

Step A. tert-Butyl 3-(3-ethoxy-3-oxopropanoyl)-6,7-dihydro-2H-pyrazolo[4,3-c]pyridine-(4H)-carboxylate. To a solution of ethyl acetate (20.88 g, 237.02 mmol, 23.20 mL) in THE (120 mL) was added NaHMDS (1 M, 474.04 mL) at −65° C. under N₂. A solution of 5-tert-butyl 3-ethyl 6,7-dihydro-2H-pyrazolo[4,3-c]pyridine-3,5(4H)-dicarboxylate (preparation as described in WO2018005881, publication date Jan. 4, 2018) (28 g, 94.81 mmol) in THE (200 mL) was added dropwise into the mixture over 1 h at −65° C. The mixture was stirred at 45° C. for 10 h. The mixture was quenched with HCl (1 M aq, 1500 mL) and diluted with ethyl acetate (1500 mL). The organic phases were separated and dried over Na₂SO₄, filtered and concentrated in vacuo. The residue was purified by column chromatography (SiO₂, petroleum ether/ethyl acetate=10/1 to 1/1) to give the title compound (28.4 g, 84.18 mmol, 88.79% yield, 100% purity) as a yellow solid. MS (ESI): mass calcd. for C₁₆H₂₃N₃O₅, 337.16; m/z found, 360.1 [M+Na]⁺.

### Step B. Mixture of di-tert-butyl 3-(3-ethoxy-3-oxopropanoyl)-6,7-dihydro-2H-pyrazolo[4,3-c]pyridine-2,5(4H)-dicarboxylate and di-tert-butyl 3-(3-ethoxy-3-oxopropanoyl)-6,7-dihydro-1H-pyrazolo[4,3-c]pyridine-1,5(4H)-dicarboxylate

To a solution of tert-butyl 3-(3-ethoxy-3-oxopropanoyl)-6,7-dihydro-2H-pyrazolo[4,3-c]pyridine-5(4H)-carboxylate (18 g, 53.35 mmol), TEA (16.20 g, 160.06 mmol, 22.28 mL) and DMAP (651.82 mg, 5.34 mmol) in DCM (200 mL) was added Boc₂O (11.64 g, 53.35 mmol, 12.26 mL), the mixture was stirred at 15° C. for 2 h. The mixture was poured into 1 M HCl aq (250 mL) and extracted with ethyl acetate (200 mL×2). The combined organic phases were washed with brine (200 mL), dried with anhydrous Na₂SO₄, filtered and the filtrate concentrated under reduced pressure. The residue was purified by silica flash column chromatography (eluent of 0˜20% ethyl acetate/petroleum ether) to give the title compound (20 g, 22.86 mmol, 42.84% yield, 100% purity) as a colorless oil. MS (ESI): mass calcd. for C₂₁H₃₁N₃O₇, 437.22; m/z found, 460.1 [M+Na]⁺/897.2 [2M+23]⁺.

Step C. Mixture of di-tert-butyl 3-(4-(((tert-butyldiphenylsilyl)oxy)methyl)-2-(ethoxy-carbonyl)pent-4-enoyl)-6,7-dihydro-2H-pyrazolo[4,3-c]pyridine-2,5(4H)-dicarboxylate and di-tert-butyl3-(4-(((tert-butyldiphenylsilyl)oxy)methyl)-2-(ethoxycarbonyl)pent-4-enoyl)-6,7-dihydro-1H-pyrazolo[4,3-c]pyridine-1,5(4H)-dicarboxylate. To a mixture of di-tert-butyl 3-(3-ethoxy-3-oxopropanoyl)-6,7-dihydro-2H-pyrazolo[4,3-c]pyridine-2,5(4H)-dicarboxylate and di-tert-butyl 3-(3-ethoxy-3-oxopropanoyl)-6,7-dihydro-1H-pyrazolo[4,3-c]pyridine-1,5(4H)-dicarboxylate (14.00 g, 32.04 mmol) in acetone (150 mL) was added K₂CO₃ (6.64 g, 48.05 mmol), NaI (960.39 mg, 6.41 mmol) and 2-(bromomethyl)allyloxy-tert-butyl-diphenylsilane (14.97 g, 38.44 mmol). The mixture was stirred at 55° C. for 4 h. The mixture was poured into HCl (400 mL, 1 M aq) at 0° C. and extracted with ethyl acetate (300 mL×3). The combined organic phases were washed with brine (500 mL), dried with anhydrous Na₂SO₄, filtered, and the filtrate concentrated under reduced pressure. The residue was purified by column chromatography (SiO₂, petroleum ether/ethyl acetate=30/1 to 20/1) to afford the title compound (13.5 g, 16.83 mmol, 52.53% yield, 93% purity) (TLC, petroleum ether/ethyl acetate=3/1) as a yellow oil. MS (ESI): mass calcd. for C₄₁H₅₅N₃O₃Si, 745.38; m/z found, 768.5 [M+Na]⁺.

Step D. tert-Butyl 3-(4-(((tert-butyldiphenylsilyl)oxy)methyl)pent-4-enoyl)-6,7-dihydro-2H-pyrazolo[4,3-c]pyridine-5(4H)-carboxylate. To a mixture of di-tert-butyl 3-(4-(((tert-butyl-diphenylsilyl)oxy)methyl)-2-(ethoxycarbonyl)pent-4-enoyl)-6,7-dihydro-2H-pyrazolo[4,3-c]pyridine-2,5(4H)-dicarboxylate and di-tert-butyl 3-(4-(((tert-butyldiphenylsilyl)oxy)-methyl)-2-(ethoxycarbonyl)pent-4-enoyl)-6,7-dihydro-1H-pyrazolo[4,3-c]pyridine-1,5(4H)-dicarboxylate (13.5 g, 16.83 mmol) in MeOH (50 mL) was added a solution of KOH (1.89 g, 33.66 mmol) in water (10 mL). The mixture was stirred at 65° C. for 3 h. The mixture was poured into HCl (1M, aq, 300 mL) and extracted with ethyl acetate (200 mL×3). The combined organic phases were washed with brine (200 mL), dried with anhydrous Na₂SO₄, filtered, and the filtrate concentrated under reduced pressure. The residue was purified by silica gel chromatography (SiO₂:petroleum ether/ethyl acetate=3/1) to afford the title compound (8.9 g, 15.51 mmol, 92.15% yield) as a yellow oil. MS (ESI): mass calcd. for C₃₃H₄₃N₃O₄Si, 573.3; m/z found, 574.4 [M+H]⁺.

Step E. tert-Butyl 3-(4-(hydroxymethyl)pent-4-enoyl)-6,7-dihydro-2H-pyrazolo [4,3-c]pyridine-5(4H)-carboxylate. To a solution of tert-butyl 3-(4-(((tert-butyldiphenylsilyl)-oxy)methyl)pent-4-enoyl)-6,7-dihydro-2H-pyrazolo[4,3-c]pyridine-5(4H)-carboxylate (14 g, 21.96 mmol) in THE (50 mL) was added TBAF (1 M, 32.94 mL). The mixture was stirred at 30° C. for 12 h. The mixture was poured into water (100 mL) and extracted with ethyl acetate (80 mL×3). The combined organic phases were washed with brine (100 mL), dried with anhydrous Na₂SO₄, filtered and the filtrate concentrated under reduced pressure. The residue was purified by silica gel chromatography (petroleum ether/ethyl acetate=2/1 to 1/1) to afford the title compound (6.3 g, 18.41 mmol, 83.83% yield, 98% purity) as a white solid. MS (ESI)/mass calcd. for C₁₇H₂₅N₃O₄, 335.2; m/z found, 358.1 [M+Na]⁺; ¹H NMR (400 MHz, CDCl₃) δ=5.05 (s, 1H), 4.91 (s, 1H), 4.67 (s, 2H), 4.16 (s, 2H), 3.72 (t, J=5.4 Hz, 2H), 3.15 (s, 2H), 2.79 (t, J=5.6 Hz, 2H), 2.53 (t, J=7.2 Hz, 2H), 1.49 (s, 9H).

Step F. tert-Butyl 3-(4-(((methylsulfonyl)oxy)methyl)pent-4-enoyl)-6,7-dihydro-2H-pyrazolo[4,3-c]pyridine-5(4H)-carboxylate. To a solution of tert-butyl 3-(4-(hydroxymethyl)-pent-4-enoyl)-6,7-dihydro-2H-pyrazolo[4,3-c] pyridine-5(4H)-carboxylate (6.3 g, 18.41 mmol) and TEA (5.59 g, 55.23 mmol, 7.69 mL) in DCM (30 mL) was added MsCl (4.73 g, 41.29 mmol, 3.20 mL) at 0° C. under N₂. The mixture was stirred at 0° C. for 1 h. The mixture was poured into water (60 mL) and extracted with ethyl acetate (60 mL×3). The combined organic phases were washed with brine (60 mL), dried with anhydrous Na₂SO₄, filtered, and the filtrate concentrated under reduced pressure to afford the title compound (8.2 g, crude) as a yellow oil. MS (ESI): mass calcd. for C₁₈H₂₇N₃O₆S, 413.2; m/z found, 414.1 [M+H]⁺.

Step G. tert-Butyl 8-methylene-11-oxo-3,4,8,9,10,11-hexahydro-1H-pyrido[4′,3′:3,4]-pyrazolo[1,5-a]azepine-2(7H)-carboxylate. To a solution of tert-butyl3-(4-(((methylsulfonyl)-oxy)methyl)pent-4-enoyl)-6,7-dihydro-2H-pyrazolo[4,3-c] pyridine-5(4H)-carboxylate (8.2 g, crude) in THE (60 mL) was added DBU (7.06 g, 46.37 mmol, 6.99 mL) at 30° C. under N₂. The mixture was stirred at 30° C. for 1 h. The mixture was poured into water (50 mL), extracted with ethyl acetate (50 mL×3). The combined organic phases were washed with brine (50 mL), dried over anhydrous Na₂SO₄, filtered, and the filtrate concentrated under reduced pressure. The residue was purified by silica gel chromatography (silica gel, petroleum ether/ethyl acetate=10/1-8/1) to afford the title compound (4.2 g, 11.25 mmol, 85% purity) as a colorless oil. MS (ESI): mass calcd. for C₁₇H₂₃N₃O₃, 317.2; m/z found, 318.2 [M+H]⁺; ¹H NMR (400 MHz, CDCl₃) δ=5.22 (s, 1H), 5.09 (s, 1H), 5.03 (s, 2H), 4.62 (s, 2H), 3.68 (s, 2H), 2.93-2.87 (m, 2H), 2.74 (s, 4H), 1.47 (s, 9H).

Step H. tert-Butyl 10-((dimethylamino)methylene)-8-methylene-11-oxo-3,4,8,9,10,11-hexahydro-1H-pyrido[4′,3′:3,4]pyrazolo[1,5-a]azepine-2(7H)-carboxylate. A solution of tert-butyl 8-methylene-11-oxo-3,4,8,9,10,11-hexahydro-1H-pyrido[4′,3′:3,4]pyrazolo[1,5-a]azepine-2(7H)-carboxylate (4.2 g, 11.25 mmol) in DMF-DMA (15 mL) was stirred at 80° C. for 12 h. The mixture was concentrated under reduced pressure. The residue was poured into water (30 mL) and extracted with ethyl acetate (20 mL×2). The combined organic phases were washed with brine (20 mL×2), dried over anhydrous Na₂SO₄, filtered, and the filtrate concentrated under reduced pressure to afford the title compound (4.5 g, crude) as a yellow solid. MS (ESI): mass calcd. for C₂₀H₂₈N₄O₃, 372.2; m/z found, 395.1 [M+Na]⁺.

Step I. tert-Butyl 5-methylene-5,6,9,10-tetrahydro-4H-isoxazolo[3,4-c]pyrido[4′,3′:3,4]-pyrazolo[1,5-a]azepine-11(12H)-carboxylate. To a solution of tert-butyl-10-((dimethyl-amino)-methylene)-8-methylene-11-oxo-3,4,8,9,10,11-hexahydro-1H-pyrido[4′,3′:3,4]-pyrazolo[1,5-a]azepine-2(7H)-carboxylate (4.5 g, crude) in Py (50 mL) was added NH₂OH.HCl (5.04 g, 72.53 mmol). The mixture was stirred at 115° C. for 12 h. The mixture was concentrated under reduced pressure. The residue was poured into HCl (1N, aq, 40 mL) and stirred for 1 min. The aqueous phase was extracted with ethyl acetate (40 mL×2). The combined organic phases were washed with brine (30 mL×2), dried over anhydrous Na₂SO₄, filtered, and the filtrate concentrated under reduced pressure. The residue was purified by column chromatography (SiO₂, petroleum ether/ethyl acetate=10/1 to 5/1) to afford the title compound (2.1 g, 5.95 mmol, 97% purity) as a white solid. MS (ESI): mass calcd. for C₁₈H₂₂N₄O₃, 342.2; m/z found, 343.1 [M+H]⁺. ¹H NMR (400 MHz, CDCl₃) δ=8.32 (s, 1H), 5.34 (s, 1H), 5.26 (s, 1H), 4.93 (s, 2H), 4.68 (s, 2H), 3.75 (s, 2H), 3.64 (s, 2H), 2.79 (s, 2H), 1.50-1.47 (m, 9H).

### Intermediate 2: tert-Butyl 5-(hydroxymethyl)-5,6,9,10-tetrahydro-4H-isoxazolo[3,4-c]pyrido-[4′,3′:3,4]pyrazolo[1,5-a]azepine-11(12H)-carboxylate

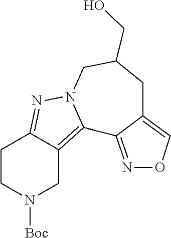 [SMILES: CC(C)(C)OC(=O)N1CCc2nn3c(c2C1)-c1nocc1CC(CO)C3]

To a solution of tert-butyl 5-methylene-5,6,9,10-tetrahydro-4H-isoxazolo[3,4-c]pyrido-[4′,3′:3,4]pyrazolo[1,5-a]azepine-11(12H)-carboxylate (Intermediate 1, product from Step I, 480 mg, 1.40 mmol) in THE (5 mL) was added 1,9-BBN (0.5 M, 56.08 mL) at −10° C. The mixture was stirred at −10° C. for 2 h then a solution of NaOH (560.72 mg, 14.02 mmol) in water (5 mL) was added at −30° C., followed by H₂O₂ (3.18 g, 28.04 mmol, 2.69 mL, 30% purity). The mixture was stirred at 15° C. for 16 h. The mixture was quenched with sat.aq NaHSO₃ (50 mL) and extracted with EtOAc (80 mL×3), the combined organic layers were dried over Na₂SO₄, filtered, and the filtrate concentrated under reduced pressure. The residue was purified by column chromatography (SiO₂, petroleum ether/ethyl acetate=50%˜100%) to afford the title compound (460 mg, 1.24 mmol, 88.31% yield, 97% purity) as a white solid. MS (ESI): mass calcd. for C₁₈H₂₄N₄O₄, 360.18; m/z found, 361.0 [M+H]⁺.

### Intermediate 3: (5S*)-tert-Butyl 5-((2,2-difluoroethoxy)methyl)-5,6,9,10-tetrahydro-4H-isoxazolo[3,4-c]pyrido[4′,3′:3,4]pyrazolo[1,5-a]azepine-11(12H)-carboxylate

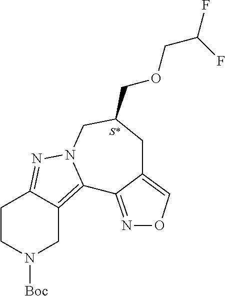 [SMILES: CC(C)(C)OC(=O)N1CCc2nn3c(c2C1)-c1nocc1C[C@H](COCC(F)F)C3]

Step A. (5S*)-tert-Butyl 5-(hydroxymethyl)-5,6,9,10-tetrahydro-4H-isoxazolo[3,4-c]pyrido-[4′,3′:3,4]pyrazolo[1,5-a]azepine-11(12H)-carboxylate and (5R*)-tert-Butyl-5-(hydroxy-methyl)-5,6,9,10-tetrahydro-4H-isoxazolo[3,4-c] pyrido[4′,3′:3,4]pyrazolo[1,5-a]azepine-11(12H)-carboxylate. tert-Butyl 5-(hydroxymethyl)-5,6,9,10-tetrahydro-4H-isoxazolo[3,4-c]pyrido[4′,3′:3,4]pyrazolo[1,5-a]azepine-11(12H)-carboxylate (Intermediate 2) was isolated by SFC (condition: column: IC (250 mm×30 mm, 10 um); mobile phase: [0.1% NH₃ H₂O IPA]; B %: 45%-45%, 6.1 min; 100 min) to give (5S*)-tert-butyl 5-(hydroxymethyl)-5,6,9,10-tetrahydro-4H-isoxazolo[3,4-c]pyrido [4′,3′:3,4]pyrazolo[1,5-a]azepine-11(12H)-carboxylate (Peak 1 on SFC (IC-3S_4_40_3ML Column: Chiralpak IC-3 100×4.6 mm I.D., 3 um Mobile phase: 40% iso-propanol (0.05% DEA) in CO₂ Flow rate: 3 mL/min Wavelength: 220 nm), retention time=1.369 min, 136 mg, 97% purity) as a white solid and (5R*)-tert-butyl 5-(hydroxymethyl)-5,6,9,10-tetrahydro-4H-isoxazolo[3,4-c]pyrido[4′,3′:3,4]pyrazolo[1,5-a]azepine-11(12H)-carboxylate (Peak 2 on SFC (IC-3S_4_40_3ML Column: Chiralpak IC-3 100×4.6 mm I.D., 3 um Mobile phase: 40% iso-propanol (0.05% DEA) in CO₂ Flow rate: 3 mL/min Wavelength: 220 nm), retention time=1.627 min, 82 mg, 97% purity) as a white solid.

Step B. (5S*)-tert-Butyl 5-((2,2-difluoroethoxy)methyl)-5,6,9,10-tetrahydro-4H-isoxazolo-[3,4-c]pyrido[4′,3′:3,4]pyrazolo[1,5-a]azepine-11(12H)-carboxylate. To a solution of (5S*)-tert-butyl 5-(hydroxymethyl)-5,6,9,10-tetrahydro-4H-isoxazolo[3,4-c]pyrido[4′,3′:3,4]-pyrazolo[1,5-a]azepine-11(12H)-carboxylate (135.00 mg, 363.34 umol) in THE (2 mL) was added NaH (30 mg, 750.07 umol, 60% purity). The mixture was stirred at 0° C. for 0.5 h, and then 2, 2-difluoroethyl trifluoromethanesulfonate (234 mg, 1.09 mmol) was added to the mixture. The mixture was stirred at 0° C. for 4 h, then poured into ice-water (20 mL) and extracted with ethyl acetate (20 mL×3). The combined organic phases were washed with brine (30 mL), dried with anhydrous Na₂SO₄, filtered, and the filtrate concentrated under reduced pressure to afford the title compound (140 mg, crude) as a colorless oil. MS (ESI): mass calcd. for C₂₀H₂₆F₂N₄O₄, 424.2; m/z found, 425.1 [M+H].

### Intermediate 4. (5R*)-tert-Butyl 5-((2,2-difluoroethoxy)methyl)-5,6,9,10-tetrahydro-4H-isoxazolo[3,4-c]pyrido[4′,3′:3,4]pyrazolo[1,5-a]azepine-11(12H)-carboxylate

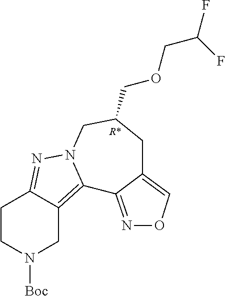 [SMILES: CC(C)(C)OC(=O)N1CCc2nn3c(c2C1)-c1nocc1C[C@@H](COCC(F)F)C3]

The title compound was prepared in a manner analogous to Intermediate 3, but substituting (5R*)-tert-butyl 5-(hydroxymethyl)-5,6,9,10-tetrahydro-4H-isoxazolo[3,4-c]pyrido-[4′,3′:3,4]pyrazolo[1,5-a]azepine-11(12H)-carboxylate for (5S*)-tert-butyl 5-(hydroxy-methyl)-5,6,9,10-tetrahydro-4H-isoxazolo[3,4-c]pyrido[4′,3′:3,4]pyrazolo[1,5-a]azepine-11(12H)-carboxylate in Step B. MS (ESI): mass calcd. for C₂₀H₂₆F₂N₄O₄, 424.2; m/z found, 425.1 [M+H]⁺.

### Intermediate 5: tert-Butyl 5-methylene-5,6,9,10-tetrahydro-4H-isoxazolo[5,4-c]pyrido-[4′,3′:3,4]pyrazolo[1,5-a]azepine-11(12H)-carboxylate

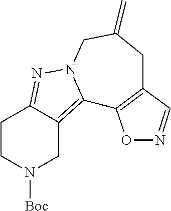 [SMILES: C=C1Cc2cnoc2-c2c3c(nn2C1)CCN(C(=O)OC(C)(C)C)C3]

To a solution of tert-butyl 10-((dimethylamino)methylene)-8-methylene-11-oxo-3,4,8,9,10,11-hexahydro-1H-pyrido[4′,3′:3,4]pyrazolo[1,5-a]azepine-2(7H)-carboxylate (Intermediate 1, product from Step H, 0.32 g, 859.15 umol) in MeOH (10 mL) was added NH₂OH.HCl (358.21 mg, 5.15 mmol). The mixture was stirred at 30° C. for 12 h. The mixture was poured into water (20 mL) and extracted with ethyl acetate (30 mL×3). The combined organic phases were washed with brine (50 mL), dried over anhydrous Na₂SO₄, filtered, and the filtrate concentrated under reduced pressure. The residue was purified by column chromatography (SiO₂, petroleum ether/ethyl acetate=20/1 to 2/1) to afford the title compound (200 mg, 519.87 umol, 89% purity) as a colorless oil. MS (ESI): mass calcd. for C₁₈H₂₂N₄O₃, 342.2; m/z found, 343.1 [M+H]⁺; ¹H NMR (400 MHz, CDCl₃) δ=8.17 (s, 1H), 5.39 (s, 1H), 5.33 (s, 1H), 4.87 (s, 2H), 4.75 (s, 2H), 3.74 (s, 2H), 3.57 (s, 2H), 2.78 (t, J=5.4 Hz, 2H), 1.49 (s, 9H).

### Intermediate 6: tert-Butyl 5-hydroxy-5,6,9,10-tetrahydro-4H-isoxazolo[3,4-c]pyrido[4′,3′:3,4]pyrazolo[1,5-a]azepine-11(12H)-carboxylate

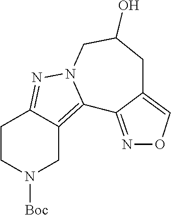 [SMILES: CC(C)(C)OC(=O)N1CCc2nn3c(c2C1)-c1nocc1CC(O)C3]

Step A. tert-Butyl 5-hydroxy-5-(hydroxymethyl)-5,6,9,10-tetrahydro-4H-isoxazolo[3,4-c]pyrido[4′,3′:3,4]pyrazolo[1,5-a]azepine-11(12H)-carboxylate To a solution of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1, product from Step I, 300 mg, 876.19 umol) in THE (20 mL) and H₂O (10 mL) were added NMO (153.97 mg, 1.31 mmol, 138.71 uL) and K₂OsO₄.2H₂O (32.28 mg, 87.62 umol) at 0° C. The mixture was stirred at 25° C. for 16 h. Additional NMO (153.97 mg) and K₂OsO₄.2H₂O (50 mg) were added and the mixture was stirred at 25° C. for 16 h. The mixture was diluted with water (20 mL) and extracted with ethyl acetate (20 mL×3), the combined organic layers were washed with sat. aq. NaHSO₃ (20 mL×2), dried over Na₂SO₄, filtered and the filtrate concentrated under reduced pressure to afford the title compound (334 mg, crude) as a white solid. MS (ESI): mass calcd. for C₁₈H₂₄N₄O₅, 376.2; m/z found, 377.1 [M+H]⁺.

Step B. tert-Butyl 5-oxo-5,6,9,10-tetrahydro-4H-isoxazolo[3,4-c]pyrido[4′,3′:3,4]pyrazolo-[1,5-a]azepine-11(12H)-carboxylate. To a solution of tert-butyl 5-hydroxy-5-(hydroxy-methyl)-5,6,9,10-tetrahydro-4H-isoxazolo[3,4-c]pyrido[4′,3′:3,4]pyrazolo[1,5-a]azepine-11(12H)-carboxylate (330 mg) in THE (3.3 mL) and water (3.3 mL) was added NaIO₄ (562.56 mg, 2.63 mmol, 145.74 uL). The mixture was stirred at 25° C. for 2 h. The mixture was diluted with water (50 mL), extracted with EtOAc (40 mL×2), combined organic layers were dried over Na₂SO₄, filtered, and the filtrate concentrated under reduced pressure to afford the title compound (320 mg, crude) as a brown solid. LCMS indicated 60% of hydrate mass and 24% of desired mass. MS (ESI): mass calcd. for C₁₇H₂₀N₄O₄, 344.4; m/z found, 345.2 [M+H]⁺

Step C. tert-Butyl 5-hydroxy-5,6,9,10-tetrahydro-4H-isoxazolo[3,4-c]pyrido[4′,3′:3,4]-pyrazolo[1,5-a]azepine-11(12H)-carboxylate. To a solution of tert-butyl 5-oxo-5,6,9,10-tetrahydro-4H-isoxazolo[3,4-c]pyrido[4′,3′:3,4]pyrazolo[1,5-a]azepine-11(12H)-carboxylate (300 mg) in EtOH (3 mL) was added NaBH₄ (65.92 mg, 1.74 mmol) at 0° C. The mixture was stirred at 25° C. for 5 h. The reaction was quenched with sat.aq NH₄Cl (20 mL) and extracted with EtOAc (40 mL×3). The combined organic layers were dried over Na₂SO₄, filtered, and the filtrate concentrated under reduced pressure to afford the title compound (230 mg, crude) as a yellow solid. MS (ESI): mass calcd. for C₁₇H₂₂N₄O₄, 346.4; m/z found, 347.3 [M+H]⁺.

### Intermediate 7: (10R)-tert-Butyl 10-methyl-5,6,9,10-tetrahydro-4H-isoxazolo[5,4-c]pyrido[4′,3′:3,4]pyrazolo[1,5-a]azepine-11(12H)-carboxylate

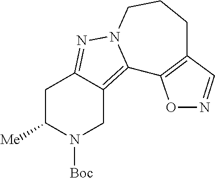 [SMILES: C[C@@H]1Cc2nn3c(c2CN1C(=O)OC(C)(C)C)-c1oncc1CCC3]

Step A. (R)-5-tert-Buty 3-ethyl 2-allyl-6-methyl-6,7-dihydro-2H-pyrazolo[4,3-c]pyridine-3,5(4H)-dicarboxylate. A mixture of (R)-5-tert-butyl 3-ethyl 6-methyl-6,7-dihydro-2H-pyrazolo[4,3-c]pyridine-3,5(4H)-dicarboxylate (preparation as described in PCT Int. Appl. WO 2018005883) (15 g, 48.49 mmol), 3-bromoprop-1-ene (8.80 g, 72.73 mmol), Cs₂CO₃ (39.50 g, 121.22 mmol) in anhydrous DMF (200 mL) was degassed and purged with N₂ 3 times, and then the mixture was stirred at 15° C. for 16 h under N₂ atmosphere. The mixture was poured into water (30 mL) and stirred for 5 min. The aqueous phase was extracted with ethyl acetate (20 mL). The organic phases were washed with brine (30 mL), dried with anhydrous Na₂SO₄, filtered and the filtrate concentrated under reduced pressure. The residue was purified by column chromatography (SiO₂, petroleum ether/ethyl acetate=100/1 to 1/1) to afford the title compound (9.7 g, 26.26 mmol, 54.16% yield, 94.6% purity) as a colorless oil. MS (ESI): mass calcd. for C₁₈H₂₇N₃O₄, 349.2; m/z found, 350.1 [M+H]⁺.

Step B. (R)-tert-Butyl 2-allyl-3-(hydroxymethyl)-6-methyl-6,7-dihydro-2H-pyrazolo[4,3-c]pyridine-5(4H)-carboxylate. A solution of (R)-5-tert-butyl 3-ethyl 2-allyl-6-methyl-6,7-dihydro-2H-pyrazolo[4,3-c]pyridine-3,5(4H)-dicarboxylate (8 g, 22.89 mmol) in THE (80 mL) was added LiAlH₄ (1.30 g, 34.34 mmol) at −40° C. under N₂, and then the mixture was stirred at −40° C. for 2 h under N₂ atmosphere. Ice-NaOH (3 mL, 15% aq) was added to the mixture dropwise at −40° C. and stirred for 5 min. Then the mixture was warmed to 15° C. and filtered. The filtrate was poured into water (40 mL) and extracted with ethyl acetate (30 mL×2). The combined organic phases were washed with brine (100 mL), dried over anhydrous Na₂SO₄, filtered and the filtrate concentrated under reduced pressure. The residue was purified by column chromatography (SiO₂, petroleum ether/ethyl acetate=100/1 to 0:1) to afford the title compound (6.3 g, 20.29 mmol, 88.62% yield, 99% purity) as a colorless oil. MS (ESI): mass calcd. for C₁₆H₂₅N₃O₃, 307.2; m/z found, 308.1 [M+H]⁺.

Step C. (R)-tert-Butyl 2-allyl-3-formyl-6-methyl-6,7-dihydro-2H-pyrazolo[4,3-c]pyridine-5(4H)-carboxylate. To a solution of (COCl)2 (4.74 g, 37.33 mmol, 3.27 mL) in DCM (150 mL) was added DMSO (3.89 g, 49.77 mmol, 3.89 mL) in one portion under N2 at −78° C.

The mixture was stirred at −78° C. for 15 min. Then (R)-tert-butyl 2-allyl-3-(hydroxymethyl)-6-methyl-6,7-dihydro-2H-pyrazolo[4,3-c]pyridine-5(4H)-carboxylate (7.3 g, 24.88 mmol) was added followed by TEA (8.81 g, 87.09 mmol, 12.12 mL). The mixture was stirred at −78° C. for 2 h under a N₂ atmosphere, then the mixture was poured into water (200 mL) at −40° C., stirred for 1 min, then warmed to 15° C. The aqueous phase was extracted with DCM (100 mL×2). The combined organic phases were washed with brine (300 mL), dried over anhydrous Na₂SO₄, filtered and the filtrate concentrated under reduced pressure. The residue was purified by column chromatography (SiO₂, petroleum ether/ethyl acetate=100/1 to 50/1) to afford the title compound (6.4 g, 21.31 mmol, 85.63% yield, 97% purity) as a colorless oil. MS (ESI): mass calcd. for C₁₆H₂₃N₃O₃, 305.2; m/z found, 306.1 [M+H]⁺.

Step D. (6R)-tert-Butyl 2-allyl-3-(1-hydroxybut-3-en-1-yl)-6-methyl-6,7-dihydro-2H-pyrazolo[4,3-c]pyridine-5(4H)-carboxylate. To a solution of (R)-tert-butyl 2-allyl-3-formyl-6-methyl-6,7-dihydro-2H-pyrazolo[4,3-c]pyridine-5(4H)-carboxylate (5.8 g, 18.99 mmol) in THE (60 mL) was added allyl(bromo)magnesium (1 M, 56.98 mL) dropwise at −40° C. under N₂. The mixture was stirred at −40° C. for 30 min, then heated to 0° C. and stirred for 2 h. The mixture was quenched with ice-HCl (aq. 1 N, 50 mL) and stirred for 1 min. The aqueous phase was extracted with ethyl acetate (60 mL×2). The combined organic phases were washed with brine (100 mL), dried with anhydrous Na₂SO₄, filtered and the filtrate concentrated under reduced pressure. The residue was purified by column chromatography (SiO₂, petroleum ether/ethyl acetate=100/1 to 1:1) to afford the title compound (5.7 g, 15.70 mmol, 82.66% yield, 95.7% purity) as a colorless oil. MS (ESI): mass calcd. for C₁₉H₂₉N₃O₃, 347.2; m/z found, 348.1 [M+H]⁺.

Step E. (3R)-tert-Butyl 11-hydroxy-3-methyl-3,4,10,11-tetrahydro-1H-pyrido[4′,3′:3,4]-pyrazolo[1,5-a]azepine-2(7H)-carboxylate. A mixture of (6R)-tert-butyl 2-allyl-3-(1-hydroxybut-3-en-1-yl)-6-methyl-6,7-dihydro-2H-pyrazolo[4,3-c]pyridine-5(4H)-carboxylate (2.2 g, 6.33 mmol), [1,3-bis(2,4,6-trimethylphenyl)imidazolidin-2-ylidene]-dichloro-[(2-isopropoxyphenyl)methylene] ruthenium (396.77 mg, 633.18 umol) in DCM (1.6 L) was degassed and purged with N₂ (3×), and then the mixture was stirred at 40° C. for 16 h under a N₂ atmosphere. [1,3-Bis(2,4,6-trimethylphenyl)imidazolidin-2-ylidene]-dichloro-[(2-isopropoxyphenyl)methylene]ruthenium (198.38 mg, 316.59 umol) was added to the mixture at 15° C. under a nitrogen atmosphere. The mixture was stirred at 34° C. for another 32 h under N₂, then the mixture was stirred at 40° C. for an additional 32 h. The mixture was concentrated under reduced pressure. The residue was purified by column chromatography (SiO₂, petroleum ether/ethyl acetate=100/1 to 3/1) to afford the title compound (1.8 g, 5.58 mmol, 88.11% yield, 99% purity) as a black brown solid. MS (ESI): mass calcd. for C₁₇H₂₅N₃O₃, 319.2; m/z found, 320.1 [M+H]⁺.

Step F. (3R)-tert-Butyl 11-hydroxy-3-methyl-3,4,8,9,10,11-hexahydro-1H-pyrido-[4′,3′:3,4]pyrazolo[1,5-a]azepine-2(7H)-carboxylate. To a solution of (3R)-tert-butyl 11-hydroxy-3-methyl-3,4,10,11-tetrahydro-1H-pyrido[4′,3′:3,4]pyrazolo[1,5-a]azepine-2(7H)-carboxylate (750 mg, 2.35 mmol) in MeOH (30 mL) was added Pd/C (75 mg, 10%) under N₂. The suspension was degassed under reduced pressure and purged with H₂ several times. The mixture was stirred under H₂ (15 psi) at 15° C. for 16 h. The mixture was filtered and concentrated under reduced pressure to afford the title compound (680 mg, 2.12 mmol, 90.10% yield) as a black brown oil. MS (ESI): mass calcd. for C₁₇H₂₇N₃O₃, 321.2; m/z found, 322.1 [M+H]⁺.

### Step G. (R)-tert-Butyl 3-methyl-11-oxo-3,4,8,9,10,11-hexahydro-1H-pyrido[4′,3′:3,4]pyrazolo[1,5-a]azepine-2(7H)-carboxylate

A mixture of (3R)-tert-butyl 11-hydroxy-3-methyl-3,4,8,9,10,11-hexahydro-1H-pyrido-[4′,3′:3,4]pyrazolo[1,5-a]azepine-2(7H)-carboxylate (680 mg, 2.12 mmol), TPAP (148.70 mg, 423.13 umol) and NMO (991.36 mg, 8.46 mmol, 893.11 uL) in acetonitrile (ACN) (10 mL) was degassed and purged with N₂ (3×), and then the mixture was stirred at 15° C. for 16 h under a N₂ atmosphere. The mixture was concentrated under reduced pressure. The residue was purified by column chromatography (SiO₂, petroleum ether/ethyl acetate=10/1 to 1/1) to afford the title compound (600 mg, 1.84 mmol, 87.02% yield, 98% purity) as a yellow oil. MS(ESI): mass calcd. for C₁₇H₂₅N₃O₃, 319.2; m/z found, 320.1 [M+H]⁺.

### Step H. (R)-tert-Butyl 10-(hydroxymethylene)-3-methyl-11-oxo-3,4,8,9,10,11-hexahydro-1H-pyrido[4′,3′:3,4]pyrazolo[1,5-a]azepine-2(7H)-carboxylate

A solution of (R)-tert-butyl 3-methyl-11-oxo-3,4,8,9,10,11-hexahydro-1H-pyrido-[4′,3′:3,4]pyrazolo[1,5-a]azepine-2(7H)-carboxylate (400 mg, 1.25 mmol) in DMF-DMA (13.46 g, 112.91 mmol, 15 mL) was stirred at 75° C. for 16 h. The mixture was stirred at 75° C. for 16 h. The reaction mixture was concentrated under reduced pressure. The residue was poured into water (20 mL) and stirred for 2 min. The aqueous phase was extracted with ethyl acetate (20 mL×2). The combined organic phases were washed with brine (10 mL×2), dried with anhydrous Na₂SO₄, filtered and the filtrate concentrated under reduced pressure to afford the title compound (440 mg, crude) as a yellow solid. MS (ESI): mass calcd. for C₁₈H₂₅N₃O₄, 347.2; m/z found, 348.1 [M+H]⁺.

### Step I. (10R)-tert-Butyl 10-methyl-5,6,9,10-tetrahydro-4H-isoxazolo[5,4-c]pyrido [4′,3′:3,4]pyrazolo[1,5-a]azepine-11(12H)-carboxylate

To a solution of (R)-tert-butyl 10-(hydroxymethylene)-3-methyl-11-oxo-3,4,8,9,10,11-hexahydro-1H-pyrido[4′,3′:3,4]pyrazolo[1,5-a]azepine-2(7H)-carboxylate (200 mg, 575.69 umol) in MeOH (30 mL) was added NH₂OH.HCl (240.03 mg, 3.45 mmol) in one portion at 30° C. under N₂. The mixture was stirred at 30° C. for 16 h. The mixture was poured into water (100 mL) and stirred for 1 min. The aqueous phase was extracted with ethyl acetate (50 mL×2). The combined organic phases were washed with brine (100 mL×2), dried with anhydrous Na₂SO₄, filtered and the filtrate concentrated under reduced pressure. The residue was purified by prep-TLC (petroleum ether/ethyl acetate=1/2) to afford the title compound (120 mg, 348.42 umol, 60.52% yield) as a light yellow oil. MS (ESI): mass calcd. for C₁₈H₂₄N₄O₃, 344.2; m/z found, 345.1 [M+H]⁺.

### Intermediate 8: (11R)-tert-Butyl 11-methyl-6,7,10,11-tetrahydro-5H-pyrido[2,3-c]-pyrido[4′,3′:3,4]pyrazolo[1,5-a]azepine-12(13H)-carboxylate

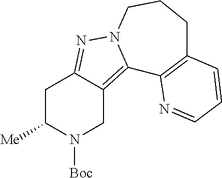 [SMILES: C[C@@H]1Cc2nn3c(c2CN1C(=O)OC(C)(C)C)-c1ncccc1CCC3]

To a solution of (R)-tert-butyl 3-methyl-11-oxo-3,4,8,9,10,11-hexahydro-1H-pyrido-[4′,3′:3,4]pyrazolo[1,5-a]azepine-2(7H)-carboxylate (160 mg, 500.94 umol, product of Step G in Intermediate 7) and prop-2-yn-1-amine (137.96 mg, 2.50 mmol, 160.41 uL) in EtOH (2 mL) was added NaAuCl₄.2H₂O (49.82 mg, 125.24 umol). The mixture was stirred at 80° C. for 72 h. The residue was diluted with water (10 mL) and the mixture was extracted with EtOAc (10 mL×3). The combined organic layers were washed with brine (10 mL), dried over Na₂SO₄, filtered and the filtrate concentrated under reduced pressure. The residue was purified by column chromatography (SiO₂, petroleum ether/ethyl acetate=10/1 to 2/1) to give the title compound (90 mg, 190.44 umol, 38.02% yield, 75% purity) as a yellow oil. MS (ESI): mass calcd. for C₂₀H₂₆N₄O₂, 354.2; m/z found, 355.1 [M+H]⁺.

### Intermediate 9: (10R)-tert-Butyl 10-methyl-5,6,9,10-tetrahydro-4H-isoxazolo[3,4-c]pyrido[4′,3′:3,4]pyrazolo[1,5-a]azepine-11(12H)-carboxylate

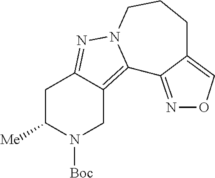 [SMILES: C[C@@H]1Cc2nn3c(c2CN1C(=O)OC(C)(C)C)-c1nocc1CCC3]

To a solution of (R)-tert-butyl 10-(hydroxymethylene)-3-methyl-11-oxo-3,4,8,9,10,11-hexahydro-1H-pyrido[4′,3′:3,4]pyrazolo[1,5-a]azepine-2(7H)-carboxylate (200 mg, 575.69 umol) in Py (30 mL) was added NH₂OH.HCl (240.03 mg, 3.45 mmol) in one portion under N₂. The mixture was stirred at 115° C. for 16 h. The mixture was concentrated under reduced pressure. The residue was poured into HCl (1N aq, 100 mL) and stirred for 1 min. The aqueous phase was extracted with ethyl acetate (50 mL×2). The combined organic phases were washed with brine (100 mL×2), dried over anhydrous Na₂SO₄, filtered, and the filtrate concentrated under reduced pressure. The residue was purified by prep-TLC (petroleum ether/ethyl acetate=1/2) to afford the title compound (90 mg, 261.32 umol, 45.39% yield) as a light yellow oil. MS (ESI): mass calcd. for C₁₈H₂₄N₄O₃, 344.2; m/z found, 345.1 [M+H]⁺.

### Intermediate 10: tert-Butyl 6,7,10,11-tetrahydro-5H-pyrido[4′,3′:3,4]pyrazolo[1,5-a][1,2,4]triazolo[3,4-c][1,4]diazepine-12(13H)-carboxylate

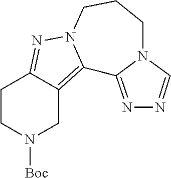 [SMILES: CC(C)(C)OC(=O)N1CCc2nn3c(c2C1)-c1nncn1CCC3]

Step A. tert-Butyl 11-thioxo-3,4,8,9,10,11-hexahydro-1H-pyrido[4′,3′:3,4]pyrazolo[1,5-a][1,4]diazepine-2(7H)-carboxylate. To a solution of tert-butyl 11-oxo-3,4,7,8,9,10-hexahydro-1H-pyrido[2,3]pyrazolo [2,4-b][1,4]diazepine-2-carboxylate (preparation as described in PCT Int. Appl. WO2018005883, Jan. 4, 2018) (250.00 mg, 816.03 umol) in toluene (5 mL) was added Lawesson's reagent (165.03 mg, 408.02 umol). The mixture was heated to 110° C. for 3 h, then concentrated under reduced pressure. The residue was purified by prep-TLC (EtOAc) to afford the title compound (258.00 mg, 800.20 umol, 98.06% yield) as a yellow solid.

Step B. tert-Butyl 6,7,10,11-tetrahydro-5H-pyrido[4′,3′:3,4]pyrazolo[1,5-a][1,2,4]triazolo-[3,4-c][1,4]diazepine-12(13H)-carboxylate. To a solution of tert-butyl 11-thioxo-3,4,8,9,10,11-hexahydro-1H-pyrido[4′,3′:3,4]pyrazolo[1,5-a][1,4]diazepine-2(7H)-carboxylate (80.00 mg, 248.12 umol) and formohydrazide (74.51 mg, 1.24 mmol) in MeCN (3.00 mL) was added Hg(OAc)₂ (118.61 mg, 372.18 umol), then the mixture was stirred at 20° C. for 16 h. The mixture was diluted with water (50 mL) and extracted with EtOAc (20 mL×3). The combined organic layers were washed with brine (20 mL×2), dried over Na₂SO₄, filtered, and the filtrate concentrated under reduced pressure to afford the title compound (100.00 mg, crude) as a colorless oil.

### Intermediate 11: tert-Butyl 3-methyl-6,7,10,11-tetrahydro-5H-pyrido[4′,3′:3,4]pyrazolo[1,5-a][1,2,4]triazolo[3,4-c][1,4]diazepine-12(13H)-carboxylate

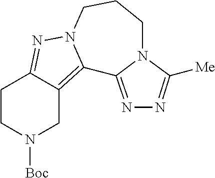 [SMILES: Cc1nnc2n1CCCn1nc3c(c1-2)CN(C(=O)OC(C)(C)C)CC3]

Step A. tert-Butyl 11-thioxo-3,4,8,9,10,11-hexahydro-1H-pyrido[4′,3′:3,4]pyrazolo[1,5-a][1,4]diazepine-2(7H)-carboxylate. The title compound was prepared in a manner analogous to Intermediate 12, using tert-butyl 11-oxo-3,4,8,9,10,11-hexahydro-1H-pyrido[4′,3′:3,4]pyrazolo[1,5-a][1,4]diazepine-2(7H)-carboxylate (preparation as described in PCT Int. Appl. WO 2018005883) instead of (R)-tert-butyl 3-methyl-11-oxo-3,4,8,9,10,11-hexahydro-1H-pyrido[4′,3′:3,4]pyrazolo[1,5-a][1,4]diazepine-2(7H)-carboxylate in Step A. The title compound was used directly in the next step without further purification.

Step B: tert-Butyl 3-methyl-6,7,10,11-tetrahydro-5H-pyrido[4′,3′:3,4]pyrazolo[1,5-a][1,2,4]triazolo[3,4-c][1,4]diazepine-12(13H)-carboxylate. To a solution of tert-butyl 11-thioxo-3,4,8,9,10,11-hexahydro-1H-pyrido[4′,3′:3,4]pyrazolo[1,5-a][1,4]diazepine-2(7H)-carboxylate (80.00 mg, 248.12 umol) and acetohydrazide (91.90 mg, 1.24 mmol) in MeCN (3.00 mL) was added Hg(OAc)₂ (118.61 mg, 372.18 umol), then the mixture was stirred at 20° C. for 16 h. The mixture was extracted with EtOAc (20 mL×3) and water (30 mL). The combined organic layers were washed with brine (20 mL×2), dried over Na₂SO₄, filtered and the filtrate concentrated under reduced pressure to afford the title compound (100.00 mg, crude) as a colorless oil.

### Intermediate 12: (11R)-tert-Butyl 11-methyl-6,7,10,11-tetrahydro-5H-pyrido[4′,3′:3,4]pyrazolo[1,5-a][1,2,4]triazolo[3,4-c][1,4]diazepine-12(13H)-carboxylate

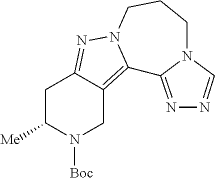 [SMILES: C[C@@H]1Cc2nn3c(c2CN1C(=O)OC(C)(C)C)-c1nncn1CCC3]

Step A. (R)-tert-Butyl 3-methyl-11-thioxo-3,4,8,9,10,11-hexahydro-1H-pyrido[4′,3′:3,4]-pyrazolo[1,5-a][1,4]diazepine-2(7H)-carboxylate. To a solution of (R)-tert-butyl 3-methyl-11-oxo-3,4,8,9,10,11-hexahydro-1H-pyrido[4′,3′:3,4]pyrazolo[1,5-a][1,4]diazepine-2(7H)-carboxylate (preparation as described in PCT Int. Appl. WO 2018005883) (300.00 mg, 936.36 umol) in toluene (3.00 mL) was added Lawesson reagent (189.36 mg, 468.18 umol). The mixture was heated to 110° C. for 3 h, then concentrated under reduced pressure. The residue was purified by column chromatography (petroleum ether/ethyl acetate=30% to 50%) to afford the title compound (270.00 mg, 650.02 umol, 69.42% yield, 81% purity) as a yellow solid.

Step B. (11R)-tert-Butyl 11-methyl-6,7,10,11-tetrahydro-5H-pyrido[4′,3′:3,4]pyrazolo[1,5-a][1,2,4]triazolo[3,4-c][1,4]diazepine-12(13H)-carboxylate. To a suspension of (R)-tert-butyl 3-methyl-11-thioxo-3,4,8,9,10,11-hexahydro-1H-pyrido[4′,3′:3,4]pyrazolo[1,5-a][1,4]-diazepine-2(7H)-carboxylate (100.00 mg, 297.22 umol) and formohydrazide (89.26 mg, 1.49 mmol) in MeCN (2.00 mL) was added Hg(OAc)₂ (142.08 mg, 445.83 umol). The mixture was stirred at 25° C. for 16 h, then diluted with water (50 mL) and extracted with EtOAc (50 mL×3). The combined organic layers were washed with brine (50 mL×2), dried over Na₂SO₄, filtered, and the filtrate concentrated under reduced pressure to afford the title compound (90.00 mg, crude) as a white solid. MS (ESI): mass calcd. for C₁₇H₂₄N₆O₂, 344.2; m/z found, 345.0 [M+H]⁺.

### Intermediate 13: (11R)-tert-Butyl 3,11-dimethyl-6,7,10,11-tetrahydro-5H-pyrido[4′,3′:3,4]pyrazolo[1,5-a][1,2,4]triazolo[3,4-c][1,4]diazepine-12(13H)-carboxylate

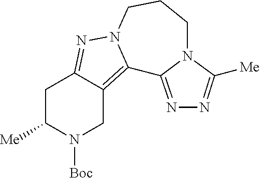 [SMILES: Cc1nnc2n1CCCn1nc3c(c1-2)CN(C(=O)OC(C)(C)C)[C@H](C)C3]

To a suspension of (R)-tert-butyl 3-methyl-11-thioxo-3,4,8,9,10,11-hexahydro-1H-pyrido[4′,3′:3,4]pyrazolo[1,5-a][1,4]diazepine-2(7H)-carboxylate (Intermediate 12, product from Step A, 80.00 mg, 237.78 umol) and acetohydrazide (88.07 mg, 1.19 mmol) in MeCN (3.00 mL) was added Hg(OAc)₂ (113.66 mg, 356.67 umol). The mixture was stirred at 25° C. for 16 h, then diluted with water (50 mL) and extracted with EtOAc (50 mL×3). The combined organic layers were washed with brine (50 mL×2), dried over Na₂SO₄, filtered, and the filtrate concentrated under reduced pressure to afford the title compound (90.00 mg, crude) as a white solid.

### Intermediate 14: tert-Butyl 11-oxo-3,4,8,9,10,11-hexahydro-1H-pyrido[4′,3′:3,4]pyrazolo-[1,5-a]azepine-2(7H)-carboxylate

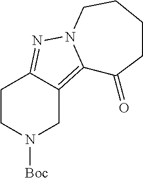 [SMILES: CC(C)(C)OC(=O)N1CCc2nn3c(c2C1)C(=O)CCCC3]

Step A. 5-tert-Butyl 3-ethyl 2-allyl-6,7-dihydro-2H-pyrazolo[4,3-c]pyridine-3,5(4H)-dicarboxylate. To a mixture of 5-tert-butyl 3-ethyl 6,7-dihydro-2H-pyrazolo[4,3-c]pyridine-3,5 (4H)-dicarboxylate (preparation as described in WO2018005881, publication date Jan. 4, 2018) (5.00 g, 16.93 mmol) and 3-bromoprop-1-ene (3.07 g, 25.40 mmol) in DMF (50.00 mL) was added Cs₂CO₃ (13.79 g, 42.33 mmol) in one portion under N₂. The mixture was stirred at 50° C. for 12 h. The mixture was poured into water (50 mL) and stirred for 1 min. The aqueous phase was extracted with ethyl acetate (50 mL×2). The combined organic phases were washed with brine (50 mL×2), dried with anhydrous Na₂SO₄, filtered, and the filtrate concentrated under reduced pressure. The residue was purified by silica gel chromatography (petroleum ether/ethyl acetate=15/1 to 5/1) to afford the title compound (2.70 g, 7.89 mmol, 46.60% yield, 98% purity) as a yellow solid. MS (ESI): mass calcd. for C₁₇H₂₅N₃O₄, 335.1; m/z found, 336.0 [M+H]⁺.

Step B. tert-Butyl 2-allyl-3-(hydroxymethyl)-6,7-dihydro-2H-pyrazolo[4,3-c]pyridine-5(4H)-carboxylate. To a mixture of 5-tert-butyl 3-ethyl 2-allyl-6,7-dihydro-2H-pyrazolo[4,3-c]pyridine-3,5(4H)-dicarboxylate (1.00 g, 2.98 mmol) in THE (30.00 mL) was added LiAlH₄ (169.72 mg, 4.47 mmol) in one portion at −40° C. under N₂. The mixture was stirred at 20° C. for 1 h. The mixture was quenched with HCl (1N aq 10 mL). The aqueous phase was extracted with ethyl acetate (20 mL×2). The combined organic phases were washed with brine (20 mL×2), dried with anhydrous Na₂SO₄, filtered, and the filtrate concentrated under reduced pressure. The residue was purified by silica gel chromatography (dichloromethane/methanol=100/1-20/1) to afford the title compound (780.00 mg, 2.66 mmol, 89.22% yield) as a yellow solid. MS (ESI): mass calcd. for C₁₅H₂₃N₃O₃, 293.1; m/z found, 294 [M+H]⁺.

Step C. tert-Butyl 2-allyl-3-formyl-6,7-dihydro-2H-pyrazolo[4,3-c]pyridine-5(4H)-carboxylate. To a mixture of tert-butyl 2-allyl-3-(hydroxymethyl)-6,7-dihydro-2H-pyrazolo [4,3-c]pyridine-5(4H)-carboxylate (780.00 mg, 2.66 mmol) in DCM (30.00 mL) was added MnO₂ (2.31 g, 26.60 mmol) in one portion under N₂. The mixture was stirred at 45° C. for 12 h. Additional MnO₂ (2.31 g, 26.60 mmol) was added and the mixture was stirred at 45° C. for another 24 h. At this time the mixture was filtered and concentrated under reduced pressure. The residue was purified by silica gel chromatography (petroleum ether/ethyl acetate=10/1 to 3/1) to afford the title compound (450.00 mg, 1.54 mmol, 58.07% yield, 100% purity) as a yellow solid. MS (ESI): mass calcd. for C₁₅H₂₁N₃O₃, 291.1; m/z found, 292 [M+H]⁺.

Step D. tert-Butyl 2-allyl-3-(1-hydroxybut-3-en-1-yl)-6,7-dihydro-2H-pyrazolo[4,3-c]pyridine-5(4H)-carboxylate. To a mixture of tert-butyl 2-allyl-3-formyl-6,7-dihydro-2H-pyrazolo[4,3-c]pyridine-5(4H)-carboxylate (800.00 mg, 2.75 mmol) in THE (5.00 mL) was added allyl(bromo)magnesium (1 M, 8.24 mL) in one portion at −40° C. under N₂. The mixture was stirred at −40° C. for 2 h. The mixture was poured into water (20 mL) and stirred for 1 min. The aqueous phase was extracted with ethyl acetate (20 mL×2). The combined organic phases were washed with brine (10 mL×2), dried with anhydrous Na₂SO₄, filtered, and the filtrate concentrated under reduced pressure. The residue was purified by silica gel chromatography (petroleum ether/ethyl acetate=3/1 to 1/1) to afford the title compound (750.00 mg, 2.16 mmol, 78.53% yield, 96% purity) as a yellow oil. MS (ESI): mass calcd. for C₁₈H₂₇N₃O₃, 333.2; m/z found, 334 [M+H]⁺.

Step E. tert-Butyl 11-hydroxy-3,4,10,11-tetrahydro-1H-pyrido[4′,3′:3,4]pyrazolo [1,5-a]azepine-2(7H)-carboxylate. To a mixture of tert-butyl 2-allyl-3-(1-hydroxybut-3-en-1-yl)-6,7-dihydro-2H-pyrazolo[4,3-c]pyridine-5(4H)-carboxylate (750.00 mg, 2.25 mmol) in DCM (1.20 L) was added benzylidene-[1,3-bis(2,4,6-trimethylphenyl)imidazolidin-2-ylidene]-dichloro-ruthenium; tricyclohexylphosphane (381.94 mg, 449.88 umol) in one portion under N₂. The mixture was stirred at 30° C. for 12 h. The mixture was concentrated under reduced pressure. The residue was purified by silica gel chromatography (petroleum ether/ethyl acetate=4/1 to 1/1) to afford the title compound (650.00 mg, 2.02 mmol, 89.87% yield, 95% purity) as a yellow solid. MS (ESI): mass calcd. for C₁₆H₂₃N₃O₃, 305.1; m/z found, 306 [M+H]⁺.

Step F. tert-Butyl 11-hydroxy-3,4,8,9,10,11-hexahydro-1H-pyrido[4′,3′:3,4] pyrazolo[1,5-a]azepine-2(7H)-carboxylate. To a solution of tert-butyl 11-hydroxy-3,4,10,11-tetrahydro-1H-pyrido[4′,3′:3,4] pyrazolo[1,5-a]azepine-2(7H)-carboxylate (150.00 mg, 491.21 umol) in MeOH (5.00 mL) was added Pd/C (20.00 mg, 10%) under N₂. The suspension was degassed under reduced pressure and purged with H₂ several times. The mixture was stirred under H₂ (15 psi) at 30° C. for 12 h. The reaction mixture was filtered, and the filtrate was concentrated to afford the title compound (140.00 mg, 455.45 umol, 92.72% yield) as a yellow solid. MS (ESI): mass calcd. for C₁₆H₂₅N₃O₃, 307.1; m/z found, 308 [M+H]⁺.

Step G. tert-Butyl 11-oxo-3,4,8,9,10,11-hexahydro-1H-pyrido[4′,3′:3,4]pyrazolo[1,5-a]azepine-2(7H)-carboxylate. To a mixture of tert-butyl 11-hydroxy-3,4,8,9,10,11-hexahydro-1H-pyrido [4′,3′:3,4]pyrazolo[1,5-a]azepine-2(7H)-carboxylate (2.00 g, 6.51 mmol) in MeCN (80.00 mL) was added NMO (3.05 g, 26.04 mmol, 2.75 mL) and TPAP (457.31 mg, 1.30 mmol) in one portion under N₂. The mixture was stirred at 30° C. for 12 h. The mixture was filtered and concentrated under reduced pressure. The residue was purified by silica gel chromatography (petroleum ether/ethyl acetate=4/1 to 1/1) to afford the title compound (1.60 g, 5.24 mmol, 80.48% yield) as a yellow oil. MS (ESI): mass calcd. for C₁₆H₂₃N₃O₃, 305.1; m/z found, 306 [M+H].

### Intermediate 15: tert-Butyl 6,7,10,11-tetrahydro-5H-pyridazino[3,4-c]pyrido[4′,3′:3,4]-pyrazolo[1,5-a]azepine-12(13H)-carboxylate

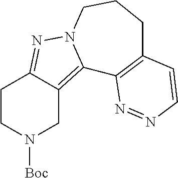 [SMILES: CC(C)(C)OC(=O)N1CCc2nn3c(c2C1)-c1nnccc1CCC3]

Step A. tert-Butyl 10-allyl-11-oxo-3,4,8,9,10,11-hexahydro-1H-pyrido[4′,3′:3,4]pyrazolo[1,5-a]azepine-2(7H)-carboxylate. To a solution of tert-butyl 11-oxo-3,4,8,9,10,11-hexahydro-1H-pyrido[4′,3′:3,4]pyrazolo[1,5-a]azepine-2(7H)-carboxylate (Intermediate 14, 300.00 mg, 982.41 umol) and HMPA (440.12 mg, 2.46 mmol, 431.49 uL) in THE (8.00 mL) at −78° C. was added LDA (6 mL, 1.25 M, freshly prepared from N-isopropylpropan-2-amine (1.22 g, 12.05 mmol, 1.69 mL) in THE (3.00 mL) by adding n-BuLi (2.5 M, 5.00 mL) at −65° C.), then warm to −30° C. for 0.5 h. 3-Bromoprop-1-ene (594.26 mg, 4.91 mmol) was added at −78° C. The mixture was warmed to 30° C. and stirred for another 1 h. The reaction was quenched with HCl (1 N aq, 10 mL) and extracted with EtOAc (20 mL×3). The combined organic layers were dried over anhydrous Na₂SO₄, filtered, and the filtrate concentrated under reduced pressure. The residue was purified by prep-TLC (petroleum ether/ethyl acetate=1/1) and further purified by RP HPLC (Condition A) to afford tert-butyl 10-allyl-11-oxo-3,4,8,9,10,11-hexahydro-1H-pyrido[4′,3′:3,4]pyrazolo[1,5-a]azepine-2(7H)-carboxylate (31.00 mg, 89.74 umol, 9.14% yield) as a colorless oil. MS (ESI): mass calcd. for C₁₉H₂₇N₃O₃, 345.2; m/z found, 346.1 [M+H]⁺.

Step B. tert-Butyl 11-oxo-10-(2-oxoethyl)-3,4,8,9,10,11-hexahydro-1H-pyrido 20 [4′,3′:3,4]pyrazolo[1,5-a]azepine-2(7H)-carboxylate. To a mixture of tert-butyl 10-allyl-11-oxo-3,4,7,8,9,10-hexahydro-1H-pyrido[2,3]pyrazolo[2,4-b]azepine-2-carboxylate (60.00 mg, 173.70 umol) in THE (4.00 mL) and H₂O (4.00 mL) was added OsO₄ (13.25 mg, 52.11 umol, 2.70 uL) and NaIO₄ (148.61 mg, 694.80 umol, 38.50 uL) in one portion at 0° C. under N₂. The mixture was stirred at 20° C. for 10 h. The mixture was poured into water (10 mL) and stirred for 1 min. The aqueous phase was extracted with ethyl acetate (10 mL×2). The combined organic phases were washed with brine (5 mL×2), dried over anhydrous Na₂SO₄, filtered and the filtrate concentrated under reduced pressure to afford the title compound (60.35 mg, crude) as a yellow oil. MS(ESI): mass calcd. for C18H₂₅N₃O₄, 347.1; m/z found, 348.1 [M+H]⁺.

Step C. tert-Butyl 4a,5,6,7,10,11-hexahydro-4H-pyridazino[3,4-c]pyrido [4′,3′:3,4]pyrazolo-[1,5-a]azepine-12(13H)-carboxylate. To a mixture of tert-butyl 11-oxo-10-(2-oxoethyl)-3,4,7,8,9,10-hexahydro-1H-pyrido[2,3]pyrazolo[2,4-b]azepine-2-carboxylate (60.35 mg, 173.71 umol) in EtOH (10.00 mL) was added N₂H₄.H₂O (15.35 mg, 260.57 umol, 14.90 uL, 85% purity) in one portion at 0° C. under N₂. The mixture was stirred at 20° C. for 2 h. The reaction mixture was used in the next step directly. MS (ESI): mass calcd. for C18H₂₅N₅O₂, 343.2; m/z found, 344.1 [M+H]⁺.

Step D. tert-Butyl 6,7,10,11-tetrahydro-5H-pyridazino[3,4-c]pyrido[4′,3′:3,4] pyrazolo[1,5-a]azepine-12(13H)-carboxylate. To the reaction mixture from Step C was added DDQ (47.32 mg, 208.46 umol) under N₂. The mixture was stirred at 0° C. for 2 h. The mixture was concentrated under reduced pressure. The residue was purified by prep-TLC (DCM/MeOH=10/1) to afford the title compound (17.00 mg, 48.95 umol, 28.18% yield, 98.3% purity) as a yellow oil. MS(ESI): mass calcd. for C₁₈H₂₃N₅O₂, 341.1; m/z found, 342 [M+H]⁺.

### Intermediate 16: tert-Butyl 4,5,6,9,10,12-hexahydropyrazolo[3,4-c]pyrido[4′,3′:3,4]pyrazolo-[1,5-a]azepine-11(2H)-carboxylate

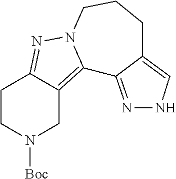 [SMILES: CC(C)(C)OC(=O)N1CCc2nn3c(c2C1)-c1n[nH]cc1CCC3]

Step A. tert-Butyl 10-(hydroxymethylene)-11-oxo-3,4,8,9,10,11-hexahydro-1H-pyrido[4′,3′:3,4]pyrazolo[1,5-a]azepine-2(7H)-carboxylate. A mixture of tert-butyl 11-oxo-3,4,7,8,9,10-hexahydro-1H-pyrido[2,3]pyrazolo [2,4-a]azepine-2-carboxylate (200.00 mg, 654.94 umol, Intermediate 14) in DMF-DMA (18.00 g, 151.07 mmol, 20.00 mL) was stirred at 75° C. for 12 h. The mixture was stirred at 75° C. for another 24 h, then concentrated under reduced pressure. The residue was poured into water (20 mL) and stirred for 2 min. The aqueous phase was extracted with ethyl acetate (20 mL×2). The combined organic phases were washed with brine (10 mL×2), dried with anhydrous Na₂SO₄, filtered and the filtrate concentrated under reduced pressure to afford title compound (210.00 mg, 629.91 umol, 96.18% yield) as a yellow solid. MS(ESI): mass calcd. for C₁₇H₂₃N₃O₄, 333.1; m/z found, 334.1 [M+H]⁺.

Step B. tert-Butyl 4,5,6,9,10,12-hexahydropyrazolo[3,4-c]pyrido[4′,3′:3,4]pyrazolo[1,5-a]azepine-11(2H)-carboxylate. To a mixture of tert-butyl 10-(hydroxymethylene)-11-oxo-3,4,8,9,10,11-hexahydro-1H-pyrido[4′,3′:3,4]pyrazolo[1,5-a]azepine-2(7H)-carboxylate (80.00 mg, 239.97 umol) in MeOH (5.00 mL) was added N₂H₄.H₂O (28.27 mg, 479.93 umol, 27.44 uL, 85% purity) in one portion at 30° C. under N₂. The mixture was stirred at 30° C. for 10 h. The mixture was concentrated under reduced pressure. The residue was purified by prep-TLC (petroleum ether/ethyl acetate=1/2) to afford the title compound (54.00 mg, 163.93 umol, 68.31% yield) as a yellow solid. MS (ESI): mass calcd. for C₁₇H₂₃N5O₂, 329.1; m/z found, 330.1 [M+H]⁺.

### Intermediate 17: tert-Butyl 6,7,10,11-tetrahydro-5H-pyrido[2,3-c]pyrido[4′,3′:3,4]pyrazolo-[1,5-a]azepine-12(13H)-carboxylate

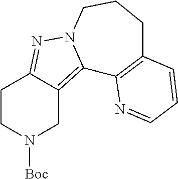 [SMILES: CC(C)(C)OC(=O)N1CCc2nn3c(c2C1)-c1ncccc1CCC3]

The title compound was prepared in a manner analogous to Intermediate 8, substituting tert-butyl-11-oxo-3,4,8,9,10,11-hexahydro-1H-pyrido[4′,3′:3,4]pyrazolo[1,5-a]azepine-2(7H)-carboxylate for (R)-tert-butyl 3-methyl-11-oxo-3,4,8,9,10,11-hexahydro-1H-pyrido[4′,3′:3,4]pyrazolo[1,5-a]azepine-2(7H)-carboxylate. MS (ESI): mass calcd. for C₁₉H₂₄N₄O₂, 340.2; m/z found, 341.0 [M+H]⁺.

### Intermediate 18: tert-Butyl 2-methyl-4,5,6,9,10,12-hexahydropyrazolo[3,4-c]pyrido-[4′,3′:3,4]pyrazolo[1,5-a]azepine-11(2H)-carboxylate

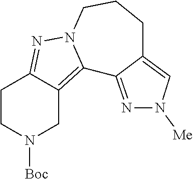 [SMILES: Cn1cc2c(n1)-c1c3c(nn1CCC2)CCN(C(=O)OC(C)(C)C)C3]

To a mixture of tert-butyl 10-(hydroxymethylene)-11-oxo-3,4,8,9,10,11-hexahydro-1H-pyrido[4′,3′:3,4]pyrazolo[1,5-a]azepine-2(7H)-carboxylate (130.00 mg, 389.95 umol, Intermediate 15 product from Step A) in MeOH (5.00 mL) was added methylhydrazine (89.82 mg, 779.90 umol, 102.07 uL) in one portion at 30° C. under N₂. The mixture was stirred at 30° C. for 10 h. The mixture was concentrated under reduced pressure, then purified by prep-TLC (petroleum ether/ethyl acetate=1/2) to afford 100 mg of crude product, which was further purified by RP HPLC (Condition A) to afford title compound tert-butyl 2-methyl-4,5,6,9,10,12-hexahydropyrazolo[3,4-c]pyrido[4′,3′:3,4]pyrazolo [1,5-a]azepine-11(2H)-carboxylate (70.00 mg, 203.83 umol, 52.27% yield) as a yellow solid, and another regioisomer tert-butyl 1-methyl-4,5,6,9,10,12-hexahydropyrazolo[3,4-c]pyrido[4′,3′:3,4]-pyrazolo[1,5-a]azepine-11(1H)-carboxylate (20.00 mg, 58.24 umol, 14.93% yield) as a yellow solid. MS(ESI): mass calcd. for C₁₈H₂₅N₅O₂, 343.2; m/z found, 344.2 [M+H]⁺; ¹H NMR (400 MHz, CDCl₃) δ 7.19 (s, 1H), 4.63-4.77 (m, 2H), 4.38-4.53 (m, 2H), 4.06-4.20 (m, 1H), 3.84-3.94 (m, 3H), 3.84-3.94 (m, 3H), 3.64-3.67 (m, 1H), 3.72 (br s, 1H), 2.83-2.96 (m, 2H), 2.76 (br t, J=5.58 Hz, 2H), 2.13-2.30 (m, 2H), 1.50 (s, 9H).

### Intermediate 19: tert-Butyl 1-methyl-4,5,6,9,10,12-hexahydropyrazolo[3,4-c]pyrido-[4′,3′:3,4]pyrazolo[1,5-a]azepine-11(1H)-carboxylate

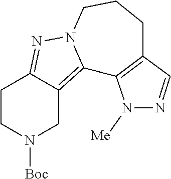 [SMILES: Cn1ncc2c1-c1c3c(nn1CCC2)CCN(C(=O)OC(C)(C)C)C3]

The title compound was isolated by RP HPLC (Condition A) from Intermediate 16. MS (ESI): mass calcd. for C₁₈H₂₅N₅O₂, 343.2; m/z found, 344.2 [M+H]⁺. ¹H NMR (400 MHz, CDCl₃) δ 7.43 (s, 1H), 4.43-4.65 (m, 2H), 4.11-4.25 (m, 2H), 3.91 (s, 3H), 3.76 (br s, 2H), 2.82 (br t, J=5.58 Hz, 2H), 2.71 (t, J=7.47 Hz, 2H), 2.22 (br dd, J=4.96, 6.71 Hz, 2H), 1.48 (s, 8H).

### Intermediate 20: tert-Butyl 5,6,9,10-tetrahydro-4H-isoxazolo[3,4-c]pyrido[4′,3′:3,4]pyrazolo-[1,5-a]azepine-11(12H)-carboxylate

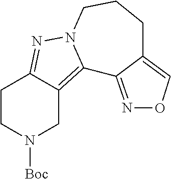 [SMILES: CC(C)(C)OC(=O)N1CCc2nn3c(c2C1)-c1nocc1CCC3]

To a mixture of tert-butyl 10-(hydroxymethylene)-11-oxo-3,4,8,9,10,11-hexahydro-1H-pyrido[4′,3′:3,4]pyrazolo[1,5-a]azepine-2(7H)-carboxylate (80.00 mg, 239.97 umol, Intermediate 15 product from Step A) in Py (5.00 mL) was added NH₂OH.HCl (100.05 mg, 1.44 mmol) in one portion under N₂. The mixture was stirred at 115° C. for 12 h, then concentrated under reduced pressure. The residue was poured into HCl (1 N aq, 10 mL) and stirred for 1 min. The aqueous phase was extracted with ethyl acetate (10 mL×2). The combined organic phases were washed with brine (10 mL×2), dried with anhydrous Na₂SO₄, filtered and the filtrate concentrated under reduced pressure. The residue was purified by prep-TLC (petroleum ether/ethyl acetate=1/2) to afford title compound (48.00 mg, 116.23 umol, 48.44% yield, 80% purity) as a yellow solid. MS(ESI): mass calcd. for C₁₇H₂₂N₄O₃, 330.1; m/z found, 331.1 [M+H]⁺; ¹H NMR (400 MHz, CDCl₃) δ 8.17-8.37 (m, 1H), 4.70 (br s, 2H), 4.38-4.57 (m, 2H), 3.74 (br s, 2H), 3.50 (s, 3H), 2.87-3.03 (m, 2H), 2.65-2.82 (m, 2H), 2.16-2.39 (m, 2H), 1.50 (s, 9H).

### Intermediate 21: tert-Butyl 5,6,9,10-tetrahydro-4H-isoxazolo[5,4-c]pyrido[4′,3′:3,4]pyrazolo[1,5-a]azepine-11(12H)-carboxylate

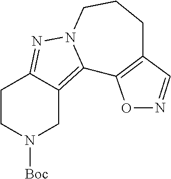 [SMILES: CC(C)(C)OC(=O)N1CCc2nn3c(c2C1)-c1oncc1CCC3]

To a mixture of tert-butyl 10-(hydroxymethylene)-11-oxo-3,4,8,9,10,11-hexahydro-1H-pyrido[4′,3′:3,4]pyrazolo[1,5-a]azepine-2(7H)-carboxylate (70.00 mg, 209.97 umol, Intermediate 15 product from Step A) in MeOH (5.00 mL) was added NH₂OH.HCl (87.55 mg, 1.26 mmol) in one portion at 30° C. under N₂. The mixture was stirred at 30° C. for 12 h. The mixture was poured into water (10 mL) and stirred for 1 min. The aqueous phase was extracted with ethyl acetate (10 mL×2). The combined organic phases were washed with brine (10 mL×2), dried with anhydrous Na₂SO₄, filtered and concentrated under reduced pressure. The residue was purified by prep-TLC (petroleum ether/ethyl acetate=1/2) to afford the title compound (40.00 mg, 106.54 umol, 50.74% yield, 88% purity) as yellow oil. MS (ESI): mass calcd. for C₁₇H₂₂N₄O₃, 330.1; m/z found, 331.1 [M+H]⁺. ¹H NMR (400 MHz, CDCl₃) δ 8.15 (s, 1H), 4.78 (br s, 2H), 4.40-4.57 (m, 2H), 3.74 (br s, 2H), 2.86 (t, J=5.96 Hz, 2H), 2.77 (br s, 2H), 2.19-2.31 (m, 2H), 1.50 (s, 9H).

### Intermediate 22: tert-Butyl 11-oxo-3,4,8,9,10,11-hexahydro-1H-cyclohepta[3,4]pyrazolo[1,5-a]pyrazine-2(7H)-carboxylate

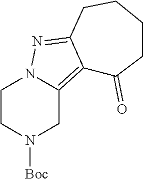 [SMILES: CC(C)(C)OC(=O)N1CCn2nc3c(c2C1)C(=O)CCCC3]

Step A. Diethyl 1-(2-((tert-butoxycarbonyl)amino)ethyl)-1H-pyrazole-3,5-dicarboxylate. To a solution of diethyl 1H-pyrazole-3,5-dicarboxylate (45 g, 212.06 mmol) and Cs₂CO₃ (82.91 g, 254.47 mmol) in DMF (1000 mL) was added tert-butyl N-(2-bromoethyl)carbamate (50.85 g, 226.91 mmol). The mixture was stirred at 15° C. for 16 h under N₂ atmosphere. The reaction mixture was diluted with water (500 mL) and extracted with EtOAc (700 mL×3). The combined organic layers were washed with brine (1000 mL×3), dried over Na₂SO₄, filtered and the filtrate concentrated under reduced pressure to give the title compound (67 g, crude) as a white solid, which was used directly for the next step. ¹H NMR (400 MHz, CDCl₃) δ 7.35 (s, 1H), 4.82-4.74 (m, 3H), 4.42-4.33 (m, 4H), 3.63-3.62 (m, 2H), 1.46-1.38 (m, 15H).

Step B. Ethyl 4-oxo-4,5,6,7-tetrahydropyrazolo[1,5-a]pyrazine-2-carboxylate. To a solution of diethyl 1-[2-(tert-butoxycarbonylamino)ethyl]pyrazole-3,5-dicarboxylate (67 g, 188.53 mmol) in MeOH (100 mL) was added HCl/MeOH (4 M, 100 mL). The mixture was stirred at 15° C. for 16 h. The reaction mixture was concentrated under reduced pressure to give crude product (54.9 g crude, HCl salt) as a white solid. To the resulting solid was added dioxane (560 mL), following by a solution of Na₂CO₃ (39.89 g, 376.36 mmol) in water (560 mL). The mixture was stirred at 15° C. for 16 h. The reaction mixture was extracted with EtOAc (500 mL×2), following by DCM/MeOH=20/1 (500 mL×2). The combined organic layers were dried over Na₂SO₄, filtered and the filtrate concentrated under reduced pressure. The residue was triturated in a mixture of petroleum ether/EtOAc (v/v=10/1, 150 mL) and then filtered. The collected solid was dried to give title compound (34 g, containing ˜60% mol methyl ester) as a white solid.

Step C. tert-Butyl 2-(hydroxymethyl)-6,7-dihydropyrazolo[1,5-a]pyrazine-5(4H)-carboxylate. To a solution of tert-butyl 2-(hydroxymethyl)-6,7-dihydropyrazolo[1,5-a]-pyrazine-5(4H)-carboxylate (32.00 g, containing ˜60% mol methyl ester) in THE (640 mL) was added LAH (6.6 g, 173.91 mmol) at −30° C. under a N₂ atmosphere, then the mixture was heated to 75° C. for 16 h. LAH (6.6 g, 173.89 mmol) was added to the mixture at −30° C. The reaction mixture was heated to 75° C. for 16 h. The reaction mixture was quenched by addition of saturated aqueous potassium sodium tartrate tetrahydrate (30 mL) and stirred for 1 h and filtered. To the filtrate was added Boc₂O (50.12 g, 229.67 mmol, 52.76 mL) and stirred at 15° C. for 16 h. The reaction mixture was diluted with water (600 mL) and extracted with EtOAc (300 mL×2). The combined organic layers were washed with brine (400 mL), dried over Na₂SO₄, filtered and the filtrate concentrated under reduced pressure. The residue was purified by column chromatography to give title product (33 g, 130.28 mmol, 85.09% yield) as a white solid. ¹H NMR (400 MHz, CDCl₃) δ 6.04 (s, 1H), 4.62-4.61 (m, 4H), 4.13-4.10 (m, 2H), 3.86-3.84 (m, 2H), 1.47 (s, 9H).

Step D. tert-Butyl 2-(hydroxymethyl)-3-iodo-6,7-dihydropyrazolo[1,5-a]pyrazine-5(4H)-carboxylate. A solution of tert-butyl 2-(hydroxymethyl)-6,7-dihydro-4H-pyrazolo[1,5-a]pyrazine-5-carboxylate (23 g, 90.80 mmol) in MeCN (300 mL) was added NIS (30.64 g, 136.20 mmol) slowly, and the mixture was stirred at 15° C. for 16 h under a N₂ atmosphere. The mixture was diluted with water (400 mL) and extracted with EtOAc (400 mL). The organic phases were washed with saturated Na₂S₂O₃ (400 mL), dried over Na₂SO₄, filtered and the filtrate concentrated under reduced pressure. The residue was rinsed with petroleum ether/EtOAc=20/1 (300 mL) and stirred for 0.5 h. The mixture was filtered. The collected solid was dried under reduced pressure to give title compound (29.5 g, 77.80 mmol, 85.68% yield) as a yellow solid. ¹H NMR (400 MHz, CDCl₃) δ 4.61 (s, 2H), 4.48 (s, 2H), 4.14 (m, 2H), 3.86 (m, 2H).

Step E. tert-Butyl 2-formyl-3-iodo-6,7-dihydropyrazolo[1,5-a]pyrazine-5(4H)-carboxylate. To a solution of tert-butyl 2-(hydroxymethyl)-3-iodo-6,7-dihydro-4H-pyrazolo[1,5-a]pyrazine-5-carboxylate (9 g, 23.73 mmol) in DCM (180 mL) was added Dess-Martin (15.10 g, 35.60 mmol, 11.02 mL) and the mixture was stirred at 15° C. for 2 h. The mixture was filtered, and the filtrate was diluted with DCM (300 mL) and washed with brine (300 mL). The organic phases were dried over Na₂SO₄, filtered and the filtrate concentrated under reduced pressure. The residue was purified by silica gel column chromatography to give title compound (7.5 g, 19.88 mmol, 83.78% yield) as a yellow solid.

Step F. tert-Butyl 3-iodo-2-vinyl-6,7-dihydropyrazolo[1,5-a]pyrazine-5(4H)-carboxylate. To a solution of methyl(triphenyl)phosphonium bromide (9.23 g, 25.85 mmol) in THE (50 mL) was added NaHMDS (1 M, 25.85 mL) at −10° C. under a N₂ atmosphere, followed by a solution of tert-butyl 2-formyl-3-iodo-6,7-dihydro-4H-pyrazolo[1,5-a]pyrazine-5-carboxylate (7.5 g, 19.88 mmol) in THE (30 mL) after 0.5 h and the mixture was stirred at 15° C. for 2 h. The mixture was quenched with brine (120 mL) and extracted with EtOAc (120 mL). The organic phases were dried over Na₂SO₄, filtered and the filtrate concentrated under reduced pressure. The residue was purified by silica gel column chromatography to give pure title compound (2.8 g, 7.46 mmol) as a colorless oil.

Step G. tert-Butyl 3-(1-hydroxypent-4-en-1-yl)-2-vinyl-6,7-dihydropyrazolo[1,5-a]pyrazine-5(4H)-carboxylate. To a solution of tert-butyl 3-iodo-2-vinyl-6,7-dihydro-4H-pyrazolo[1,5-a]pyrazine-5-carboxylate (1.8 g, 4.80 mmol) in THE (30 mL) was added i-PrMgCl (2 M, 3.60 mL) at −10° C. under a N₂ atmosphere. The mixture was stirred at 10° C. for 1 h, then a solution of pent-4-enal (605.31 mg, 7.20 mmol) in THE (3 mL) was added. The reaction mixture was stirred at 15° C. for 1.5 h. The mixture was quenched with saturated NH₄Cl (100 mL) and extracted with EtOAc (100 mL). The organic phases were dried over Na₂SO₄, filtered and the filtrate concentrated under reduced pressure. The residue was purified by silica gel column chromatography to give title compound (1.0 g, 3.00 mmol, 62.52% yield) as a colorless oil.

Step H. tert-Butyl 11-hydroxy-3,4,10,11-tetrahydro-1H-cyclohepta[3,4]pyrazolo[1,5-a]pyrazine-2(9H)-carboxylate. To a solution of tert-butyl 3-(1-hydroxypent-4-enyl)-2-vinyl-6,7-dihydro-4H-pyrazolo[1,5-a]pyrazine-5-carboxylate (1.3 g, 3.90 mmol) in DCM (800 mL) was added [1,3-bis(2,4,6-trimethylphenyl)imidazolidin-2-ylidene]-dichloro-[(2-isopropoxyphenyl)methylene]ruthenium (244.32 mg, 389.89 umol) under a N₂ atmosphere, and the mixture was stirred at 40° C. for 16 h. The mixture was concentrated under reduced pressure. The residue was purified by silica gel column chromatography to give title compound (0.79 g, 2.59 mmol, 66.35% yield) as a brown solid. MS (ESI): mass calcd. for C₁₆H₂₃N₃O₃ 305.2; m/z found, 306.1 [M+H]⁺.

Step I. tert-Butyl 11-oxo-3,4,10,11-tetrahydro-1H-cyclohepta[3,4]pyrazolo[1,5-a]pyrazine-2(9H)-carboxylate. A mixture of tert-butyl 11-hydroxy-1,3,4,9,10,11-hexahydrocyclohepta-[2,3]pyrazolo[2,4-a]pyrazine-2-carboxylate (570 mg, 1.87 mmol), NMO (874.65 mg, 7.47 mmol, 787.97 uL) and TPAP (131.19 mg, 373.32 umol) in MeCN (10 mL) was degassed and purged with N₂ (3×), and then the mixture was stirred at 15° C. for 1.5 h under a N₂ atmosphere. The mixture was poured into ice-water (50 mL) and stirred for 1 min. The aqueous phase was extracted with ethyl acetate (30 mL×2). The combined organic phases were washed with brine (60 mL), dried with anhydrous Na₂SO₄, filtered and the filtrate concentrated under reduced pressure. The residue was purified by column chromatography to give title compound (405 mg, 1.34 mmol, 71.52% yield) as a black brown solid. MS (ESI): mass calcd. for C₁₆H₂₁N₃O₃ 303.2; m/z found, 304.1 [M+H]⁺.

Step J. tert-Butyl 11-oxo-3,4,8,9,10,11-hexahydro-1H-cyclohepta[3,4]pyrazolo[1,5-a]pyrazine-2(7H)-carboxylate. To a solution of tert-butyl 11-oxo-3,4,9,10-tetrahydro-1H-cyclohepta[2,3]pyrazolo[2,4-a] pyrazine-2-carboxylate (0.405 g, 1.34 mmol) in EtOH (30 mL)/MeOH (3 mL) was added Pd/C (0.08 g, 1.34 mmol, 10% purity) and the mixture was stirred at 15° C. under H₂ (15 Psi) atmosphere for 1 h. The mixture was filtered, the filtrate was concentrated under reduced pressure to give title compound (0.39 g, 1.28 mmol, 95.66% yield) as a brown solid, which was used directly for the next step. ¹H NMR (400 MHz, CDCl₃) δ 4.77 (s, 2H), 4.05-4.03 (m, 2H), 3.81-3.79 (m, 2H), 2.89-2.86 (m, 2H), 2.62-2.59 (m, 2H), 1.89-1.82 (m, 4H), 1.44 (s, 9H).

### Intermediate 23: tert-Butyl 5,6,9,10-tetrahydro-4H-isoxazolo[5″,4″:3′,4′]cyclohepta[1′,2′:3,4]-pyrazolo[1,5-a]pyrazine-11(12H)-carboxylate

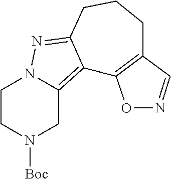 [SMILES: CC(C)(C)OC(=O)N1CCn2nc3c(c2C1)-c1oncc1CCC3]

Step A. tert-Butyl 10-((dimethylamino)methylene)-11-oxo-3,4,8,9,10,11-hexahydro-1H-cyclohepta[3,4]pyrazolo[1,5-a]pyrazine-2(7H)-carboxylate. A solution of tert-butyl 11-oxo-3,4,7,8,9,10-hexahydro-1H-cyclohepta[2,3]pyrazolo[2,4-a]pyrazine-2-carboxylate (0.08 g, 261.98 umol) in DMF-DMA (3.59 g, 30.11 mmol, 4 mL) was heated to 115° C. for 56 h. The mixture was concentrated under reduced pressure. The residue was diluted with EtOAc (30 mL) and washed with brine (30 mL). The organic phases were dried over Na₂SO₄, filtered and the filtrate concentrated under reduced pressure to give title compound (0.09 g, crude) as a yellow solid, which was used directly for the next step.

### Step B. tert-Butyl 5,6,9,10-tetrahydro-4H-isoxazolo[5″,4″:3′,4′]cyclohepta[1′,2′:3,4]pyrazolo-[1,5-a]pyrazine-11(12H)-carboxylate

A mixture of tert-butyl 10-((dimethylamino)methylene)-11-oxo-3,4,8,9,10,11-hexahydro-1H-cyclohepta[3,4]pyrazolo[1,5-a]pyrazine-2(7H)-carboxylate (0.09 g, 249.69 umol) and hydroxylamine hydrochloride (104.11 mg, 1.50 mmol) in MeOH (3 mL) was stirred at 20° C. for 16 h. The mixture was diluted with EtOAc (40 mL) and washed with brine (40 mL). The organic phases were dried over Na₂SO₄, filtered, and the filtrate concentrated under reduced pressure. The residue was purified by prep-TLC (Petroleum ether/EtOAc) to give title compound (0.051 g, 140.47 umol, 56.26% yield, 91% purity) as a colorless oil. MS (ESI): mass calcd. for C₁₇H₂₂N₄O₃ 330.2; m/z found, 331.1 [M+H]⁺.

¹H NMR (400 MHz, CDCl₃) δ 8.03 (s, 1H), 4.97 (s, 2H), 4.16-4.13 (m, 2H), 3.93-3.90 (m, 2H), 3.02-2.99 (m, 2H), 2.79-2.76 (m, 2H), 2.05-2.00 (m, 2H), 1.51 (s, 9H).

### Intermediate 24: tert-Butyl 5,6,9,10-tetrahydro-4H-isoxazolo [3″,4″:3′,4′] cyclohepta [1′,2′:3,4]pyrazolo[1,5-a]pyrazine-11(12H)-carboxylate

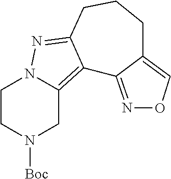 [SMILES: CC(C)(C)OC(=O)N1CCn2nc3c(c2C1)-c1nocc1CCC3]

Step A. tert-Butyl 10-((dimethylamino)methylene)-11-oxo-3,4,8,9,10,11-hexahydro-1H-cyclohepta[3,4]pyrazolo[1,5-a]pyrazine-2(7H)-carboxylate. A solution of tert-butyl 11-oxo-3,4,7,8,9,10-hexahydro-1H-cyclohepta[2,3]pyrazolo[2,4-a]pyrazine-2-carboxylate (0.34 g, 1.11 mmol) and TDAM (1.29 g, 8.91 mmol, 1.54 mL) in toluene (15 mL) was heated to 115° C. for 16 h. TDAM (646.87 mg, 4.45 mmol) was added and the mixture was heated to 115° C. for another 16 h. Additional TDAM (323.43 mg, 2.23 mmol) was added and the mixture was heated to 115° C. for another 16 h. At that time, the mixture was diluted with EtOAc (60 mL) and washed with brine (50 mL×3). The organic phases were dried over Na₂SO₄, filtered and the filtrate concentrated under reduced pressure to give title compound (0.385 g, crude) as a yellow solid, which was used directly for the next step.

### Step B. tert-Butyl 5,6,9,10-tetrahydro-4H-isoxazolo[3″,4″:3′,4′]cyclohepta[1′,2′:3,4]pyrazolo-[1,5-a]pyrazine-11(12H)-carboxylate

A mixture of tert-butyl 10-((dimethylamino)methylene)-11-oxo-3,4,8,9,10,11-hexahydro-1H-cyclohepta[3,4]pyrazolo[1,5-a]pyrazine-2(7H)-carboxylate (0.235 g, 651.96 umol) and hydroxylamine hydrochloride (271.83 mg, 3.91 mmol) in pyridine (12 mL) was stirred at 115° C. for 24 h. The mixture was concentrated to give a yellow residue, which was diluted with EtOAc (50 mL) and washed with HCl (1 M aq, 50 mL). The organic phases were dried over Na₂SO₄, filtered and the filtrate concentrated under reduced pressure. The residue was purified by prep-HPLC (Condition A) to give regioisomer compound tert-butyl 5,6,9,10-tetrahydro-4H-isoxazolo[5″,4″:3′,4′]cyclohepta[1′,2′:3,4]pyrazolo[1,5-a]pyrazine-11(12H)-carboxylate (intermediate 2, 0.07 g, 211.88 umol, 32.50% yield) as a colorless oil, and title compound (0.037 g, 111.99 umol, 17.18% yield) as a colorless oil. ¹H NMR (400 MHz, CDCl₃) δ 8.11 (s, 1H), 4.90 (s, 2H), 4.18-4.15 (m, 2H), 3.93-3.90 (m, 2H), 3.07-3.04 (m, 2H), 2.85-2.83 (m, 2H), 2.01-1.98 (m, 2H), 1.51 (s, 9H).

### Example 1: N-(3-Cyano-4-fluorophenyl)-5-methylene-5,6,9,10-tetrahydro-4H-isoxazolo[3,4-c]pyrido[4′,3′:3,4]pyrazolo[1,5-a]azepine-11(12H)-carboxamide

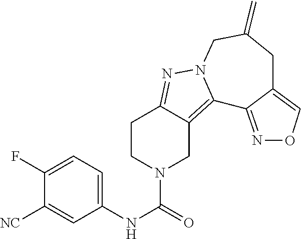 [SMILES: C=C1Cc2conc2-c2c3c(nn2C1)CCN(C(=O)Nc1ccc(F)c(C#N)c1)C3]

### Step A. 5-Methylene-5,6,9,10,11,12-hexahydro-4H-isoxazolo[3,4-c]pyrido[4′,3′:3,4]-pyrazolo[1,5-a]azepine

To a solution of tert-butyl 5-methylene-5,6,9,10-tetrahydro-4H-isoxazolo[3,4-c]pyrido-[4′,3′:3,4]pyrazolo[1,5-a]azepine-11(12H)-carboxylate (Intermediate 1, 0.06 g, 175.24 umol) in DCM (5 mL) was added TFA (770.00 mg, 6.75 mmol, 0.5 mL). The mixture was stirred at 20° C. for 1 h. The reaction mixture was concentrated under reduced pressure to give the title compound (63 mg, crude, TFA salt) as a yellow oil. MS (ESI): mass calcd. for C₁₃H₁₄N₄O, 242.17; m/z found, 243.1 [M+H]⁺.

### Step B. N-(3-Cyano-4-fluorophenyl)-5-methylene-5,6,9,10-tetrahydro-4H-isoxazolo[3,4-c]pyrido[4′,3′:3,4]pyrazolo[1,5-a]azepine-11(12H)-carboxamide

To a solution of 5-methylene-5,6,9,10,11,12-hexahydro-4H-isoxazolo[3,4-c]pyrido[4′,3′:3,4]-pyrazolo[1,5-a]azepine (63 mg, 182.43 umol, TFA salt) and phenyl N-(3-cyano-4-fluoro-phenyl)carbamate (44 mg, 154.55 umol) in DCM (5 mL) was added TEA (184.60 mg, 1.82 mmol, 253.92 uL). The mixture was stirred at 25° C. for 12 h. The reaction mixture was concentrated under reduced pressure. The residue was purified by RP HPLC (Condition A) to give the title compound (40.58 mg, 99.34 umol, 54.46% yield, 99% purity) as a white solid. MS (ESI): mass calcd. for C₂₁H₁₇FN₆O₂, 404.1; m/z found, 405.1 [M+H]⁺; ¹H NMR (400 MHz, CDCl₃) δ=8.36 (s, 1H), 7.77 (dd, J=2.8, 5.6 Hz, 1H), 7.65-7.61 (m, 1H), 7.13 (t, J=8.8 Hz, 1H), 6.82 (s, 1H), 5.39 (s, 1H), 5.31 (s, 1H), 4.97 (s, 2H), 4.73 (s, 2H), 3.90 (t, J=5.6 Hz, 2H), 3.66 (s, 2H), 2.89 (t, J=5.6 Hz, 2H).

### Example 2: N-(4-Fluoro-3-(trifluoromethyl)phenyl)-5-methylene-5,6,9,10-tetrahydro-4H-isoxazolo[3,4-c]pyrido[4′,3′:3,4]pyrazolo[1,5-a]azepine-11(12H)-carboxamide

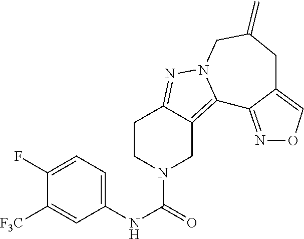 [SMILES: C=C1Cc2conc2-c2c3c(nn2C1)CCN(C(=O)Nc1ccc(F)c(C(F)(F)F)c1)C3]

The title compound was prepared in a manner analogous to Example 1, using phenyl (4-fluoro-3-(trifluoromethyl)phenyl)carbamate instead of phenyl (3-cyano-4-fluorophenyl)-carbamate in Step B. MS (ESI): mass calcd. for C₂₁H₁₇F₄N₅O₂, 447.13; m/z found, 448.1 [M+H]⁺. ¹H NMR (400 MHz, CDCl₃) δ=8.36 (s, 1H), 7.68 (dd, J=2.4, 6.0 Hz, 1H), 7.64-7.59 (m, 1H), 7.13 (t, J=9.2 Hz, 1H), 6.72 (s, 1H), 5.39 (s, 1H), 5.31 (s, 1H), 4.98 (s, 2H), 4.73 (s, 2H), 3.91 (t, J=5.6 Hz, 2H), 3.66 (s, 2H), 2.89 (t, J=5.6 Hz, 2H).

### Example 3: N-(3-Cyano-4-fluorophenyl)-5-(hydroxymethyl)-5,6,9,10-tetrahydro-4H-isoxazolo[3,4-c]pyrido[4′,3′:3,4]pyrazolo[1,5-a]azepine-11(12H)-carboxamide

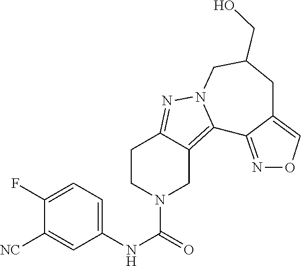 [SMILES: N#Cc1cc(NC(=O)N2CCc3nn4c(c3C2)-c2nocc2CC(CO)C4)ccc1F]

The title compound was prepared in a manner analogous to Example 1, using tert-butyl 5-(hydroxymethyl)-5,6,9,10-tetrahydro-4H-isoxazolo[3,4-c]pyrido[4′,3′:3,4]pyrazolo[1,5-a]azepine-11(12H)-carboxylate (Intermediate 2) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₁H₁₉FN₆O₃, 422.15; m/z found, 423.1 [M+H]⁺. ¹H NMR (400 MHz, CDCl₃) δ=8.35 (s, 1H), 7.78 (dd, J=2.8, 5.6 Hz, 1H), 7.67-7.63 (m, 1H), 7.14 (t, J=8.8 Hz, 1H), 6.90 (s, 1H), 4.75-4.68 (m, 3H), 4.46-4.37 (m, 1H), 3.93-3.87 (m, 2H), 3.74-3.66 (m, 2H), 3.14-3.08 (m, 1H), 2.90-2.80 (m, 3H), 2.45 (d, J=6.4 Hz, 1H).

### Example 4: N-(4-Fluoro-3-(trifluoromethyl)phenyl)-5-(hydroxymethyl)-5,6,9,10-tetrahydro-4H-isoxazolo[3,4-c]pyrido[4′,3′:3,4]pyrazolo[1,5-a]azepine-11(12H)-carboxamide

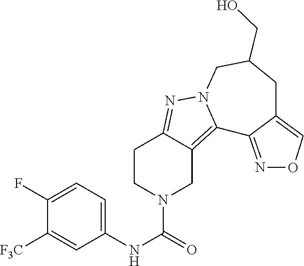 [SMILES: O=C(Nc1ccc(F)c(C(F)(F)F)c1)N1CCc2nn3c(c2C1)-c1nocc1CC(CO)C3]

The title compound was prepared in a manner analogous to Example 1, using tert-butyl 5-(hydroxymethyl)-5,6,9,10-tetrahydro-4H-isoxazolo[3,4-c]pyrido[4′,3′:3,4]pyrazolo[1,5-a]azepine-11(12H)-carboxylate (Intermediate 2) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A, and using phenyl (4-fluoro-3-(trifluoromethyl)phenyl)carbamate instead of phenyl (3-cyano-4-fluorophenyl)carbamate in Step B. MS (ESI): mass calcd. for C₂₁H₁₉F₄N₅O₃, 465.1; m/z found, 466.1 [M+H]⁺. ¹H NMR (400 MHz, CDCl₃) δ=8.34 (s, 1H), 7.69 (dd, J=2.4, 6.0 Hz, 1H), 7.65-7.60 (m, 1H), 7.13 (t, J=9.6 Hz, 1H), 6.77 (s, 1H), 4.76-4.67 (m, 3H), 4.46-4.37 (m, 1H), 3.93-3.87 (m, 2H), 3.75-3.65 (m, 2H), 3.15-3.07 (m, 1H), 2.90-2.78 (m, 3H), 2.50-2.40 (m, 1H).

### Example 5: (5S*)—N-(3-Cyano-4-fluorophenyl)-5-((2,2-difluoroethoxy)methyl)-5,6,9,10-tetrahydro-4H-isoxazolo[3,4-c]pyrido[4′,3′:3,4]pyrazolo[1,5-a]azepine-11(12H)-carboxamide

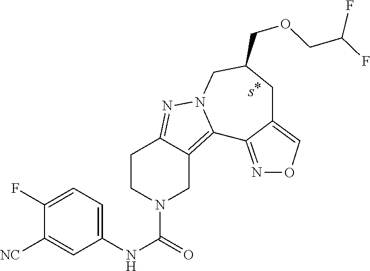 [SMILES: N#Cc1cc(NC(=O)N2CCc3nn4c(c3C2)-c2nocc2C[C@H](COCC(F)F)C4)ccc1F]

The title compound was prepared in a manner analogous to Example 1, using (5S*)-tert-butyl 5-((2,2-difluoroethoxy)methyl)-5,6,9,10-tetrahydro-4H-isoxazolo[3,4-c]pyrido[4′,3′:3,4]-pyrazolo[1,5-a]azepine-11(12H)-carboxylate (Intermediate 3) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₃H₂₁F₃N₆O₃, 486.2; m/z found, 487.1 [M+H]⁺. ¹H NMR (400 MHz, CDCl₃) δ=8.35 (s, 1H), 7.78 (dd, J=2.8, 5.6 Hz, 1H), 7.66-7.62 (m, 1H), 7.14 (t, J=8.8 Hz, 1H), 6.80 (s, 1H), 6.03-5.70 (m, 1H), 4.79-4.72 (m, 2H), 4.70-4.66 (m, 1H), 4.40-4.34 (m, 1H), 3.93-3.88 (m, 2H), 3.73-3.63 (m, 2H), 3.60 (d, J=6.4 Hz, 2H), 3.10-3.05 (m, 1H), 2.91-2.84 (m, 3H), 2.59-2.48 (m, 1H).

### Example 6: (5S*)-5-((2,2-Difluoroethoxy)methyl)-N-(4-fluoro-3-(trifluoromethyl)phenyl)-5,6,9,10-tetrahydro-4H-isoxazolo[3,4-c]pyrido[4′,3′:3,4]pyrazolo[1,5-a]azepine-11(12H)-carboxamide

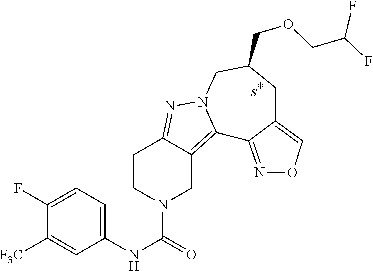 [SMILES: O=C(Nc1ccc(F)c(C(F)(F)F)c1)N1CCc2nn3c(c2C1)-c1nocc1C[C@H](COCC(F)F)C3]

The title compound was prepared in a manner analogous to Example 1, using (5S*)-tert-butyl 5-((2,2-difluoroethoxy)methyl)-5,6,9,10-tetrahydro-4H-isoxazolo[3,4-c]pyrido-[4′,3′:3,4]pyrazolo[1,5-a]azepine-11(12H)-carboxylate (Intermediate 3) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A, and using phenyl (4-fluoro-3-(trifluoro-methyl)phenyl)carbamate instead of phenyl (3-cyano-4-fluorophenyl)carbamate in Step B. MS (ESI): mass calcd. for C₂₃H₂₁F₆N₅O₃, 529.2; m/z found, 530.1 [M+H]⁺. ¹H NMR (400 MHz, CDCl₃) δ=8.35 (s, 1H), 7.69 (dd, J=2.8, 6.0 Hz, 1H), 7.66-7.59 (m, 1H), 7.14 (t, J=9.2 Hz, 1H), 6.73 (s, 1H), 6.03-5.71 (m, 1H), 4.80-4.73 (m, 2H), 4.70-4.66 (m, 1H), 4.40-4.34 (m, 1H), 3.96-3.87 (m, 2H), 3.71-3.63 (m, 2H), 3.60 (d, J=6.4 Hz, 2H), 3.10-3.05 (m, 1H), 2.91-2.84 (m, 3H), 2.60-2.48 (m, 1H).

### Example 7: (5R*)—N-(3-Cyano-4-fluorophenyl)-5-((2,2-difluoroethoxy)methyl)-5,6,9,10-tetrahydro-4H-isoxazolo[3,4-c]pyrido[4′,3′:3,4]pyrazolo[1,5-a]azepine-11(12H)-carboxamide

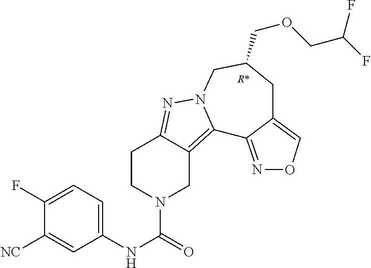 [SMILES: N#Cc1cc(NC(=O)N2CCc3nn4c(c3C2)-c2nocc2C[C@@H](COCC(F)F)C4)ccc1F]

The title compound was prepared in a manner analogous to Example 1, using (5R*)-tert-butyl 5-((2,2-difluoroethoxy)methyl)-5,6,9,10-tetrahydro-4H-isoxazolo[3,4-c]pyrido[4′,3′:3,4]-pyrazolo[1,5-a]azepine-11(12H)-carboxylate (Intermediate 4) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₃H₂₁F₃N₆O₃, 486.2; m/z found, 487.1 [M+H]⁺. ¹H NMR (400 MHz, CDCl₃) δ=8.36 (s, 1H), 7.79 (dd, J=2.8, 5.6 Hz, 1H), 7.68-7.64 (m, 1H), 7.14 (t, J=8.8 Hz, 1H), 6.95 (s, 1H), 6.02-5.71 (m, 1H), 4.76-4.66 (m, 3H), 4.40-4.34 (m, 1H), 3.91 (q, J=5.6 Hz, 2H), 3.71-3.63 (m, 2H), 3.60 (d, J=6.4 Hz, 2H), 3.10-3.06 (m, 1H), 2.92-2.84 (m, 3H), 2.59-2.49 (m, 1H).

### Example 8: (5R*)-5-((2,2-Difluoroethoxy)methyl)-N-(4-fluoro-3-(trifluoromethyl)phenyl)-5,6,9,10-tetrahydro-4H-isoxazolo[3,4-c]pyrido[4′,3′:3,4]pyrazolo[1,5-a]azepine-11(12H)-carboxamide

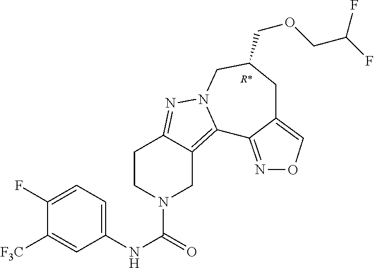 [SMILES: O=C(Nc1ccc(F)c(C(F)(F)F)c1)N1CCc2nn3c(c2C1)-c1nocc1C[C@@H](COCC(F)F)C3]

The title compound was prepared in a manner analogous to Example 1, except using (5R*)-tert-butyl 5-((2,2-difluoroethoxy)methyl)-5,6,9,10-tetrahydro-4H-isoxazolo[3,4-c]pyrido-[4′,3′:3,4]pyrazolo[1,5-a]azepine-11(12H)-carboxylate (Intermediate 4) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A, and using phenyl (4-fluoro-3-(trifluoro-methyl)phenyl)carbamate instead of phenyl (3-cyano-4-fluorophenyl)carbamate in Step B. MS (ESI): mass calcd. for C₂₃H₂₁F₆N₅O₃, 529.2; m/z found, 530.1 [M+H]⁺. ¹H NMR (400 MHz, CDCl₃) δ=8.35 (s, 1H), 7.70 (dd, J=2.8, 6.0 Hz, 1H), 7.66-7.60 (m, 1H), 7.13 (t, J=9.2 Hz, 1H), 6.85 (s, 1H), 6.03-5.70 (m, 1H), 4.77-4.64 (m, 3H), 4.40-4.34 (m, 1H), 3.94-3.88 (m, 2H), 3.73-3.63 (m, 2H), 3.60 (d, J=6.4 Hz, 2H), 3.13-3.05 (m, 1H), 2.92-2.83 (m, 3H), 2.54 (s, 1H).

### Example 9: N-(3-Cyano-4-fluorophenyl)-5-methylene-5,6,9,10-tetrahydro-4H-isoxazolo[5,4-c]pyrido[4′,3′:3,4]pyrazolo[1,5-a]azepine-11(12H)-carboxamide

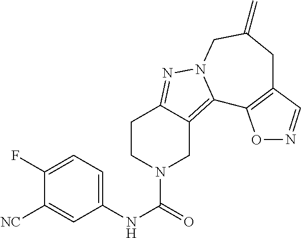 [SMILES: C=C1Cc2cnoc2-c2c3c(nn2C1)CCN(C(=O)Nc1ccc(F)c(C#N)c1)C3]

The title compound was prepared in a manner analogous to Example 1, except using tert-butyl 5-methylene-5,6,9,10-tetrahydro-4H-isoxazolo[5,4-c]pyrido[4′,3′:3,4]pyrazolo[1,5-a]azepine-11(12H)-carboxylate (Intermediate 5) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₁H₁₇FN₆O₂, 404.1; m/z found, 405.1 [M+H]⁺. ¹H NMR (400 MHz, CDCl₃) δ=8.22 (s, 1H), 7.78 (dd, J=2.8, 5.4 Hz, 1H), 7.64-7.60 (m, 1H), 7.15 (t, J=8.8 Hz, 1H), 6.68 (s, 1H), 5.42 (s, 1H), 5.36 (s, 1H), 4.90 (s, 2H), 4.81 (s, 2H), 3.88 (t, J=5.6 Hz, 2H), 3.60 (s, 2H), 2.88 (t, J=5.6 Hz, 2H).

### Example 10: N-(4-Fluoro-3-(trifluoromethyl)phenyl)-5-methylene-5,6,9,10-tetrahydro-4H-isoxazolo[5,4-c]pyrido[4′,3′:3,4]pyrazolo[1,5-a]azepine-11(12H)-carboxamide

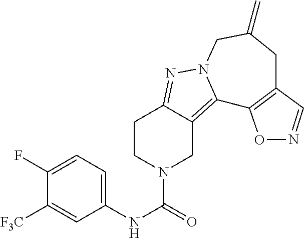 [SMILES: C=C1Cc2cnoc2-c2c3c(nn2C1)CCN(C(=O)Nc1ccc(F)c(C(F)(F)F)c1)C3]

The title compound was prepared in a manner analogous to Example 1, using tert-butyl 5-methylene-5,6,9,10-tetrahydro-4H-isoxazolo[5,4-c]pyrido[4′,3′:3,4]pyrazolo[1,5-a]azepine-11(12H)-carboxylate (Intermediate 5) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (4-fluoro-3-(trifluoromethyl)phenyl)carbamate instead of phenyl (3-cyano-4-fluorophenyl)carbamate in Step B. MS (ESI): mass calcd. for C₂₁H₁₇F₄N₅O₂, 447.13; m/z found, 448.1 [M+H]⁺. ¹H NMR (400 MHz, CDCl₃) δ=8.22 (s, 1H), 7.69 (dd, J=2.8, 6.0 Hz, 1H), 7.64-7.59 (m, 1H), 7.14 (t, J=9.6 Hz, 1H), 6.63 (s, 1H), 5.42 (s, 1H), 5.36 (s, 1H), 4.90 (s, 2H), 4.82 (s, 2H), 3.89 (t, J=5.6 Hz, 2H), 3.60 (s, 2H), 2.88 (t, J=5.6 Hz, 2H).

### Example 11: N-(3-Cyano-4-fluorophenyl)-5-hydroxy-5,6,9,10-tetrahydro-4H-isoxazolo[3,4-c]pyrido[4′,3′:3,4]pyrazolo[1,5-a]azepine-11(12H)-carboxamide

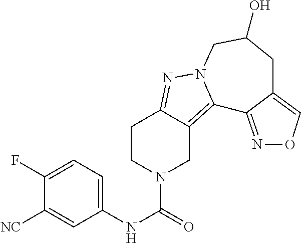 [SMILES: N#Cc1cc(NC(=O)N2CCc3nn4c(c3C2)-c2nocc2CC(O)C4)ccc1F]

The title compound was prepared in a manner analogous to Example 1, using tert-butyl 5-hydroxy-5,6,9,10-tetrahydro-4H-isoxazolo[3,4-c]pyrido[4′,3′:3,4]pyrazolo[1,5-a]azepine-11(12H)-carboxylate (Intermediate 6) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₀H₁₇FN₆O₃, 408.1; m/z found, 409 [M+H]⁺. ¹H NMR (400 MHz, CD₃OD) δ=8.63 (s, 1H), 7.83 (dd, J=2.8, 5.6 Hz, 1H), 7.72 (ddd, J=2.8, 4.8, 9.2 Hz, 1H), 7.28 (t, J=9.2 Hz, 1H), 4.81 (s, 2H), 4.66-4.56 (m, 2H), 4.40 (q, J=5.2 Hz, 1H), 3.88 (t, J=5.6 Hz, 2H), 3.14 (d, J=5.2 Hz, 2H), 2.86 (t, J=5.7 Hz, 2H).

### Example 12: N-(4-Fluoro-3-(trifluoromethyl)phenyl)-5-methyl-5,6,9,10-tetrahydro-4H-isoxazolo[3,4-c]pyrido[4′,3′:3,4]pyrazolo[1,5-a]azepine-11(12H)-carboxamide

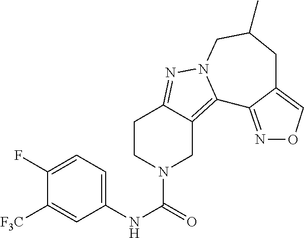 [SMILES: CC1Cc2conc2-c2c3c(nn2C1)CCN(C(=O)Nc1ccc(F)c(C(F)(F)F)c1)C3]

To a solution of N-(4-fluoro-3-(trifluoromethyl)phenyl)-5-methylene-5,6,9,10-tetrahydro-4H-isoxazolo[3,4-c]pyrido[4′,3′:3,4]pyrazolo[1,5-a]azepine-11(12H)-carboxamide (45 mg, 98.57 umol) in MeOH (2 mL) was added Pd—C(10%, 4 mg) under N₂. The suspension was degassed under reduced pressure and purged with H₂ several times. The mixture was stirred under H₂ (15 psi) at 25° C. for 10 min. The reaction mixture was filtered and concentrated in vacuo. The residue was purified by RP HPLC (Condition A) to give N-(4-fluoro-3-(trifluoromethyl)phenyl)-5-methyl-5,6,9,10-tetrahydro-4H-isoxazolo[3,4-c]pyrido[4′,3′:3,4]-pyrazolo[1,5-a]azepine-11(12H)-carboxamide (24.11 mg, 53.11 umol, 53.88% yield, 99% purity) as a white solid. MS (ESI): mass calcd. for C₂₁H₁₉F₄N₅O₂, 449.2; m/z found, 450.2 [M+H]⁺. ¹H NMR (400 MHz, CDCl₃) δ=8.31 (s, 1H), 7.70 (dd, J=2.8, 6.0 Hz, 1H), 7.65-7.60 (m, 1H), 7.14 (t, J=9.6 Hz, 1H), 6.75 (s, 1H), 4.75 (d, J=3.2 Hz, 2H), 4.55-4.52 (m, 1H), 4.32-4.27 (m, 1H), 3.94-3.88 (m, 2H), 3.04-3.00 (m, 1H), 2.88 (t, J=5.6 Hz, 2H), 2.76-2.69 (m, 1H), 2.44 (d, J=6.8 Hz, 1H), 1.16 (d, J=7.2 Hz, 3H).

### Example 13: N-(3-Cyano-4-fluorophenyl)-5-methyl-5,6,9,10-tetrahydro-4H-isoxazolo[3,4-c]pyrido[4′,3′:3,4]pyrazolo[1,5-a]azepine-11(12H)-carboxamide

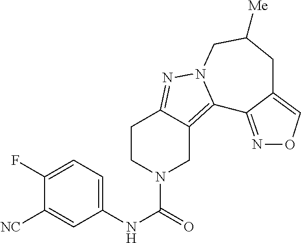 [SMILES: CC1Cc2conc2-c2c3c(nn2C1)CCN(C(=O)Nc1ccc(F)c(C#N)c1)C3]

The title compound was prepared in a manner analogous to Example 12, using N-(3-cyano-4-fluorophenyl)-5-methylene-5,6,9,10-tetrahydro-4H-isoxazolo[3,4-c]pyrido[4′,3′:3,4]-pyrazolo[1,5-a]azepine-11(12H)-carboxamide (Example 1) instead of N-(4-fluoro-3-(trifluoromethyl)phenyl)-5-methylene-5,6,9,10-tetrahydro-4H-isoxazolo[3,4-c]pyrido-[4′,3′:3,4]pyrazolo[1,5-a]azepine-11(12H)-carboxamide. MS (ESI): mass calcd. for C₂₁H₁₉FN₆O₂, 406.2; m/z found, 407.1 [M+H]⁺. ¹H NMR (400 MHz, CDCl₃) δ=8.32 (s, 1H), 7.79 (dd, J=2.8, 5.6 Hz, 1H), 7.68-7.61 (m, 1H), 7.14 (t, J=8.8 Hz, 1H), 6.78 (s, 1H), 4.74 (d, J=3.2 Hz, 2H), 4.55-4.52 (m, 1H), 4.33-4.27 (m, 1H), 3.94-3.88 (m, 2H), 3.04-3.00 (m, 1H), 2.88 (t, J=5.6 Hz, 2H), 2.76-2.69 (m, 1H), 2.44 (d, J=5.6 Hz, 1H), 1.16 (d, J=7.2 Hz, 3H).

### Example 14: (10R)—N-(3-Cyano-4-fluorophenyl)-10-methyl-5,6,9,10-tetrahydro-4H-isoxazolo[5,4-c]pyrido[4′,3′:3,4]pyrazolo[1,5-a]azepine-11(12H)-carboxamide

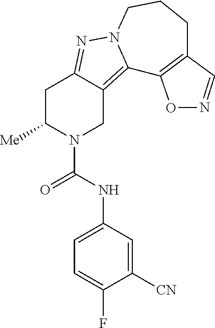 [SMILES: C[C@@H]1Cc2nn3c(c2CN1C(=O)Nc1ccc(F)c(C#N)c1)-c1oncc1CCC3]

The title compound was prepared in a manner analogous to Example 1, using (10R)-tert-butyl 10-methyl-5,6,9,10-tetrahydro-4H-isoxazolo[5,4-c]pyrido[4′,3′:3,4]pyrazolo[1,5-a]azepine-11(12H)-carboxylate (Intermediate 7) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₁H₁₉FN₆O₂, 406.2; m/z found, 407.1 [M+H]⁺. ¹H NMR (400 MHz, CDCl₃) 8.20-8.19 (m, 1H), 7.84-7.79 (m, 1H), 7.67-7.59 (m, 1H), 7.17 (d, J=8.7 Hz, 1H), 6.62-6.58 (m, 1H), 5.17-5.11 (m, 1H), 4.94 (s, 1H), 4.60 (d, J=15.0 Hz, 1H), 4.51 (s, 2H), 3.09-3.00 (m, 1H), 2.91-2.86 (m, 2H), 2.72-2.65 (m, 1H), 2.32-2.24 (m, 2H), 1.22-1.19 (m, 3H).

### Example 15: (10R)—N-(4-Fluoro-3-(trifluoromethyl)phenyl)-10-methyl-5,6,9,10-tetrahydro-4H-isoxazolo[5,4-c]pyrido[4′,3′:3,4]pyrazolo[1,5-a]azepine-11(12H)-carboxamide

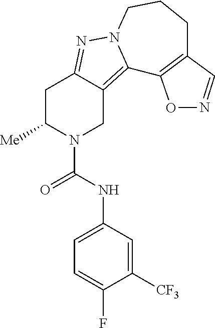 [SMILES: C[C@@H]1Cc2nn3c(c2CN1C(=O)Nc1ccc(F)c(C(F)(F)F)c1)-c1oncc1CCC3]

The title compound was prepared in a manner analogous to Example 1, using (10R)-tert-butyl 10-methyl-5,6,9,10-tetrahydro-4H-isoxazolo[5,4-c]pyrido[4′,3′:3,4]pyrazolo[1,5-a]azepine-11(12H)-carboxylate (Intermediate 7) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (4-fluoro-3-(trifluoromethyl)phenyl)carbamate instead of phenyl (3-cyano-4-fluorophenyl)carbamate in Step B. MS (ESI): mass calcd. for C₂₁H₁₉F₄N₅O₂, 449.2; m/z found, 450.1 [M+H]⁺. ¹H NMR (400 MHz, CDCl₃) 8.20 (s, 1H), 7.75-7.70 (m, 1H), 7.68-7.60 (m, 1H), 7.20-7.12 (m, 1H), 6.63-6.57 (m, 1H), 5.22-5.11 (m, 1H), 4.96 (s, 1H), 4.62 (d, J=15.2 Hz, 1H), 4.52 (t, J=5.0 Hz, 2H), 3.11-3.01 (m, 1H), 2.90 (s, 2H), 2.74-2.65 (m, 1H), 2.35-2.23 (m, 2H), 1.21 (d, J=6.9 Hz, 3H).

### Example 16: (11R)—N-(3-Cyano-4-fluorophenyl)-11-methyl-6,7,10,11-tetrahydro-5H-pyrido[2,3-c]pyrido[4′,3′:3,4]pyrazolo[1,5-a]azepine-12(13H)-carboxamide

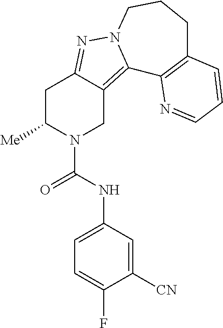 [SMILES: C[C@@H]1Cc2nn3c(c2CN1C(=O)Nc1ccc(F)c(C#N)c1)-c1ncccc1CCC3]

The title compound was prepared in a manner analogous to Example 1, using (11R)-tert-butyl 11-methyl-6,7,10,11-tetrahydro-5H-pyrido[2,3-c]pyrido[4′,3′:3,4]pyrazolo[1,5-a]azepine-12(13H)-carboxylate (Intermediate 8) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₃H₂₁FN₆O, 416.2; m/z found, 417.2 [M+H]⁺. ¹H NMR (400 MHz, CDCl₃) δ=8.60 (dd, J=1.6, 4.8 Hz, 1H), 7.73 (dd, J=2.8, 5.4 Hz, 1H), 7.70-7.63 (m, 2H), 7.25 (dd, J=4.8, 7.6 Hz, 1H), 7.13 (t, J=8.7 Hz, 1H), 6.83 (s, 1H), 5.19-5.05 (m, 1H), 4.97 (d, J=15.3 Hz, 1H), 4.54 (d, J=15.3 Hz, 1H), 4.32-4.22 (m, 2H), 3.10 (dd, J=5.9, 15.8 Hz, 1H), 2.81 (t, J=6.9 Hz, 2H), 2.73 (d, J=16.3 Hz, 1H), 2.49-2.38 (m, 2H), 1.27 (d, J=6.8 Hz, 3H).

### Example 17: (11R)—N-(4-Fluoro-3-(trifluoromethyl)phenyl)-11-methyl-6,7,10,11-tetrahydro-5H-pyrido[2,3-c]pyrido[4′,3′:3,4]pyrazolo[1,5-a]azepine-12(13H)-carboxamide

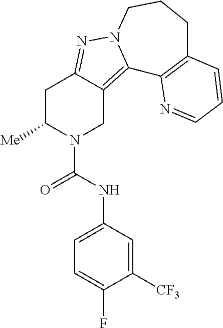 [SMILES: C[C@@H]1Cc2nn3c(c2CN1C(=O)Nc1ccc(F)c(C(F)(F)F)c1)-c1ncccc1CCC3]

The title compound was prepared in a manner analogous to Example 1, using (11R)-tert-butyl 11-methyl-6,7,10,11-tetrahydro-5H-pyrido[2,3-c]pyrido[4′,3′:3,4]pyrazolo[1,5-a]azepine-12(13H)-carboxylate (Intermediate 8) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (4-fluoro-3-(trifluoromethyl)phenyl)carbamate instead of phenyl (3-cyano-4-fluorophenyl)carbamate in Step B. MS (ESI): mass calcd. for C₂₃H₂₁F₄N₅O, 459.2; m/z found, 460.1 [M+H]⁺. ¹H NMR (400 MHz, CDCl₃) δ=8.60 (dd, J=1.7, 4.8 Hz, 1H), 7.68-7.59 (m, 3H), 7.24 (dd, J=4.8, 7.6 Hz, 1H), 7.12 (t, J=9.4 Hz, 1H), 6.73 (s, 1H), 5.15-5.04 (m, 1H), 4.96 (d, J=15.3 Hz, 1H), 4.56 (d, J=15.4 Hz, 1H), 4.28 (t, J=6.8 Hz, 2H), 3.11 (dd, J=6.1, 15.5 Hz, 1H), 2.81 (t, J=6.9 Hz, 2H), 2.73 (d, J=15.7 Hz, 1H), 2.47-2.40 (m, 2H), 1.27 (d, J=7.0 Hz, 3H).

### Example 18: (10R)—N-(3-Cyano-4-fluorophenyl)-10-methyl-5,6,9,10-tetrahydro-4H-isoxazolo[3,4-c]pyrido[4′,3′:3,4]pyrazolo[1,5-a]azepine-11(12H)-carboxamide

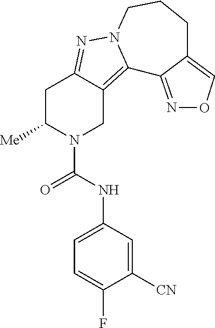 [SMILES: C[C@@H]1Cc2nn3c(c2CN1C(=O)Nc1ccc(F)c(C#N)c1)-c1nocc1CCC3]

The title compound was prepared in a manner analogous to Example 1, using (10R)-tert-butyl 10-methyl-5,6,9,10-tetrahydro-4H-isoxazolo[3,4-c]pyrido[4′,3′:3,4]pyrazolo[1,5-a]azepine-11(12H)-carboxylate (Intermediate 9) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₁H₁₉FN₆O₂, 406.2; m/z found, 407.1 [M+H]⁺. ¹H NMR (400 MHz, CDCl₃) 8.32 (s, 1H), 7.81-7.77 (m, 1H), 7.68-7.61 (m, 1H), 7.18-7.10 (m, 1H), 6.76-6.65 (m, 1H), 5.26-5.12 (m, 1H), 4.92-4.78 (m, 1H), 4.64-4.46 (m, 3H), 3.13-2.92 (m, 3H), 2.77-2.63 (m, 1H), 2.32-2.18 (m, 2H), 1.22-1.17 (m, 3H).

### Example 19: (10R)—N-(4-Fluoro-3-(trifluoromethyl)phenyl)-10-methyl-5,6,9,10-tetrahydro-4H-isoxazolo[3,4-c]pyrido[4′,3′:3,4]pyrazolo[1,5-a]azepine-11(12H)-carboxamide

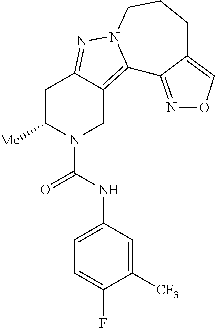 [SMILES: C[C@@H]1Cc2nn3c(c2CN1C(=O)Nc1ccc(F)c(C(F)(F)F)c1)-c1nocc1CCC3]

The title compound was prepared in a manner analogous to Example 1, using (10R)-tert-butyl 10-methyl-5,6,9,10-tetrahydro-4H-isoxazolo[3,4-c]pyrido[4′,3′:3,4]pyrazolo[1,5-a]azepine-11(12H)-carboxylate (Intermediate 9) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (4-fluoro-3-(trifluoromethyl)phenyl)carbamate instead of phenyl (3-cyano-4-fluorophenyl)carbamate in Step B. MS (ESI): mass calcd. for C₂₁H₁₉F₄N₅O₂, 449.2; m/z found, 450.1 [M+H]⁺; ¹H NMR (400 MHz, CDCl₃) 8.33 (s, 1H), 7.74-7.69 (m, 1H), 7.66-7.60 (m, 1H), 7.14 (t, J=9.5 Hz, 1H), 6.66 (s, 1H), 5.20 (t, J=7.0 Hz, 1H), 4.87 (d, J=15.3 Hz, 1H), 4.61-4.50 (m, 3H), 3.11-2.96 (m, 3H), 2.70 (d, J=15.6 Hz, 1H), 2.32-2.22 (m, 2H), 1.20 (d, J=6.9 Hz, 3H).

### Example 20: N-(3-Chloro-4-fluorophenyl)-6,7,10,11-tetrahydro-5H-pyrido[4′,3′:3,4]-pyrazolo[1,5-a][1,2,4]triazolo[3,4-c][1,4]diazepine-12(13H)-carboxamide

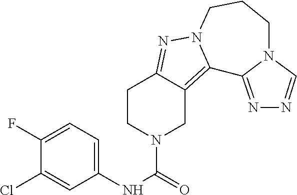 [SMILES: O=C(Nc1ccc(F)c(Cl)c1)N1CCc2nn3c(c2C1)-c1nncn1CCC3]

The title compound was prepared in a manner analogous to Example 1, using tert-butyl 6,7,10,11-tetrahydro-5H-pyrido[4′,3′:3,4]pyrazolo[1,5-a][1,2,4]triazolo[3,4-c][1,4]diazepine-12(13H)-carboxylate (Intermediate 10) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₁₈H₁₇ClFN₇O, 401.1; m/z found, 402 [M+H]⁺. ¹H NMR (400 MHz, CDCl₃) δ=8.19 (s, 1H), 7.65 (dd, J=2.6, 6.7 Hz, 1H), 7.29 (dd, J=2.8, 4.1 Hz, 1H), 7.00-7.10 (m, 2H), 4.86 (s, 2H), 4.63-4.70 (m, 2H), 4.39-4.45 (m, 2H), 3.91 (t, J=5.8 Hz, 2H), 2.87 (t, J=5.8 Hz, 2H), 2.49-2.59 (m, 2H).

### Example 21: N-(3-Chloro-4-fluorophenyl)-3-methyl-6,7,10,11-tetrahydro-5H-pyrido-[4′,3′:3,4]pyrazolo[1,5-a][1,2,4]triazolo[3,4-c][1,4]diazepine-12(13H)-carboxamide

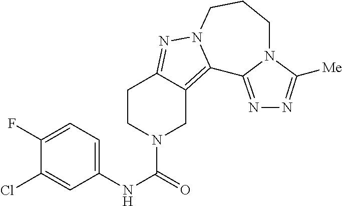 [SMILES: Cc1nnc2n1CCCn1nc3c(c1-2)CN(C(=O)Nc1ccc(F)c(Cl)c1)CC3]

The title compound was prepared in a manner analogous to Example 1, using tert-butyl 3-methyl-6,7,10,11-tetrahydro-5H-pyrido[4′,3′:3,4]pyrazolo[1,5-a][1,2,4]triazolo[3,4-c][1,4]diazepine-12(13H)-carboxylate (Intermediate 11) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₁₉H₁₉ClFN₇O, 415.1; m/z found, 416 [M+H]⁺. ¹H NMR (400 MHz, CDCl₃) δ=7.66 (dd, J=2.7, 6.6 Hz, 1H), 7.27-7.31 (m, 1H), 7.12 (s, 1H), 7.05 (t, J=8.8 Hz, 1H), 4.83 (s, 2H), 4.61-4.66 (m, 2H), 4.18-4.23 (m, 2H), 3.91 (t, J=5.8 Hz, 2H), 2.85 (t, J=5.7 Hz, 2H), 2.49-2.56 (m, 5H).

### Example 22: (R)—N-(3-Chloro-4-fluorophenyl)-11-methyl-6,7,10,11-tetrahydro-5H-pyrido[4′,3′:3,4]pyrazolo[1,5-a][1,2,4]triazolo[3,4-c][1,4]diazepine-12(13H)-carboxamide

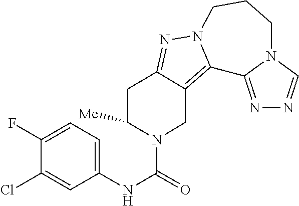 [SMILES: C[C@@H]1Cc2nn3c(c2CN1C(=O)Nc1ccc(F)c(Cl)c1)-c1nncn1CCC3]

The title compound was prepared in a manner analogous to Example 1, using (11R)-tert-butyl 11-methyl-6,7,10,11-tetrahydro-5H-pyrido[4′,3′:3,4]pyrazolo[1,5-a][1,2,4]triazolo[3,4-c][1,4]diazepine-12(13H)-carboxylate (Intermediate 12) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₁₉H₁₉ClFN₇O, 415.1; m/z found, 416 [M+H]⁺. ¹H NMR (400 MHz, CDCl₃) δ=8.20 (s, 1H), 7.67 (dd, J=2.6, 6.5 Hz, 1H), 7.28-7.31 (m, 1H), 7.06 (t, J=8.8 Hz, 1H), 6.96 (br s, 1H), 5.25 (quin, J=6.5 Hz, 1H), 5.00 (d, J=15.8 Hz, 1H), 4.58-4.74 (m, 3H), 4.40-4.49 (m, 2H), 3.06 (dd, J=5.9, 15.9 Hz, 1H), 2.69 (d, J=15.8 Hz, 1H), 2.55 (br d, J=3.3 Hz, 2H), 1.18 (d, J=7.0 Hz, 3H).

### Example 23: (11R)—N-(3-Chloro-4-fluorophenyl)-11-methyl-6,7,10,11-tetrahydro-5H-pyrido[4′,3′:3,4]pyrazolo[1,5-a][1,2,4]triazolo[3,4-c][1,4]diazepine-12(13H)-carboxamide

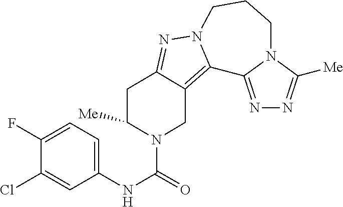 [SMILES: Cc1nnc2n1CCCn1nc3c(c1-2)CN(C(=O)Nc1ccc(F)c(Cl)c1)[C@H](C)C3]

The title compound was prepared in a manner analogous to Example 1, using (11R)-tert-butyl 3,11-dimethyl-6,7,10,11-tetrahydro-5H-pyrido[4′,3′:3,4]pyrazolo[1,5-a][1,2,4]triazolo[3,4-c][1,4]diazepine-12(13H)-carboxylate (Intermediate 13) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₂₀H₂₁ClFN₇O, 429.1; m/z found, 430 [M+H]⁺. ¹H NMR (400 MHz, CDCl₃) δ=7.68 (dd, J=2.7, 6.6 Hz, 1H), 7.28-7.32 (m, 1H), 7.06 (t, J=8.8 Hz, 1H), 6.98 (s, 1H), 4.97 (m, 1H), 4.57-4.68 (m, 3H), 4.20-4.25 (m, 2H), 3.05 (m, 1H), 2.68 (m, 1H), 2.54 (s, 5H), 1.17 (d, J=6.9 Hz, 3H).

### Example 24: N-(3-Cyano-4-fluorophenyl)-6,7,10,11-tetrahydro-5H-pyridazino[3,4-c]pyrido[4′,3′:3,4]pyrazolo[1,5-a]azepine-12(13H)-carboxamide

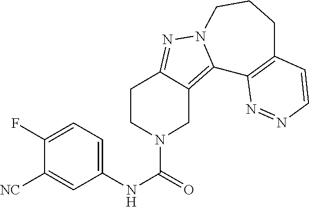 [SMILES: N#Cc1cc(NC(=O)N2CCc3nn4c(c3C2)-c2nnccc2CCC4)ccc1F]

The title compound was prepared in a manner analogous to Example 1, using tert-butyl 10-methyl-11-oxo-8-(1H-pyrazol-3-yl)-3,4,8,9,10,11-hexahydro-1H-pyrido[4′,3′:3,4]pyrazolo[1,5-a][1,4]diazepine-2(7H)-carboxylate (Intermediate 15) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A. MS (ESI): mass calcd. for C₂₁H₁₈FN₇O, 403.1; m/z found, 404 [M+H]⁺. ¹H NMR (400 MHz, CDCl₃) δ 9.04 (d, J=5.1 Hz, 1H), 7.75-7.82 (m, 1H), 7.64 (ddd, J=2.8, 4.6, 9.2 Hz, 1H), 7.43 (d, J=5.1 Hz, 1H), 7.12 (t, J=8.7 Hz, 1H), 6.90 (s, 1H), 4.89 (s, 2H), 4.40 (t, J=6.5 Hz, 2H), 3.94 (t, J=5.8 Hz, 2H), 2.87-3.00 (m, 4H), 2.44 (t, J=6.5 Hz, 2H) Example 25: N-(3-Chloro-4-fluorophenyl)-4,5,6,9,10,12-hexahydropyrazolo[3,4-c]pyrido[4′,3′:3,4]pyrazolo[1,5-a]azepine-11(2H)-carboxamide.

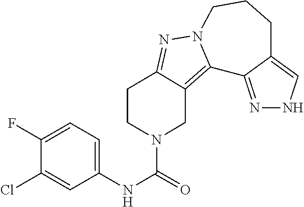 [SMILES: O=C(Nc1ccc(F)c(Cl)c1)N1CCc2nn3c(c2C1)-c1n[nH]cc1CCC3]

The title compound was prepared in a manner analogous to Example 1, using tert-butyl 4,5,6,9,10,12-hexahydropyrazolo[3,4-c]pyrido[4′,3′:3,4]pyrazolo[1,5-a]azepine-11(2H)-carboxylate (Intermediate 16) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A and using phenyl (3-chloro-4-fluorophenyl)carbamate instead of phenyl N-(3-cyano-4-fluoro-phenyl)carbamate in Step B. MS(ESI): mass calcd. for C₁₉H₁₈ClFN₆O, 400.1; m/z found, 401 [M+H]⁺. ¹H NMR (400 MHz, CDCl₃) δ 7.54 (dd, J=2.6, 6.4 Hz, 1H), 7.45 (s, 1H), 7.21-7.26 (m, 1H), 7.00-7.08 (m, 1H), 6.63-6.70 (m, 1H), 4.76 (s, 2H), 4.44-4.57 (m, 2H), 3.87 (t, J=5.8 Hz, 2H), 2.92-3.03 (m, 2H), 2.86 (t, J=5.8 Hz, 2H), 2.16-2.30 (m, 2H).

### Example 26: N-(3-Cyano-4-fluorophenyl)-4,5,6,9,10,12-hexahydropyrazolo[3,4-c]pyrido[4′,3′:3,4]pyrazolo[1,5-a]azepine-11(2H)-carboxamide

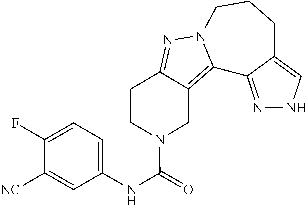 [SMILES: N#Cc1cc(NC(=O)N2CCc3nn4c(c3C2)-c2n[nH]cc2CCC4)ccc1F]

The title compound was prepared in a manner analogous to Example 1, using tert-butyl 4,5,6,9,10,12-hexahydropyrazolo[3,4-c]pyrido[4′,3′:3,4]pyrazolo[1,5-a]azepine-11(2H)-carboxylate (Intermediate 16) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A. MS(ESI): mass calcd. for C₂₀H₁₈FN₇O, 391.1; m/z found, 392 [M+H]⁺. ¹H NMR (400 MHz, CD₃OD) δ 7.79-7.85 (m, 1H), 7.71 (ddd, J=2.8, 4.7, 9.2 Hz, 1H), 7.56 (s, 1H), 7.27 (t, J=9.0 Hz, 1H), 4.80 (s, 2H), 4.39-4.46 (m, 2H), 3.80-3.89 (m, 2H), 2.93-3.02 (m, 2H), 2.80 (t, J=5.7 Hz, 2H), 2.12-2.23 (m, 2H).

### Example 27: N-(3-Cyano-4-fluorophenyl)-6,7,10,11-tetrahydro-5H-pyrido[2,3-c]pyrido[4′,3′:3,4]pyrazolo[1,5-a]azepine-12(13H)-carboxamide

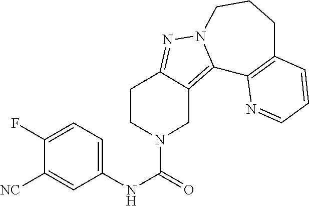 [SMILES: N#Cc1cc(NC(=O)N2CCc3nn4c(c3C2)-c2ncccc2CCC4)ccc1F]

The title compound was prepared in a manner analogous to Example 1, using tert-butyl 11-oxo-3,4,8,9,10,11-hexahydro-1H-pyrido[4′,3′:3,4]pyrazolo[1,5-a]azepine-2(7H)-carboxylate (Intermediate 17) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₂H₁₉FN₆O, 402.16; m/z found, 403.2 [M+H]⁺. ¹H NMR (400 MHz, CDCl₃) δ=8.59 (dd, J=1.6, 4.8 Hz, 1H), 7.72 (dd, J=2.8, 5.4 Hz, 1H), 7.69-7.63 (m, 2H), 7.27-7.23 (m, 1H), 7.13 (t, J=8.8 Hz, 1H), 6.87 (s, 1H), 4.79 (s, 2H), 4.26 (t, J=6.8 Hz, 2H), 3.91 (t, J=5.8 Hz, 2H), 2.92 (t, J=5.8 Hz, 2H), 2.81 (t, J=6.8 Hz, 2H), 2.46-2.39 (m, 2H).

### Example 28: N-(4-Fluoro-3-(trifluoromethyl)phenyl)-6,7,10,11-tetrahydro-5H-pyrido[2,3-c]pyrido[4′,3′:3,4]pyrazolo[1,5-a]azepine-12(13H)-carboxamide

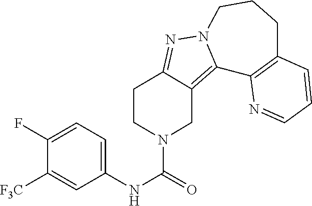 [SMILES: O=C(Nc1ccc(F)c(C(F)(F)F)c1)N1CCc2nn3c(c2C1)-c1ncccc1CCC3]

The title compound was prepared in a manner analogous to Example 1, using tert-butyl 11-oxo-3,4,8,9,10,11-hexahydro-1H-pyrido[4′,3′:3,4]pyrazolo[1,5-a]azepine-2(7H)-carboxylate (Intermediate 17) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (4-fluoro-3-(trifluoromethyl)phenyl)carbamate instead of phenyl (3-cyano-4-fluorophenyl)carbamate in Step B. MS (ESI): mass calcd. for C₂₂H₁₉F₄N₅O, 445.2; m/z found, 446.1 [M+H]⁺. ¹H NMR (400 MHz, CDCl₃) δ=8.59 (dd, J=1.6, 4.8 Hz, 1H), 7.67-7.59 (m, 3H), 7.24 (dd, J=4.8, 7.6 Hz, 1H), 7.12 (t, J=9.4 Hz, 1H), 6.74 (s, 1H), 4.79 (s, 2H), 4.26 (t, J=6.8 Hz, 2H), 3.91 (t, J=6.0 Hz, 2H), 2.92 (t, J=6.0 Hz, 2H), 2.81 (t, J=6.8 Hz, 2H), 2.46-2.39 (m, 2H).

### Example 29: N-(3-Chloro-4-fluorophenyl)-2-methyl-4,5,6,9,10,12-hexahydropyrazolo[3,4-c]pyrido[4′,3′:3,4]pyrazolo[1,5-a]azepine-11(2H)-carboxamide

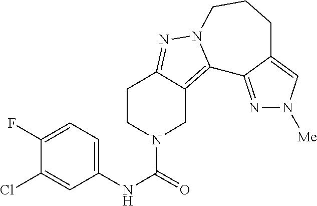 [SMILES: Cn1cc2c(n1)-c1c3c(nn1CCC2)CCN(C(=O)Nc1ccc(F)c(Cl)c1)C3]

The title compound was prepared in a manner analogous to Example 1, using tert-butyl 2-methyl-4,5,6,9,10,12-hexahydropyrazolo[3,4-c]pyrido[4′,3′:3,4]pyrazolo[1,5-a]azepine-11(2H)-carboxylate (Intermediate 18) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A and using phenyl (3-chloro-4-fluorophenyl)carbamate instead of phenyl N-(3-cyano-4-fluoro-phenyl)carbamate in Step B. MS(ESI): mass calcd. for C₂OH₂OClFN₆O, 414.1; m/z found, 415.1 [M+H]⁺. ¹H NMR (400 MHz, CDCl₃) δ 7.57 (dd, J=2.6, 6.5 Hz, 1H), 7.21-7.26 (m, 2H), 7.06 (t, J=8.8 Hz, 1H), 6.62 (s, 1H), 4.74 (s, 2H), 4.39-4.55 (m, 2H), 3.93 (s, 3H), 3.86 (s, 2H), 2.85 (s, 4H), 2.18 (br s, 2H).

### Example 30: N-(3-Chloro-4-fluorophenyl)-1-methyl-4,5,6,9,10,12-hexahydropyrazolo[3,4-c]pyrido[4′3′:3,4]pyrazolo[1,5-a]azepine-11(1H)-carboxamide

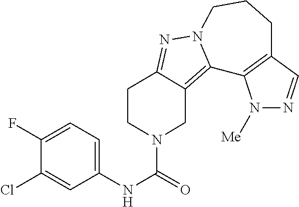 [SMILES: Cn1ncc2c1-c1c3c(nn1CCC2)CCN(C(=O)Nc1ccc(F)c(Cl)c1)C3]

The title compound was prepared in a manner analogous to Example 1, using tert-butyl 1-methyl-4,5,6,9,10,12-hexahydropyrazolo[3,4-c]pyrido[4′,3′:3,4]pyrazolo[1,5-a]azepine-11(1H)-carboxylate (Intermediate 19) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₂₀H₂₀ClFN₆O, 414.1; m/z found, 415.1 [M+H]⁻. ¹H NMR (400 MHz, CDCl₃) δ 7.49-7.56 (m, 1H), 7.44 (s, 1H), 7.14-7.22 (m, 1H), 7.02-7.11 (m, 1H), 6.41 (s, 1H), 4.64 (s, 2H), 4.16-4.25 (m, 2H), 3.94 (s, 3H), 3.84 (s, 2H), 2.91-3.00 (m, 2H), 2.73 (s, 2H), 2.16-2.29 (m, 2H).

### Example 31: N-(3-Chloro-4-fluorophenyl)-5,6,9,10-tetrahydro-4H-isoxazolo[3,4-c]pyrido[4′,3′:3,4]pyrazolo[1,5-a]azepine-11(12H)-carboxamide

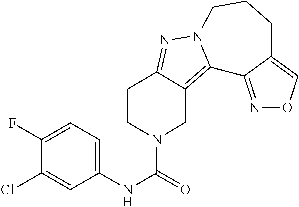 [SMILES: O=C(Nc1ccc(F)c(Cl)c1)N1CCc2nn3c(c2C1)-c1nocc1CCC3]

The title compound was prepared in a manner analogous to Example 1, using tert-butyl 5,6,9,10-tetrahydro-4H-isoxazolo[3,4-c]pyrido[4′,3′:3,4]pyrazolo[1,5-a]azepine-11(12H)-carboxylate (Intermediate 20) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₁₉H₁₇ClFN₅O₂, 401.1; m/z found, 402.1 [M+H]⁺. ¹H NMR (400 MHz, CDCl₃) δ 8.32 (s, 1H), 7.60 (dd, J=2.2, 6.5 Hz, 1H), 7.24 (br d, J=3.3 Hz, 1H), 7.06 (t, J=8.7 Hz, 1H), 6.62 (s, 1H), 4.73 (s, 2H), 4.53-4.61 (m, 2H), 3.90 (t, J=5.7 Hz, 2H), 2.93-3.03 (m, 2H), 2.87 (t, J=5.7 Hz, 2H), 2.19-2.31 (m, 2H).

### Example 32: N-(3-Chloro-4-fluorophenyl)-5,6,9,10-tetrahydro-4H-isoxazolo[5,4-c]pyrido[4′,3′:3,4]pyrazolo[1,5-a]azepine-11(12H)-carboxamide

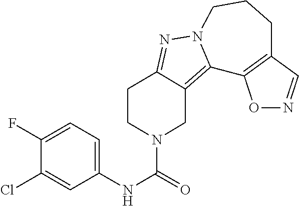 [SMILES: O=C(Nc1ccc(F)c(Cl)c1)N1CCc2nn3c(c2C1)-c1oncc1CCC3]

The title compound was prepared in a manner analogous to Example 1, using tert-butyl 5,6,9,10-tetrahydro-4H-isoxazolo[5,4-c]pyrido[4′,3′:3,4]pyrazolo[1,5-a]azepine-11(12H)-carboxylate (Intermediate 21) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₁₉H₁₇ClFN₅O₂, 401.1; m/z found, 402.1 [M+H]⁺. ¹H NMR (400 MHz, CDCl₃) δ 8.19 (s, 1H), 7.60 (dd, J=2.7, 6.5 Hz, 1H), 7.21-7.26 (m, 1H), 7.08 (t, J=8.7 Hz, 1H), 6.54 (s, 1H), 4.83 (s, 2H), 4.46-4.53 (m, 2H), 3.88 (t, J=5.8 Hz, 2H), 2.87 (td, J=6.0, 8.2 Hz, 4H), 2.27 (br dd, J=3.8, 6.1 Hz, 2H).

### Example 33: N-(3-Cyano-4-fluorophenyl)-5,6,9,10-tetrahydro-4H-isoxazolo[5″,4″:3′,4′]-cyclohepta[1′,2′:3,4]pyrazolo[1,5-a]pyrazine-11(12H)-carboxamide

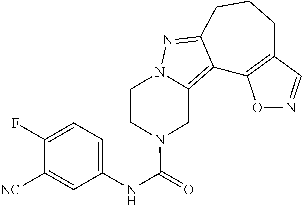 [SMILES: N#Cc1cc(NC(=O)N2CCn3nc4c(c3C2)-c2oncc2CCC4)ccc1F]

Step A. 5,6,9,10,11,12-Hexahydro-4H-isoxazolo[5″,4″:3′,4′]cyclohepta[1′,2′:3,4]pyrazolo[1,5-a]pyrazine. To a solution of tert-butyl 5,6,9,10-tetrahydro-4H-isoxazolo [5″,4″:3′,4′]cyclohepta[1′,2′:3,4]pyrazolo[1,5-a]pyrazine-11(12H)-carboxylate (0.07 g, 211.88 umol) in DCM (2 mL) was added TFA (1.54 g, 13.51 mmol, 1 mL) and the mixture was stirred at 20° C. for 1 h. The mixture was concentrated under reduced pressure to give title compound (0.073 g, crude, TFA salt) as a yellow oil, which was used directly for the next step.

Step B. N-(3-Cyano-4-fluorophenyl)-5,6,9,10-tetrahydro-4H-isoxazolo[5″,4″:3′,4′]cyclohepta[1′,2′:3,4]pyrazolo[1,5-a]pyrazine-11(12H)-carboxamide. A mixture of 5,6,9,10,11,12-hexahydro-4H isoxazolo[5″,4″:3′,4′]cyclohepta [1′,2′:3,4]pyrazolo [1,5-a]pyrazine (0.073 g, TFA salt), phenyl N-(3-cyano-4-fluoro-phenyl)carbamate (54.33 mg, 212.03 umol) and Et₃N (107.28 mg, 1.06 mmol, 147.56 uL) in DCM (4 mL) was stirred at 20° C. for 16 h. The mixture was concentrated under reduced pressure. The residue was purified by prep-HPLC (condition A) to give title compound (0.048 g, 120.86 umol, 57.00% yield, 98.8% purity) as a white solid. MS (ESI): mass calcd. for C₂₀H₁₇FN₆O₂ 392.1; m/z found, 393.1 [M+H]⁺. ¹H NMR (400 MHz, DMSO-d₆) δ 9.32 (s, 1H), 8.41 (s, 1H), 7.95-7.93 (m, 1H), 7.78-7.78 (m, 1H), 7.48-7.44 (m, 1H), 5.01 (s, 2H), 4.17-4.14 (m, 2H), 3.99-3.97 (m, 2H), 2.93-2.90 (m, 2H), 2.76-2.73 (m, 2H), 1.91-1.89 (m, 2H).

### Example 34: N-(3-Cyano-4-fluorophenyl)-5,6,9,10-tetrahydro-4H-isoxazolo[5″,4″:3′,4′]-cyclohepta[1′,2′:3,4]pyrazolo[1,5-a]pyrazine-11(12H)-carboxamide

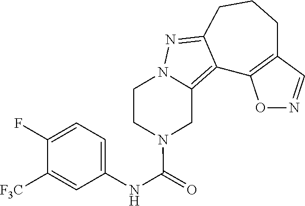 [SMILES: O=C(Nc1ccc(F)c(C(F)(F)F)c1)N1CCn2nc3c(c2C1)-c1oncc1CCC3]

The title compound was prepared in a manner analogous to Example 1, step 2, using phenyl (4-fluoro-3-(trifluoromethyl)phenyl)carbamate instead of phenyl N-(3-cyano-4-fluoro-phenyl)carbamate. MS (ESI): mass calcd. for C₂₀H₁₇F₄N₅O₂ 435.1; m/z found, 436.1 [M+H]⁺. ¹H NMR (400 MHz, DMSO-d₆) δ 9.30 (s, 1H), 8.41 (s, 1H), 7.93-7.90 (m, 1H), 7.80-7.77 (m, 1H), 7.45-7.41 (m, 1H), 5.01 (s, 2H), 4.17-4.00 (m, 2H), 3.99-3.98 (m, 2H), 2.93-2.90 (m, 2H), 2.75-2.73 (m, 2H), 1.91-1.89 (m, 2H).

### Example 35: N-(3-Cyano-4-fluorophenyl)-5,6,9,10-tetrahydro-4H-isoxazolo[3″,4″:3′,4′]-cyclohepta[1′,2′:3,4]pyrazolo[1,5-a]pyrazine-11(12H)-carboxamide

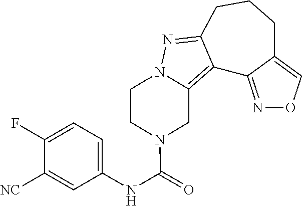 [SMILES: N#Cc1cc(NC(=O)N2CCn3nc4c(c3C2)-c2nocc2CCC4)ccc1F]

The title compound was prepared in a manner analogous to Example 1, using tert-butyl 5,6,9,10-tetrahydro-4H-isoxazolo[3″,4″:3′,4′]cyclohepta[1′,2′:3,4]pyrazolo[1,5-a]pyrazine-11(12H)-carboxylate instead of tert-butyl 5,6,9,10-tetrahydro-4H-isoxazolo[5″,4″:3′,4′]cyclohepta[1′,2′:3,4]pyrazolo[1,5-a]pyrazine-11(12H)-carboxylate. MS (ESI): mass calcd. for C₂₀H₁₇FN₆O₂ 392.1; m/z found, 393.1 [M+H]⁺. ¹H NMR (400 MHz, DMSO-d₆) δ 9.31 (s, 1H), 8.65 (s, 1H), 7.94-7.92 (m, 1H), 7.78-7.77 (m, 1H), 7.48-7.43 (m, 1H), 4.90 (s, 2H), 4.18-4.15 (m, 2H), 3.99-3.95 (m, 2H), 2.97-2.95 (m, 2H), 2.82-2.79 (m, 2H), 1.88-1.86 (m, 2H).

### Example 36: N-(4-Fluoro-3-(trifluoromethyl)phenyl)-5,6,9,10-tetrahydro-4H-isoxazolo[3″,4″:3′,4′]cyclohepta[1′,2′:3,4]pyrazolo[1,5-a]pyrazine-11(12H)-carboxamide

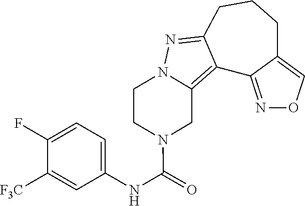 [SMILES: O=C(Nc1ccc(F)c(C(F)(F)F)c1)N1CCn2nc3c(c2C1)-c1nocc1CCC3]

The title compound was prepared in a manner analogous to Example 1, step 2, except using 5,6,9,10,11,12-hexahydro-4H-isoxazolo[3″,4″:3′,4′]cyclohepta[1′,2′:3,4]pyrazolo[1,5-a]pyrazine to react with phenyl (4-fluoro-3-(trifluoromethyl)phenyl)carbamate instead of 5,6,9,10,11,12-hexahydro-4H-isoxazolo[5″,4″:3′,4′]cyclohepta[1′,2′:3,4]pyrazolo[1,5-a]pyrazine to react with phenyl N-(3-cyano-4-fluoro-phenyl)carbamate. MS (ESI): mass calcd. for C₂₀H₁₇F₄N₅O₂ 435.1; m/z found, 436.1 [M+H]⁺. ¹H NMR (400 MHz, DMSO-d₆) δ 9.29 (s, 1H), 8.65 (s, 1H), 7.92-7.90 (m, 1H), 7.79-7.77 (m, 1H), 7.45-7.40 (m, 1H), 4.90 (s, 2H), 4.18-4.16 (m, 2H), 4.00-3.99 (m, 2H), 2.98-2.95 (m, 2H), 2.81-2.80 (m, 2H), 1.89-1.86 (m, 2H).

### Biological Data

### HBV Replication Inhibition Assay

HBV replication inhibition by the disclosed compounds were determined in cells infected or transfected with HBV or cells with stably integrated HBV, such as HepG2.2.15 cells (Sells et al. 1987). In this example, HepG2.2.15 cells were maintained in cell culture medium containing 10% fetal bovine serum (FBS), Geneticin, L-glutamine, penicillin and streptomycin. HepG2.2.15 cells were seeded in 96-well plates at a density of 40,000 cells/well and were treated with serially diluted compounds at a final DMSO concentration of 0.5% either alone or in combination by adding drugs in a checker box format. Cells were incubated with compounds for three days, after which medium was removed and fresh medium containing compounds was added to cells and incubated for another three days. At day 6, supernatant was removed and treated with DNase at 37° C. for 60 minutes, followed by enzyme inactivation at 75° C. for 15 minutes. Encapsidated HBV DNA was released from the virions and covalently linked HBV polymerase by incubating in lysis buffer (Affymetrix QS0010) containing 2.5 μg proteinase K at 50° C. for 40 minutes. HBV DNA was denatured by addition of 0.2 M NaOH and detected using a branched DNA (BDNA) QuantiGene assay kit according to manufacturer recommendation (Affymetrix). HBV DNA levels were also quantified using qPCR, based on amplification of encapsidated HBV DNA extraction with QuickExtraction Solution (Epicentre Biotechnologies) and amplification of HBV DNA using HBV specific PCR probes that can hybridize to HBV DNA and a fluorescently labeled probe for quantitation. In addition, cell viability of HepG2.2.15 cells incubated with test compounds alone or in combination was determined by using CellTitre-Glo reagent according to the manufacturer protocol (Promega). The mean background signal from wells containing only culture medium was subtracted from all other samples, and percent inhibition at each compound concentration was calculated by normalizing to signals from HepG2.2.15 cells treated with 0.5% DMSO using equation E1.

$$\% inhibition = ( DMSOave - Xi ) / DMSOave \times 100 \% \qquad E 1$$

where DMSOave is the mean signal calculated from the wells that were treated with DMSO control (0% inhibition control) and Xi is the signal measured from the individual wells. EC₅₀ values, effective concentrations that achieved 50% inhibitory effect, were determined by non-linear fitting using Graphpad Prism software (San Diego, Calif.) and equation E2.

$$Y = Y min + ( Y max - Y min ) / ( 1 + 10 ( Log EC 50 - X ) \times HillSlope ) \qquad E 2$$

where Y represents percent inhibition values and X represents the logarithm of compound concentrations.

Selected disclosed compounds were assayed in the HBV replication assay (BDNA assay), as described above, and a representative group of these active compounds is shown in Table 3. Table 3 shows EC₅₀ values obtained by the BDNA assay for a group of select compounds.

TABLE 3
Activity in BDNA-assay (EC₅₀)
Biological Example 1

| Ex | | EC₅₀ |
|---|---|---|
| # | Compound name | (nM) |
| 1 | N-(3-Cyano-4-fluorophenyl)-5-methylene-5,6,9,10-tetrahydro-4H- | 64 |
| | isoxazolo[3,4-c]pyrido[4′,3′:3,4]pyrazolo[1,5-a]azepine-11(12H)- | |
| | carboxamide; | |
| 2 | N-(4-Fluoro-3-(trifluoromethyl)phenyl)-5-methylene-5,6,9,10- | 75 |
| | tetrahydro-4H-isoxazolo[3,4-c]pyrido[4′,3′:3,4]pyrazolo[1,5- | |
| | a]azepine-11(12H)-carboxamide; | |
| 3 | N-(3-Cyano-4-fluorophenyl)-5-(hydroxymethyl)-5,6,9,10- | 23 |
| | tetrahydro-4H-isoxazolo[3,4-c]pyrido[4′,3′:3,4]pyrazolo[1,5- | |
| | a]azepine-11(12H)-carboxamide; | |
| 4 | N-(4-Fluoro-3-(trifluoromethyl)phenyl)-5-(hydroxymethyl)- | 57 |
| | 5,6,9,10-tetrahydro-4H-isoxazolo[3,4- | |
| | c]pyrido[4′,3′:3,4]pyrazolo[1,5-a]azepine-11(12H)-carboxamide; | |
| 5 | (5S*)-N-(3-Cyano-4-fluorophenyl)-5-((2,2- | 82 |
| | difluoroethoxy)methyl)-5,6,9,10-tetrahydro-4H-isoxazolo[3,4- | |
| | c]pyrido[4′,3′:3,4]pyrazolo[1,5-a]azepine-11(12H)-carboxamide; | |
| 6 | (5S*)-5-((2,2-Difluoroethoxy)methyl)-N-(4-fluoro-3- | 45 |
| | (trifluoromethyl)phenyl)-5,6,9,10-tetrahydro-4H-isoxazolo[3,4- | |
| | c]pyrido[4′,3′:3,4]pyrazolo[1,5-a]azepine-11(12H)-carboxamide; | |
| 7 | (5R*)-N-(3-Cyano-4-fluorophenyl)-5-((2,2- | 21 |
| | difluoroethoxy)methyl)-5,6,9,10-tetrahydro-4H-isoxazolo[3,4- | |
| | c]pyrido[4′,3′:3,4]pyrazolo[1,5-a]azepine-11(12H)-carboxamide; | |
| 8 | (5R*)-5-((2,2-Difluoroethoxy)methyl)-N-(4-fluoro-3- | 26 |
| | (trifluoromethyl)phenyl)-5,6,9,10-tetrahydro-4H-isoxazolo[3,4- | |
| | c]pyrido[4′,3′:3,4]pyrazolo[1,5-a]azepine-11(12H)-carboxamide; | |
| 9 | N-(3-Cyano-4-fluorophenyl)-5-methylene-5,6,9,10-tetrahydro-4H- | 800 |
| | isoxazolo[5,4-c]pyrido[4′,3′:3,4]pyrazolo[1,5-a]azepine-11(12H)- | |
| | carboxamide; | |
| 10 | N-(4-Fluoro-3-(trifluoromethyl)phenyl)-5-methylene-5,6,9,10- | 350 |
| | tetrahydro-4H-isoxazolo[5,4-c]pyrido[4′,3′:3,4]pyrazolo[1,5- | |
| | a]azepine-11(12H)-carboxamide; | |
| 11 | N-(3-Cyano-4-fluorophenyl)-5-hydroxy-5,6,9,10-tetrahydro-4H- | 43 |
| | isoxazolo[3,4-c]pyrido[4′,3′:3,4]pyrazolo[1,5-a]azepine-11(12H)- | |
| | carboxamide; | |
| 12 | N-(4-Fluoro-3-(trifluoromethyl)phenyl)-5-methyl-5,6,9,10- | 52 |
| | tetrahydro-4H-isoxazolo[3,4-c]pyrido[4′,3′:3,4]pyrazolo[1,5- | |
| | a]azepine-11(12H)-carboxamide; | |
| 13 | N-(3-Cyano-4-fluorophenyl)-5-methyl-5,6,9,10-tetrahydro-4H- | 52 |
| | isoxazolo[3,4-c]pyrido[4′,3′:3,4]pyrazolo[1,5-a]azepine-11(12H)- | |
| | carboxamide; | |
| 14 | (10R)-N-(3-Cyano-4-fluorophenyl)-10-methyl-5,6,9,10-tetrahydro- | 59 |
| | 4H-isoxazolo[5,4-c]pyrido[4′,3′:3,4]pyrazolo[1,5-a]azepine- | |
| | 11(12H)-carboxamide; | |
| 15 | (10R)-N-(4-Fluoro-3-(trifluoromethyl)phenyl)-10-methyl-5,6,9,10- | 83 |
| | tetrahydro-4H-isoxazolo[5,4-c]pyrido[4′,3′:3,4]pyrazolo[1,5- | |
| | a]azepine-11(12H)-carboxamide; | |
| 16 | (11R)-N-(3-Cyano-4-fluorophenyl)-11-methyl-6,7,10,11- | 330 |
| | tetrahydro-5H-pyrido[2,3-c]pyrido[4′,3′:3,4]pyrazolo[1,5- | |
| | a]azepine-12(13H)-carboxamide; | |
| 17 | (11R)-N-(4-Fluoro-3-(trifluoromethyl)phenyl)-11-methyl- | 220 |
| | 6,7,10,11-tetrahydro-5H-pyrido[2,3- | |
| | c]pyrido[4′,3′:3,4]pyrazolo[1,5-a]azepine-12(13H)-carboxamide; | |
| 18 | (10R)-N-(3-Cyano-4-fluorophenyl)-10-methyl-5,6,9,10-tetrahydro- | 15 |
| | 4H-isoxazolo[3,4-c]pyrido[4′,3′:3,4]pyrazolo[1,5-a]azepine- | |
| | 11(12H)-carboxamide; | |
| 19 | (10R)-N-(4- | 27 |
| | Fluoro-3-(trifluoromethyl)phenyl)-10-methyl-5,6,9,10-tetrahydro- | |
| | 4H-isoxazolo[3,4-c]pyrido[4′,3′:3,4]pyrazolo[1,5-a]azepine- | |
| | 11(12H)-carboxamide; | |
| 20 | N-(3-Chloro-4-fluorophenyl)-6,7,10,11-tetrahydro-5H- | 2000 |
| | pyrido[4′,3′:3,4]pyrazolo[1,5-a][1,2,4]triazolo[3,4- | |
| | c][1,4]diazepine-12(13H)-carboxamide; | |
| 21 | N-(3-Chloro-4-fluorophenyl)-3-methyl-6,7,10,11-tetrahydro-5H- | 2700 |
| | pyrido[4′,3′:3,4]pyrazolo[1,5-a][1,2,4]triazolo[3,4- | |
| | c][1,4]diazepine-12(13H)-carboxamide; | |
| 22 | (11R)-N-(3-Chloro-4-fluorophenyl)-11-methyl-6,7,10,11- | >4000 |
| | tetrahydro-5H-pyrido[4′,3′:3,4]pyrazolo[1,5-a][1,2,4]triazolo[3,4-c] | |
| | [1,4]diazepine-12(13H)-carboxamide; | |
| 23 | (11R)-N-(3-Chloro-4-fluorophenyl)-11-methyl-6,7,10,11- | >4000 |
| | tetrahydro-5H-pyrido[4′,3′:3,4]pyrazolo[1,5-a][1,2,4]triazolo[3,4- | |
| | c][1,4]diazepine-12(13H)-carboxamide; | |
| 24 | N-(3-Cyano-4-fluorophenyl)-6,7,10,11-tetrahydro-5H- | 910 |
| | pyridazino[3,4-c]pyrido[4′,3′:3,4]pyrazolo[1,5-a]azepine-12(13H)- | |
| | carboxamide; | |
| 25 | N-(3-Chloro-4-fluorophenyl)-4,5,6,9,10,12- | 28 |
| | hexahydropyrazolo[3,4-c]pyrido[4′,3′:3,4]pyrazolo[1,5-a]azepine- | |
| | 11(2H)-carboxamide; | |
| 26 | N-(3-Cyano-4-fluorophenyl)-4,5,6,9,10,12- | 23 |
| | hexahydropyrazolo[3,4-c]pyrido[4′,3′:3,4]pyrazolo[1,5-a]azepine- | |
| | 11(2H)-carboxamide; | |
| 27 | N-(3-Cyano-4-fluorophenyl)-6,7,10,11-tetrahydro-5H-pyrido[2,3- | 170 |
| | c]pyrido[4′,3′:3,4]pyrazolo[1,5-a]azepine-12(13H)-carboxamide; | |
| 28 | N-(4Fluoro-3-(trifluoromethyl)phenyl)-6,7,10,11-tetrahydro-5H- | 1800 |
| | pyrido[2,3-c]pyrido[4′,3′:3,4] | |
| | pyrazolo[1,5-a]azepine-12(13H)-carboxamide; | |
| 29 | N-(3-Chloro-4-fluorophenyl)-2-methyl-4,5,6,9,10,12- | 3100 |
| | hexahydropyrazolo[3,4-c]pyrido[4′,3′:3,4]pyrazolo[1,5-a]azepine- | |
| | 11(2H)-carboxamide; | |
| 30 | N-(3-Chloro-4-fluorophenyl)-1-methyl-4,5,6,9,10,12- | 3400 |
| | hexahydropyrazolo[3,4-c]pyrido[4′,3′:3,4]pyrazolo[1,5-a]azepine- | |
| | 11(1H)-carboxamide; | |
| 31 | N-(3-Chloro-4-fluorophenyl)-5,6,9,10-tetrahydro-4H- | 9 |
| | isoxazolo[3,4-c]pyrido | |
| | [4′,3′:3,4]pyrazolo[1,5-a]azepine-11(12H)-carboxamide; | |
| 32 | N-(3Chloro-4-fluorophenyl)-5,6,9,10-tetrahydro-4H-isoxazolo[5,4- | 12 |
| | c]pyrido | |
| | [4′,3′:3,4]pyrazolo[1,5-a]azepine-11(12H)-carboxamide; | |
| 33 | N-(3-Cyano-4-fluorophenyl)-5,6,9,10-tetrahydro-4H- | 790 |
| | isoxazolo[5″,4″:3′,4′]cyclohepta[1′,2′:3,4]pyrazolo[1,5-a]pyrazine- | |
| | 11(12H)-carboxamide; | |
| 34 | N-(3-Cyano-4-fluorophenyl)-5,6,9,10-tetrahydro-4H- | 440 |
| | isoxazolo[5″,4″:3′,4′]cyclohepta[1′,2′:3,4]pyrazolo[1,5-a]pyrazine- | |
| | 11(12H)-carboxamide; | |
| 35 | N-(3-Cyano-4-fluorophenyl)-5,6,9,10-tetrahydro-4H- | 170 |
| | isoxazolo[3″,4″:3′,4′]cyclohepta[1′,2′:3,4]pyrazolo[1,5-a]pyrazine- | |
| | 11(12H)-carboxamide; and | |
| 36 | N-(4-Fluoro-3-(trifluoromethyl)phenyl)-5,6,9,10-tetrahydro-4H- | 180 |
| | isoxazolo[3″,4″:3′,4′]cyclohepta[1′,2′:3,4]pyrazolo[1,5-a]pyrazine- | |
| | 11(12H)-carboxamide; | |

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A compound of Formula (I) 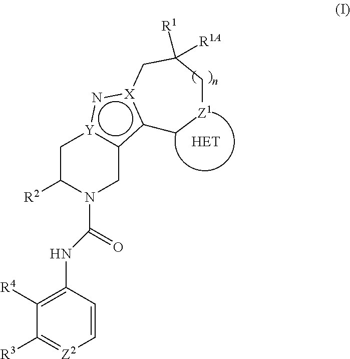
wherein
R¹ is independently selected from the group consisting of: hydrogen, C₁₋₄alkyl, hydroxy, hydroxymethyl, (2,2-difluoroethoxy)methyl, OC₁₋₄alkyl, and fluoro;
R^{1A} is independently hydrogen or taken together with R¹ to form methylenyl;
n is an integer that is 0, 1, or 2;
R² is independently selected from the group consisting of: hydrogen and C₁₋₆alkyl;
R³ is selected from the group consisting of: Cl, CN, and C₁₋₄haloalkyl;
R⁴ is H, or F;
HET is a 5- or 6-membered heteroaryl, optionally independently substituted with one to two substituents selected from the group consisting of: C₁₋₄alkyl, bromo, chloro, fluoro, and hydroxy(C₁₋₄)alkyl;
X and Y are each independently N or C, such that only one of X and Y is N in any instance;
Z¹ is N or C; and
Z² is N or CF.

2. The compound of claim 1, wherein R¹ is independently selected from the group consisting of: hydrogen, C₁₋₄alkyl, hydroxy, hydroxymethyl, (2,2-difluoroethoxy)methyl, OC₁₋₄alkyl, and fluoro.

3. The compound of claim 1, wherein R¹ and R^{1A} are taken together to form methylenyl.

4. The compound of claim 1, wherein n is 1.

5. (canceled)

6. (canceled)

7. The compound of claim 1, wherein R² is H or CH₃.

8. (canceled)

9. (canceled)

10. The compound of claim 1, wherein R³ is Cl, CN, or CF₃.

11. (canceled)

12. (canceled)

13. The compound of claim 1, wherein Y is N and X is C.

14. The compound of claim 1, wherein Y is C and X is N.

15. (canceled)

16. (canceled)

17. (canceled)

18. (canceled)

19. The compound of claim 1, wherein 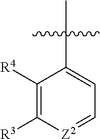
is 3-cyano-4-fluorophenyl, 4-fluoro-3-(trifluoromethyl)phenyl, or 3-chloro-4-fluorophenyl.

20. The compound of claim 1, wherein 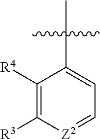
is 3-cyano-4-fluorophenyl.

21. The compound of claim 1 wherein HET is a heteroaryl independently selected from the group consisting of: isoxazolyl, pyridinyl, triazolyl, 3-methyl-triazolyl, pyridazinyl, pyrazolyl, and 1-methylpyrazolyl.

22. The compound of claim 1, wherein HET is a heteroaryl independently selected from the group consisting of isoxazolyl and pyrazolyl.

23. A compound selected from the group consisting of:
N-(3-Cyano-4-fluorophenyl)-5-methylene-5,6,9,10-tetrahydro-4H-isoxazolo[3,4-c]pyrido[4′,3′:3,4]pyrazolo[1,5-a]azepine-11(12H)-carboxamide;
N-(4-Fluoro-3-(trifluoromethyl)phenyl)-5-methylene-5,6,9,10-tetrahydro-4H-isoxazolo[3,4-c]pyrido[4′,3′:3,4]pyrazolo[1,5-a]azepine-11(12H)-carboxamide;
N-(3-Cyano-4-fluorophenyl)-5-(hydroxymethyl)-5,6,9,10-tetrahydro-4H-isoxazolo[3,4-c]pyrido[4′,3′:3,4]pyrazolo[1,5-a]azepine-11(12H)-carboxamide;
N-(4-Fluoro-3-(trifluoromethyl)phenyl)-5-(hydroxymethyl)-5,6,9,10-tetrahydro-4H-isoxazolo[3,4-c]pyrido[4′,3′:3,4]pyrazolo[1,5-a]azepine-11(12H)-carboxamide;
(5S*)—N-(3-Cyano-4-fluorophenyl)-5-((2,2-difluoroethoxy)methyl)-5,6,9,10-tetrahydro-4H-isoxazolo[3,4-c]pyrido[4′,3′:3,4]pyrazolo[1,5-a]azepine-11(12H)-carboxamide;
(5S*)-5-((2,2-Difluoroethoxy)methyl)-N-(4-fluoro-3-(trifluoromethyl)phenyl)-5,6,9,10-tetrahydro-4H-isoxazolo[3,4-c]pyrido[4′,3′:3,4]pyrazolo[1,5-a]azepine-11(12H)-carboxamide;
(5R*)—N-(3-Cyano-4-fluorophenyl)-5-((2,2-difluoroethoxy)methyl)-5,6,9,10-tetrahydro-4H-isoxazolo[3,4-c]pyrido[4′,3′:3,4]pyrazolo[1,5-a]azepine-11(12H)-carboxamide;
(5R*)-5-((2,2-Difluoroethoxy)methyl)-N-(4-fluoro-3-(trifluoromethyl)phenyl)-5,6,9,10-tetrahydro-4H-isoxazolo[3,4-c]pyrido[4′,3′:3,4]pyrazolo[1,5-a]azepine-11(12H)-carboxamide;
N-(3-Cyano-4-fluorophenyl)-5-methylene-5,6,9,10-tetrahydro-4H-isoxazolo[5,4-c]pyrido[4′,3′:3,4]pyrazolo[1,5-a]azepine-11(12H)-carboxamide;
N-(4-Fluoro-3-(trifluoromethyl)phenyl)-5-methylene-5,6,9,10-tetrahydro-4H-isoxazolo[5,4-c]pyrido[4′,3′:3,4]pyrazolo[1,5-a]azepine-11(12H)-carboxamide;
N-(3-Cyano-4-fluorophenyl)-5-hydroxy-5,6,9,10-tetrahydro-4H-isoxazolo[3,4-c]pyrido[4′,3′:3,4]pyrazolo[1,5-a]azepine-11(12H)-carboxamide;
N-(4-Fluoro-3-(trifluoromethyl)phenyl)-5-methyl-5,6,9,10-tetrahydro-4H-isoxazolo[3,4-c]pyrido[4′,3′:3,4]pyrazolo[1,5-a]azepine-11(12H)-carboxamide;
N-(3-Cyano-4-fluorophenyl)-5-methyl-5,6,9,10-tetrahydro-4H-isoxazolo[3,4-c]pyrido[4′,3′:3,4]pyrazolo[1,5-a]azepine-11(12H)-carboxamide;
(10R)—N-(3-Cyano-4-fluorophenyl)-10-methyl-5,6,9,10-tetrahydro-4H-isoxazolo[5,4-c]pyrido[4′,3′:3,4]pyrazolo[1,5-a]azepine-11(12H)-carboxamide;
(10R)—N-(4-Fluoro-3-(trifluoromethyl)phenyl)-10-methyl-5,6,9,10-tetrahydro-4H-isoxazolo[5,4-c]pyrido[4′,3′:3,4]pyrazolo[1,5-a]azepine-11(12H)-carboxamide;
(11R)—N-(3-Cyano-4-fluorophenyl)-11-methyl-6,7,10,11-tetrahydro-5H-pyrido[2,3-c]pyrido[4′,3′:3,4]pyrazolo[1,5-a]azepine-12(13H)-carboxamide;
(11R)—N-(4-Fluoro-3-(trifluoromethyl)phenyl)-11-methyl-6,7,10,11-tetrahydro-5H-pyrido[2,3-c]pyrido[4′,3′:3,4]pyrazolo[1,5-a]azepine-12(13H)-carboxamide;
(10R)—N-(3-Cyano-4-fluorophenyl)-10-methyl-5,6,9,10-tetrahydro-4H-isoxazolo[3,4-c]pyrido[4′,3′:3,4]pyrazolo[1,5-a]azepine-11(12H)-carboxamide;
(10R)—N-(4-Fluoro-3-(trifluoromethyl)phenyl)-10-methyl-5,6,9,10-tetrahydro-4H-isoxazolo[3,4-c]pyrido[4′,3′:3,4]pyrazolo[1,5-a]azepine-11(12H)-carboxamide;
N-(3-Chloro-4-fluorophenyl)-6,7,10,11-tetrahydro-5H-pyrido[4′,3′:3,4]pyrazolo[1,5-a][1,2,4]triazolo[3,4-c][1,4]diazepine-12(13H)-carboxamide;
N-(3-Chloro-4-fluorophenyl)-3-methyl-6,7,10,11-tetrahydro-5H-pyrido[4′,3′:3,4]pyrazolo[1,5-a][1,2,4]triazolo[3,4-c][1,4]diazepine-12(13H)-carboxamide;
(11R)—N-(3-Chloro-4-fluorophenyl)-11-methyl-6,7,10,11-tetrahydro-5H-pyrido[4′,3′:3,4]pyrazolo[1,5-a][1,2,4]triazolo[3,4-c][1,4]diazepine-12(13H)-carboxamide;
(11R)—N-(3-Chloro-4-fluorophenyl)-11-methyl-6,7,10,11-tetrahydro-5H-pyrido[4′,3′:3,4]pyrazolo[1,5-a][1,2,4]triazolo[3,4-c][1,4]diazepine-12(13H)-carboxamide;
N-(3-Cyano-4-fluorophenyl)-6,7,10,11-tetrahydro-5H-pyridazino[3,4-c]pyrido[4′,3′:3,4]-pyrazolo[1,5-a]azepine-12(13H)-carboxamide;
N-(3-Chloro-4-fluorophenyl)-4,5,6,9,10,12-hexahydropyrazolo[3,4-c]pyrido[4′,3′:3,4]-pyrazolo[1,5-a]azepine-11(2H)-carboxamide;
N-(3-Cyano-4-fluorophenyl)-4,5,6,9,10,12-hexahydropyrazolo[3,4-c]pyrido[4′,3′:3,4]-pyrazolo[1,5-a]azepine-11(2H)-carboxamide;
N-(3-Cyano-4-fluorophenyl)-6,7,10,11-tetrahydro-5H-pyrido[2,3-c]pyrido[4′,3′:3,4]-pyrazolo[1,5-a]azepine-12(13H)-carboxamide;
N-(4-Fluoro-3-(trifluoromethyl)phenyl)-6,7,10,11-tetrahydro-5H-pyrido[2,3-c]pyrido-[4′,3′:3,4]pyrazolo[1,5-a]azepine-12(13H)-carboxamide;
N-(3-Chloro-4-fluorophenyl)-2-methyl-4,5,6,9,10,12-hexahydropyrazolo[3,4-c]pyrido-[4′,3′:3,4]pyrazolo[1,5-a]azepine-11(2H)-carboxamide;
N-(3-Chloro-4-fluorophenyl)-1-methyl-4,5,6,9,10,12-hexahydropyrazolo[3,4-c]pyrido-[4′,3′:3,4]pyrazolo[1,5-a]azepine-11(1H)-carboxamide;
N-(3-Chloro-4-fluorophenyl)-5,6,9,10-tetrahydro-4H-isoxazolo[3,4-c]pyrido[4′,3′:3,4]-pyrazolo[1,5-a]azepine-11(12H)-carboxamide;
N-(3-Chloro-4-fluorophenyl)-5,6,9,10-tetrahydro-4H-isoxazolo[5,4-c]pyrido[4′,3′:3,4]-pyrazolo[1,5-a]azepine-11(12H)-carboxamide;
N-(3-Cyano-4-fluorophenyl)-5,6,9,10-tetrahydro-4H-isoxazolo[5″,4″:3′,4′]cyclohepta-[1′,2′:3,4]pyrazolo[1,5-a]pyrazine-11(12H)-carboxamide;
N-(3-Cyano-4-fluorophenyl)-5,6,9,10-tetrahydro-4H-isoxazolo[5″,4″:3′,4′]cyclohepta-[1′,2′:3,4]pyrazolo[1,5-a]pyrazine-11(12H)-carboxamide;
N-(3-Cyano-4-fluorophenyl)-5,6,9,10-tetrahydro-4H-isoxazolo[3″,4″:3′,4′]cyclohepta-[1′,2′:3,4]pyrazolo[1,5-a]pyrazine-11(12H)-carboxamide; and
N-(4-Fluoro-3-(trifluoromethyl)phenyl)-5,6,9,10-tetrahydro-4H-isoxazolo[3″,4″:3′,4′]-cyclohepta[1′,2′:3,4]pyrazolo[1,5-a]pyrazine-11(12H)-carboxamide;
or a pharmaceutically acceptable salt form thereof.

24. The compound of claim 23, wherein the compound is selected from the group consisting of
N-(3-Cyano-4-fluorophenyl)-5-methylene-5,6,9,10-tetrahydro-4H-isoxazolo[3,4-c]pyrido[4′,3′:3,4]pyrazolo[1,5-a]azepine-11(12H)-carboxamide;
N-(3-Cyano-4-fluorophenyl)-5-(hydroxymethyl)-5,6,9,10-tetrahydro-4H-isoxazolo[3,4-c]pyrido[4′,3′:3,4]pyrazolo[1,5-a]azepine-11(12H)-carboxamide;
N-(4-Fluoro-3-(trifluoromethyl)phenyl)-5-(hydroxymethyl)-5,6,9,10-tetrahydro-4H-isoxazolo[3,4-c]pyrido[4′,3′:3,4]pyrazolo[1,5-a]azepine-11(12H)-carboxamide;
(5S*)—N-(3-Cyano-4-fluorophenyl)-5-((2,2-difluoroethoxy)methyl)-5,6,9,10-tetrahydro-4H-isoxazolo[3,4-c]pyrido[4′,3′:3,4]pyrazolo[1,5-a]azepine-11(12H)-carboxamide;
(5R*)—N-(3-Cyano-4-fluorophenyl)-5-((2,2-difluoroethoxy)methyl)-5,6,9,10-tetrahydro-4H-isoxazolo[3,4-c]pyrido[4′,3′:3,4]pyrazolo[1,5-a]azepine-11(12H)-carboxamide;
(10R)—N-(3-Cyano-4-fluorophenyl)-10-methyl-5,6,9,10-tetrahydro-4H-isoxazolo[3,4-c]pyrido[4′,3′:3,4]pyrazolo[1,5-a]azepine-11(12H)-carboxamide;
N-(3-Chloro-4-fluorophenyl)-4,5,6,9,10,12-hexahydropyrazolo[3,4-c]pyrido[4′,3′:3,4]-pyrazolo[1,5-a]azepine-11(2H)-carboxamide;
N-(3-Cyano-4-fluorophenyl)-4,5,6,9,10,12-hexahydropyrazolo[3,4-c]pyrido[4′,3′:3,4]-pyrazolo[1,5-a]azepine-11(2H)-carboxamide;
N-(3-Chloro-4-fluorophenyl)-5,6,9,10-tetrahydro-4H-isoxazolo[3,4-c]pyrido[4′,3′:3,4]-pyrazolo[1,5-a]azepine-11(12H)-carboxamide; and
N-(3-Chloro-4-fluorophenyl)-5,6,9,10-tetrahydro-4H-isoxazolo[5,4-c]pyrido[4′,3′:3,4]-pyrazolo[1,5-a]azepine-11(12H)-carboxamide;
or a pharmaceutically acceptable salt form thereof.

25. A pharmaceutical composition comprising:
(A) at least one compound selected from compounds of Formula (I): 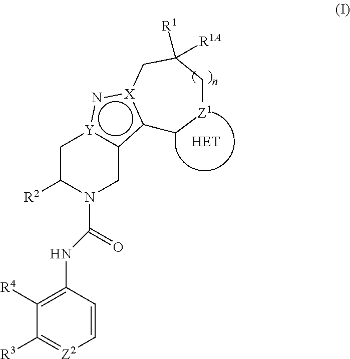
wherein:
R¹ is independently selected from the group consisting of: hydrogen, C₁₋₄alkyl, hydroxy, hydroxymethyl, (2,2-difluoroethoxy)methyl, OC₁₋₄alkyl, and fluoro;
R^{1A} is independently hydrogen or taken together with R¹ to form methylenyl;
n is an integer that is 0, 1, or 2;
R² is independently selected from the group consisting of: hydrogen and C₁₋₆alkyl;
R³ is selected from the group consisting of: Cl, CN, and C₁₋₄haloalkyl;
R⁴ is H, or F;
HET is a 5- or 6-membered heteroaryl, optionally independently substituted with one to two substituents selected from the group consisting of: C₁₋₄alkyl, bromo, chloro, fluoro, and hydroxy(C₁₋₄)alkyl;
X and Y are each independently N or C, such that only one of X and Y is N in any instance;
Z¹ is N or C; and
Z² is N or CF;
and pharmaceutically acceptable salts, solvates, stereoisomers, isotopic variants, or N-oxides of compounds of Formula (I); and
(B) at least one pharmaceutically acceptable excipient.

26. A pharmaceutical composition comprising at least one compound of claim 23 and at least one pharmaceutically acceptable excipient.

27. A method of treating an HBV infection in an individual in need thereof, comprising administering to the individual a therapeutically effective amount of at least one compound of claim 1.

28. A method of inhibiting or reducing the formation or presence of HBV DNA-containing particles or HBV RNA-containing particles in an individual in need thereof, comprising administering to the individual a therapeutically effective amount of a compound of claim 1.

29. The method of claim 27, further comprising administering to the individual at least one additional therapeutic agent.

30. (canceled)

31. (canceled)

32. (canceled)

33. (canceled)

34. The method of claim 28, further comprising administering to the individual at least one additional therapeutic agent.
